(19) United States
(12) Patent Application Publication
(10) Pub. No.: US 2025/0032645 A1
(43) Pub. Date: Jan. 30, 2025

(54) COMPOSITIONS AND METHODS FOR THE TREATMENT OF NONALCOHOLIC STEATOHEPATITIS (NASH) FIBROSIS

(71) Applicant: The Trustees of Columbia University in the City of New York, New York, NY (US)

(72) Inventors: Ira TABAS, New City, NY (US); Xiaobo WANG, Fort Lee, NJ (US); Mary MOORE, New York, NY (US)

(21) Appl. No.: 18/795,070

(22) Filed: Aug. 5, 2024

Related U.S. Application Data
(63) Continuation-in-part of application No. PCT/US23/61933, filed on Feb. 3, 2023.
(60) Provisional application No. 63/306,950, filed on Feb. 4, 2022.

(51) Int. Cl. A61K 48/00; A61K 38/45; C12N 15/86

(52) U.S. Cl. CPC A61K 48/0058; A61K 38/45; C12N 15/86; C12N 2750/14143

(57) ABSTRACT

Compositions and methods for treating or preventing nonalcoholic steatohepatitis (NASH) fibrosis, also known as MASH. In one aspect, the disclosed methods relate to targeting a MBOAT7 risk variant, rs641738, in order to increase MBOAT7 expression and down-stream signaling or to silence TAZ. The compositions and methods disclosed herein can be used as disease modifying therapies to enable treatment of NASH fibrosis/MASH and related disorders earlier in disease progression and improve clinical outcomes.

### CROSS-REFERENCE TO RELATED APPLICATION

This application is a Continuation-in-Part of International Application No. PCT/US23/61933 filed on Feb. 3, 2023, which claims the benefit of U.S. Provisional Application No. 63/306,950, filed Feb. 4, 2022, entitled “COMPOSITIONS AND METHODS FOR THE TREATMENT OF NONALCOHOLIC STEATOHEPATITIS (NASH) FIBROSIS”, the contents of which are hereby incorporated by reference.

### STATEMENT REGARDING FEDERALLY SPONSORED RESEARCH OR DEVELOPMENT

This invention was made with government support under DK116620 awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Oct. 8, 2024, is named 0019240_01258US2_SL.xml and is 90,901 bytes in size.

### BACKGROUND

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosure of these publications in their entireties are hereby incorporated by reference into this application in order to more fully describe the state of the art as known to those skilled therein as of the date of the invention described herein.

Nonalcoholic steatohepatitis (NASH) or metabolic dysfunction-associated steatohepatitis (MASH) is one of the leading causes of chronic liver disease. Genome-wide association studies (GWAS) have identified a common liver disease susceptibility locus, rs641738, to be associated with an increased risk of nonalcoholic fatty liver disease, and its more advanced form, NASH or MASH. This risk variant is associated with reduced expression and activity of membrane bound O-acyltransferase domain containing 7 (MBOAT7), which has also been found to be decreased in NASH or MASH progression in the general human NASH or MASH population and mouse models of diet-induced NASH or MASH. There is a need for human genetic-based therapy for NASH or MASH. The need to for this type of therapy is great, as there are currently no approved drugs to treat NASH or halt its progression into liver fibrosis or cirrhosis. Thus, there exists a need for compositions and methods for treating NASH fibrosis or MASH, and basing this on human genetics would add tremendous value to this effort.

### SUMMARY

In one aspect, the present application relates to methods of treating or preventing nonalcoholic steatohepatitis (NASH) in a subject in need thereof, comprising administering to said patient a composition that targets the hepatic TAZ pathway. In various embodiments, the composition inhibits TAZ expression. In various embodiments, the composition comprises a TAZ siRNA. In various embodiments, the composition increases MBOAT7 expression. In various embodiments the composition comprises either SEQ ID NO: 1 or 2. In various embodiments, the composition comprises an mRNA nanoparticle. In various embodiments, the composition is a viral vector. In various embodiments, the viral vector is an adeno-associated vector (AAV). In various embodiments the viral vector is AAV8. In various embodiments the patient is a mammal. In various embodiments, the mammal is a human. In various embodiments the human expresses the rs641738 variant of the MBOAT7 gene.

In various embodiments, the invention disclosed herein relates to a method of treating or preventing NASH in a subject in need thereof comprising (i) identifying the patient as expressing the rs641738 variant and (ii) administering to said patient a composition that targets the hepatic TAZ pathway. In various embodiments, the composition inhibits TAZ expression. In various embodiments, the composition comprises a TAZ siRNA. In various embodiments, the composition increases MBOAT7 expression. In various embodiments, the composition comprises MBOAT7 mRNA. In various embodiments, the composition comprises either SEQ ID NO: 1 or 2. In various embodiments, the composition comprises an mRNA encoding either SEQ ID NO: 3 or 4. In various embodiments, the composition comprises an mRNA nanoparticle. In various embodiments, the composition is a viral vector. In various embodiments, the viral vector is an AAV. In various embodiments, the viral vector is AAV8. In various embodiments, the patient is a mammal. In various embodiments the mammal is a human.

In various embodiments, the invention disclosed herein relates to a composition for treating or preventing NASH, comprising an expression vector capable of targeting the hepatic TAZ pathway. In various embodiments, the composition inhibits TAZ expression. In various embodiments, the composition comprises a TAZ siRNA. In various embodiments the composition increases MBOAT7 expression. In various embodiments, the composition comprises MBOAT7 mRNA. In various embodiments the composition comprises either SEQ ID NO: 1 or 2. In various embodiments, the composition comprises an mRNA encoding either SEQ ID NO: 3 or 4. In various embodiments the composition comprises an mRNA nanoparticle. In various embodiments the composition is a viral vector. In various embodiments, the viral vector is an AAV vector. In various embodiments the viral vector is AAV8.

In certain aspects, the present application relates to a method of treating or preventing metabolic dysfunction-associated steatohepatitis (MASH) in a subject in need thereof, the method comprising administering to the subject a composition targeting the hepatic TAZ pathway. In some embodiments, the composition reduces or inhibits TAZ expression in the subject when compared to untreated subjects or to expression level of TAZ in the subject pre-treatment. In some embodiments, the composition reduces or inhibits Indian hedgehog (IHH) expression in the subject when compared to untreated subjects or to expression level of IHH in the subject pre-treatment. In some embodiments, the composition reduces or inhibits PS synthase-1 (PSS1) expression in the subject when compared to untreated subjects or to expression level of PSS1 in the subject pre-treatment. In some embodiments, the composition reduces or inhibits Aster-B/C expression in the subject when compared to untreated subjects or to expression level of Aster-B/C in the subject pre-treatment. In some embodiments, the composition comprises an Aster-B/C small interfering ribonucleic acid (siRNA). In some embodiments, the composition comprises either SEQ ID NO: 15 or 16. In some embodiments, the composition reduces or inhibits 1-acylglycerol-3-phosphate O-acyltransferase (AGPAT1) in the subject when compared to untreated subjects or to expression level of AGPAT1 in the subject pre-treatment. In some embodiments, the composition reduces or inhibits CDP-diacylglycerol (DAG) synthase-2 (CDS2) in the subject when compared to untreated subjects or to expression level of CDS2 in the subject pre-treatment. In some embodiments, the composition comprises a CDS2 small interfering ribonucleic acid (siRNA). In some embodiments, the composition comprises SEQ ID NO: 17. In some embodiments, the composition reduces or inhibits diacylglycerol (DAG) in the subject when compared to untreated subjects or to expression level of DAG in the subject pre-treatment. In some embodiments, the composition reduces or inhibits phosphatidylinositol (PI) in the subject when compared to untreated subjects or to expression level of PI in the subject pre-treatment. In some embodiments, the composition increases MBOAT7 expression. In some embodiments, the composition comprises MBOAT7 mRNA. In some embodiments, the composition comprises either SEQ ID NO: 1 or 2. In some embodiments, the composition comprises an mRNA encoding either SEQ ID NO: 3 or 4. In some embodiments, the composition comprises an mRNA nanoparticle. In various embodiments the composition comprises a viral vector comprising a nucleic acid encoding MBOAT7. In various embodiments, the viral vector is an AAV. In various embodiments the viral vector is AAV8. In some embodiments, the patient is a mammal. In some embodiments, the mammal is a human. In some embodiments, the human expresses the rs641738 variant of the MBOAT7 gene. In some embodiments, the composition comprises a guide RNA (gRNA) or a single-molecule guide RNA (sgRNA) comprising a spacer sequence that is complementary to a portion of a nucleic acid sequence encoding TAZ, IHH, PSS1, Aster-B/C, AGPAT1, CD2, DAG or PI. In some embodiments, the gRNA or the sgRNA comprises SEQ ID NO: 11 or 12. In some embodiments, the gRNA or the sgRNA is pre-complexed with a DNA endonuclease. In some embodiments, the DNA endonuclease is a Cas9 endonuclease.

In certain aspects, the present application relates to a method of treating or preventing metabolic dysfunction-associated steatohepatitis (MASH) in a subject in need thereof, the method comprising administering to the subject a composition that inhibits a cholesterol trafficking pathway. In some embodiments, the composition reduces or inhibits TAZ expression in the subject when compared to untreated subjects or to expression level of TAZ in the subject pre-treatment. In some embodiments, the composition reduces or inhibits Indian hedgehog (IHH) expression in the subject when compared to untreated subjects or to expression level of IHH in the subject pre-treatment. In some embodiments, the composition reduces or inhibits PS synthase-1 (PSS1) expression in the subject when compared to untreated subjects or to expression level of PSS1 in the subject pre-treatment. In some embodiments, the composition reduces or inhibits Aster-B/C expression in the subject when compared to untreated subjects or to expression level of Aster-B/C in the subject pre-treatment. In some embodiments, the composition comprises an Aster-B/C small interfering ribonucleic acid (siRNA). In some embodiments, the composition comprises either SEQ ID NO: 15 or 16. In some embodiments, the composition reduces or inhibits 1-acylglycerol-3-phosphate O-acyltransferase (AGPAT1) in the subject when compared to untreated subjects or to expression level of AGPAT1 in the subject pre-treatment. In some embodiments, the composition reduces or inhibits CDP-diacylglycerol (DAG) synthase-2 (CDS2) in the subject when compared to untreated subjects or to expression level of CDS2 in the subject pre-treatment. In some embodiments, the composition comprises a CDS2 small interfering ribonucleic acid (siRNA). In some embodiments, the composition comprises SEQ ID NO: 17. In some embodiments, the composition reduces or inhibits diacylglycerol (DAG) in the subject when compared to untreated subjects or to expression level of DAG in the subject pre-treatment. In some embodiments, the composition reduces or inhibits phosphatidylinositol (PI) in the subject when compared to untreated subjects or to expression level of PI in the subject pre-treatment. In some embodiments, the composition increases MBOAT7 expression. In some embodiments, the composition comprises MBOAT7 mRNA. In some embodiments, the composition comprises either SEQ ID NO: 1 or 2. In some embodiments, the composition comprises an mRNA encoding either SEQ ID NO: 3 or 4. In some embodiments, the composition comprises an mRNA nanoparticle. In various embodiments the composition comprises a viral vector comprising a nucleic acid encoding MBOAT7. In various embodiments, the viral vector is an AAV. In various embodiments the viral vector is AAV8. In some embodiments, the patient is a mammal. In some embodiments, the mammal is a human. In some embodiments, the human expresses the rs641738 variant of the MBOAT7 gene. In some embodiments, the composition comprises a guide RNA (gRNA) or a single-molecule guide RNA (sgRNA) comprising a spacer sequence that is complementary to a portion of a nucleic acid sequence encoding TAZ, IHH, PSS1, Aster-B/C, AGPAT1, CD2, DAG or PI. In some embodiments, the gRNA or the sgRNA comprises SEQ ID NO: 11 or 12. In some embodiments, the gRNA or the sgRNA is pre-complexed with a DNA endonuclease. In some embodiments, the DNA endonuclease is a Cas9 endonuclease.

In certain aspects, the present application relates to a method of treating or preventing metabolic dysfunction-associated steatohepatitis (MASH) in a subject in need thereof, the method comprising administering to the subject a composition that inhibits a PL metabolism pathway. In some embodiments, the composition reduces or inhibits TAZ expression in the subject when compared to untreated subjects or to expression level of TAZ in the subject pre-treatment. In some embodiments, the composition reduces or inhibits Indian hedgehog (IHH) expression in the subject when compared to untreated subjects or to expression level of IHH in the subject pre-treatment. In some embodiments, the composition reduces or inhibits PS synthase-1 (PSS1) expression in the subject when compared to untreated subjects or to expression level of PSS1 in the subject pre-treatment. In some embodiments, the composition reduces or inhibits Aster-B/C expression in the subject when compared to untreated subjects or to expression level of Aster-B/C in the subject pre-treatment. In some embodiments, the composition comprises an Aster-B/C small interfering ribonucleic acid (siRNA). In some embodiments, the composition comprises either SEQ ID NO: 15 or 16. In some embodiments, the composition reduces or inhibits 1-acylglycerol-3-phosphate O-acyltransferase (AGPAT1) in the subject when compared to untreated subjects or to expression level of AGPAT1 in the subject pre-treatment. In some embodiments, the composition reduces or inhibits CDP-diacylglycerol (DAG) synthase-2 (CDS2) in the subject when compared to untreated subjects or to expression level of CDS2 in the subject pre-treatment. In some embodiments, the composition comprises a CDS2 small interfering ribonucleic acid (siRNA). In some embodiments, the composition comprises SEQ ID NO: 17. In some embodiments, the composition reduces or inhibits diacylglycerol (DAG) in the subject when compared to untreated subjects or to expression level of DAG in the subject pre-treatment. In some embodiments, the composition reduces or inhibits phosphatidylinositol (PI) in the subject when compared to untreated subjects or to expression level of PI in the subject pre-treatment. In some embodiments, the composition increases MBOAT7 expression. In some embodiments, the composition comprises MBOAT7 mRNA. In some embodiments, the composition comprises either SEQ ID NO: 1 or 2. In some embodiments, the composition comprises an mRNA encoding either SEQ ID NO: 3 or 4. In some embodiments, the composition comprises an mRNA nanoparticle. In some embodiments, the composition comprises a viral vector comprising a nucleic acid encoding MBOAT7. In various embodiments, the viral vector is an AAV. In various embodiments the viral vector is AAV8. In some embodiments, the patient is a mammal. In some embodiments, the mammal is a human. In some embodiments, the human expresses the rs641738 variant of the MBOAT7 gene. In some embodiments, the composition comprises a guide RNA (gRNA) or a single-molecule guide RNA (sgRNA) comprising a spacer sequence that is complementary to a portion of a nucleic acid sequence encoding TAZ, IHH, PSS1, Aster-B/C, AGPAT1, CD2, DAG or PI. In some embodiments, the gRNA or the sgRNA comprises SEQ ID NO: 11 or 12. In some embodiments, the gRNA or the sgRNA is pre-complexed with a DNA endonuclease. In some embodiments, the DNA endonuclease is a Cas9 endonuclease.

In certain aspects, the present application relates to a composition for treating or preventing metabolic dysfunction-associated steatohepatitis (MASH), comprising an expression vector capable of targeting the hepatic TAZ pathway. In some embodiments, the composition reduces or inhibits expression of one or more of TAZ, IHH, PSS1, Aster-B/C, AGAPT1, CDS2, DAG2 or PI. In some embodiments, the composition comprises an Aster-B/C small interfering ribonucleic acid (siRNA). In some embodiments, the composition comprises either SEQ ID NO: 15 or 16. In some embodiments, the composition comprises a CDS2 small interfering ribonucleic acid (siRNA). In some embodiments, the composition comprises SEQ ID NO: 17. In some embodiments, the composition comprises a viral vector comprising a nucleic acid encoding MBOAT7. In some embodiments, the composition comprises a viral vector. In some embodiments, the viral vector is an AAV vector. In some embodiments, the viral vector is AAV8. In some embodiments, the composition increases MBOAT7 expression. In some embodiments, the composition increases MBOAT7 expression. In some embodiments, the composition comprises MBOAT7 mRNA. In some embodiments, the composition comprises either SEQ ID NO: 1 or 2. In some embodiments, the composition comprises an mRNA encoding either SEQ ID NO: 3 or 4. In some embodiments, the composition comprises an mRNA nanoparticle. In some embodiments, the composition comprises a guide RNA (gRNA) or a single-molecule guide RNA (sgRNA) comprising a spacer sequence that is complementary to a portion of a nucleic acid sequence encoding TAZ, IHH, PSS1, Aster-B/C. AGPAT1, CD2, DAG or PI. In some embodiments, the gRNA or the sgRNA comprises SEQ ID NO: 11 or 12. In some embodiments, the gRNA or the sgRNA is pre-complexed with a DNA endonuclease. In some embodiments, the DNA endonuclease is a Cas9 endonuclease.

In certain aspects, the present application relates to a composition for treating or preventing metabolic dysfunction-associated steatohepatitis (MASH), comprising an expression vector capable of targeting a cholesterol trafficking pathway. In some embodiments, the composition reduces or inhibits expression of one or more of TAZ, IHH, PSS1, Aster-B/C, AGAPT1, CDS2, DAG2 or PI. In some embodiments, the composition comprises an Aster-B/C small interfering ribonucleic acid (siRNA). In some embodiments, the composition comprises either SEQ ID NO: 15 or 16. In some embodiments, the composition comprises a CDS2 small interfering ribonucleic acid (siRNA). In some embodiments, the composition comprises SEQ ID NO: 17. In some embodiments, the composition comprises a viral vector comprising a nucleic acid encoding MBOAT7. In some embodiments, the composition comprises a viral vector. In some embodiments, the viral vector is an AAV vector. In some embodiments, the viral vector is AAV8. In some embodiments, the composition increases MBOAT7 expression. In some embodiments, the composition increases MBOAT7 expression. In some embodiments, the composition comprises MBOAT7 mRNA. In some embodiments, the composition comprises either SEQ ID NO: 1 or 2. In some embodiments, the composition comprises an mRNA encoding either SEQ ID NO: 3 or 4. In some embodiments, the composition comprises an mRNA nanoparticle. In some embodiments, the composition comprises a guide RNA (gRNA) or a single-molecule guide RNA (sgRNA) comprising a spacer sequence that is complementary to a portion of a nucleic acid sequence encoding TAZ, IHH, PSS1, Aster-B/C. AGPAT1, CD2, DAG or PI. In some embodiments, the gRNA or the sgRNA comprises SEQ ID NO: 11 or 12. In some embodiments, the gRNA or the sgRNA is pre-complexed with a DNA endonuclease. In some embodiments, the DNA endonuclease is a Cas9 endonuclease.

In certain aspects, the present application relates to a composition for treating or preventing metabolic dysfunction-associated steatohepatitis (MASH), comprising an expression vector capable of targeting a PL metabolism pathway. In some embodiments, the composition reduces or inhibits expression of one or more of TAZ, IHH, PSS1, Aster-B/C, AGAPT1, CDS2, DAG2 or PI. In some embodiments, the composition comprises an Aster-B/C small interfering ribonucleic acid (siRNA). In some embodiments, the composition comprises either SEQ ID NO: 15 or 16. In some embodiments, the composition comprises a CDS2 small interfering ribonucleic acid (siRNA). In some embodiments, the composition comprises SEQ ID NO: 17. In some embodiments, the composition comprises a viral vector comprising a nucleic acid encoding MBOAT7. In some embodiments, the composition comprises a viral vector. In some embodiments, the viral vector is an AAV vector. In some embodiments, the viral vector is AAV8. In some embodiments, the composition increases MBOAT7 expression. In some embodiments, the composition increases MBOAT7 expression. In some embodiments, the composition comprises MBOAT7 mRNA. In some embodiments, the composition comprises either SEQ ID NO: 1 or 2. In some embodiments, the composition comprises an mRNA encoding either SEQ ID NO: 3 or 4. In some embodiments, the composition comprises an mRNA nanoparticle. In some embodiments, the composition comprises a guide RNA (gRNA) or a single-molecule guide RNA (sgRNA) comprising a spacer sequence that is complementary to a portion of a nucleic acid sequence encoding TAZ, IHH, PSS1, Aster-B/C. AGPAT1, CD2, DAG or PI. In some embodiments, the gRNA or the sgRNA comprises SEQ ID NO: 11 or 12. In some embodiments, the gRNA or the sgRNA is pre-complexed with a DNA endonuclease. In some embodiments, the DNA endonuclease is a Cas9 endonuclease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows livers that were assayed for MBOAT7 expression by IFM and quantification.

FIG. 1B shows immunohistochemistry H & E stains of liver sections and a bar chart indicating lipid droplet % area.

FIG. 1C shows immunohistochemistry F4/80 stains of liver sections and a bar chart indicating F4/80% area.

FIG. 1D shows histochemical Sirius red stains of liver sections and a bar chart indicating Sirius red % area.

FIG. 1E shows immunohistochemistry α-smooth muscle actin (αSMA) stains of liver sections and a bar chart indicating αSMA % area.

FIG. 1F shows immunohistochemistry COL1a1 positive area stains of liver sections and a bar chart indicating COL1a1 positive % area.

FIG. 1G shows immunohistochemistry OPN stains of liver sections and a bar chart indicating OPN % area.

FIG. 1H is a bar chart showing mRNA markers of hepatic stellate cell activation for AAV8-TBG-MBOAT7-treated mice compared with AAV8-TBG-GFP controls.

FIG. 2A shows an immunoblot of MBOAT7 and TAZ proteins in human primary hepatocytes.

FIG. 2B shows an immunoblot of MBOAT7 and TAZ proteins in mouse primary hepatocytes and a bar chart of TAZ:β-actin ratio.

FIG. 3A shows an immunoblot and bar charts of MBOAT7 and TAZ proteins in AML12 cells transfected with Scr or Mboat7 siRNA and control or Aster B/C ASO.

FIG. 3B provides bar charts of cholesterol ester and phosphatidylserine content of AMl12 cells treated with siMboatt7 or control siRNA.

FIG. 3C shows an immunoblot and bar charts of phospho-Creb^{ser133}:total CREB ratio from livers of AAV8-TBG-MBOAT7- or AAV8-TBG-GFP-treated mice.

FIG. 3D provides a bar chart of RhoA activity in siMboat7-vs. Control-treated AML12 hepatocytes.

FIG. 4A shows an immunoblot and bar charts of TAZ, IHH and MBOAT7 proteins from AML12 cells transfected with GFP control or MBOAT7 plasmid.

FIG. 4B shows an immunoblot and bar charts of TAZ protein and lower IHH protein from livers of AAV8-TBG-MBOAT7-treated mice from FIG. 1.

FIG. 5A is a bar chart showing body weight for AAV8-TBG-MBOAT7-treated mice compared with AAV8-TBG-GFP controls.

FIG. 5B is a bar chart showing liver/body weight for AAV8-TBG-MBOAT7-treated mice compared with AAV8-TBG-GFP controls.

FIG. 5C is a bar chart showing inguinal fat weight for AAV8-TBG-MBOAT7-treated mice compared with AAV8-TBG-GFP controls.

FIG. 5D is a bar chart showing plasma ALT for AAV8-TBG-MBOAT7-treated mice compared with AAV8-TBG-GFP controls.

FIG. 5E is a bar chart showing plasma AST for AAV8-TBG-MBOAT7-treated mice compared with AAV8-TBG-GFP controls.

FIG. 6A shows bar charts of total cholesterol ester and a spectrum of different cholesterol esters following AAV8-TBG-MBOAT7 treatment compared to GFP control.

FIG. 6B shows a bar chart of phosphatidylinositol (PI) following AAV8-TBG-MBOAT7 treatment compared to GFP control.

FIG. 6C shows a bar chart of PI:Total PI ratio following AAV8-TBG-MBOAT7 treatment compared to GFP control.

FIG. 7 shows a map of the MBOAT7 expressing AAV8-TBG-MBOAT7 vector.

FIG. 8 shows the graphical abstract, which illustrates that low MBOAT7 expression, a genetic risk for MASH, promotes liver fibrosis by activating a phospholipid-TAZ-IHH pathway in hepatocytes.

FIGS. 9A-H show hepatocyte MBOAT7 restoration after the development of hepatosteatosis reduces liver fibrosis and HSC activation in MASH. mice. Mice were fed the FPC for 8 weeks to induce hepatosteatosis and then injected with AAV8-TBG-Mboat7 or AAV8-TBG-GFP control (Ctrl) virus. The mice were continued on the FPC diet for an additional 8 weeks. Livers were assayed for (A) MBOAT7 by IFM (scale bar=50 μm); (B) Picrosirius red (scale bar=200 μm); (C) αSMA⁺ by IFM (scale bar=50 μm); (D, E) COL1A1 and OPN by IHC (scale bar=100 μm); (F) mRNAs associated with HSC activation, expressed relative to the MASH control value; (G) lipid-droplet area (scale bar=100 μm); and (H) F4/80⁺ area (scale bar=100 μm). The arrows in B-E show examples of positively stained cells. The values for all graphs are means±SEM. In (A), the data are expressed as MBOAT7⁺ area in hepatocytes, normalized to the chow-control value, and in B-E, G, and H, the data are expressed as percent area. n=6-7 mice/group and values are means±SEM. *p<0.05, **p<0.01, ***p<0.001, ****p<0.0001. Abbreviations: αSMA, alpha-smooth muscle actin; COL1A1, collagen 1a1; FPC, fructose-palmitate-cholesterol diet; IFM, immunofluorescence microscopy; IHC, xxx; MASH, metabolic dysfunction-associated steatohepatitis; MBOAT7, membrane-bound O-acyltransferase domain containing 7; OPN, osteopontin.

FIGS. 10A-H show hepatocyte MBOAT7 silencing after the development of hepatosteatosis accelerates liver fibrosis and HSC activation in MASH mice. Mice were fed the FPC for 8 weeks to induce hepatosteatosis and then injected with AAV8-H1-shMboat7 or AAV8-H1-shControl (shCtrl) virus. The mice were continued on the FPC diet for an additional 8 weeks. Livers were assayed for (A) MBOAT7 by IFM (scale bar=50 μm); (B) Picrosirius red (scale bar=200 μm); (C) αSMA⁺ by IFM (scale bar=50 μm); (D, E) COL1A1 and OPN by IHC (scale bar=100 μm); (F) mRNAs associated with HSC activation, expressed relative to the MASH control value; (G) lipid-droplet area (scale bar=100 μm); and (H) F4/80⁺ area (scale bar=100 μm). The arrows in B-E show examples of positively stained cells. The values for all graphs are means±SEM. In (A), the data are expressed as MBOAT7⁺ area per hepatocyte area, normalized to the MASH-shCtrl value, and in B-E, G, and H, the data are expressed as percent area. n=6-7 mice/group and values are means±SEM. *p<0.05, **p<0.01, ***p<0.001****, p<0.0001. Abbreviations: αSMA, alpha-smooth muscle actin; COL1A1, collagen 1a1; FPC, fructose-palmitate-cholesterol diet; IFM, immunofluorescence microscopy; IHC, xxx; MASH, metabolic dysfunction-associated steatohepatitis; MBOAT7, membrane-bound O-acyltransferase domain containing 7; OPN, osteopontin.

FIGS. 11A-G show evidence in hepatic cells linking MBOAT7 LoF to elevated TAZ/IHH and HSC activation. (A, B) Immunoblots of MBOAT7, TAZ, and IHH in human and mouse primary hepatocytes treated with siMboat7 or siCtrl, with densitometric quantification relative to β-actin (n=3 technical replicates/group). (C) Immunoblots of MBOAT7, TAZ, and IHH in AML12 hepatocytes transfected with plasmids encoding MBOAT7 or GFP control (Ctrl), with densitometric quantification (n=3 technical replicates/group). (D) Immunoblots of MBOAT7, TAZ, and IHH in wild-type versus Mboat7^{-/-} mouse primary hepatocytes treated for 6 hours with 0.4 mM oleic acid, with densitometric quantification (n=4 technical replicates/group). (E) Primary HSCs from wild-type mice were incubated for 48 hours with CM obtained from oleic acid-treated wild-type or Mboat7^{-/-} mouse primary hepatocytes or with medium not exposed to hepatocytes (Basal), with or without a Smothened (Smo) inhibitor to block hedgehog signaling in the HSCs. The HSCs were then assayed for Opn and Timp1 mRNA (n=4 technical replicates/group). (F, G) Human primary hepatic 3D spheroids were treated with siMBOAT7 or siCtrl and assayed for the indicated mRNAs. Values are means±SEM. *p<0.05, **p<0.01, ***p<0.001. Abbreviations: CM, conditioned medium; IHH, Indian hedgehog; MBOAT7, membrane-bound O-acyltransferase domain containing 7.

FIGS. 12A-D show evidence in experimental MASH and human liver linking MBOAT7 LoF to elevated TAZ. (A) Representative immunoblots of TAZ, IHH, and p-S89-TAZ in the livers of mice given AAV8-TBG-GFP (Ctrl) or AAV8-TBG-Mboat7 from FIG. 9, with densitometric quantification (n=6 mice/group). (B) Representative immunoblots of MBOAT7, TAZ, and IHH in the livers of mice given AAV8-H1-shCtrl or AAV8-H1-shMboat7 from FIG. 10, with densitometric quantification (n=6 mice/group). (C) The percent nuclear TAZ was assayed by IFM in liver sections from subjects with MASH and the indicated genotypes affecting MBOAT7 (n=30 CC, 50 TC, 25 TT), PNPLA3 (n=33 CC, 50 CG, 30 GG), and HSD17B13 (n=63 TT, 36 T/TA, 6 TA/TA). A one-way ANCOVA was performed with sex, age, BMI, and fasting blood glucose as covariates for MBOAT7 rs641738, PNPLA3 rs738409, and HSD17B13 rs72613567. (D) Liver specimens from obese individuals with the indicated genotypes affecting MBOAT7 (n=46 CC, 47 TC, 29 TT), PNPLA3 (n=60 CC, 56 CG, 9 GG), and HSD17B13 (n=82 TT, 40 T/TA, 3 TA/TA) were assayed for IHH mRNA, expressed relative to the first group for each locus. The data were log-transformed, and a one-way ANCOVA was performed with sex, age, fasting blood glucose, LDL-cholesterol, and HDL-cholesterol as covariates for MBOAT7 rs641738 and PNPLA3 rs738409, and sex, age, and body mass index as covariates for HSD17B13 rs72613567. Values are means±SEM. *p<0.05, **p<0.01, ****p<0.0001. Abbreviations: IFM, immunofluorescence microscopy; IHH, Indian hedgehog; LoF, loss-of-function; MASH, metabolic dysfunction-associated steatohepatitis; MBOAT7, membrane-bound O-acyltransferase domain containing 7.

FIGS. 13A-H show MBOAT7 LoF in hepatocytes enhances the cholesterol trafficking-activated TAZ pathway. (A) Immunoblots of MBOAT7 and TAZ in AML12 cells transfected with ASTER-B/C ASO or control and siMboat7 or control, with densitometric quantification relative to β-actin loading control (n=3 technical replicates/group). (B) Left, AML12 cells transfected with siMboat7 or control siRNA were incubated for 30 minutes±MβCD-cholesterol, incubated with His6-tagged ALOD4 (“His6” disclosed as SEQ ID NO: 70), fixed, incubated with anti-His6 (“His6” disclosed as SEQ ID NO: 70) and then Alexa Fluor 488-labeled secondary antibody (green), and counterstained with DAPI/nuclear (blue); arrows show increased His6-ALOD4 (“His6” disclosed as SEQ ID NO: 70) binding at the plasma membrane. Right, AML12 cells transfected with siMboat7 or control siRNA were incubated for 60 minutes±MβCD-cholesterol, incubated with His6-ALOD4 (“His6” disclosed as SEQ ID NO: 70), and then immunoblotted using anti-His6 (“His6” disclosed as SEQ ID NO: 70), with densitometric quantification relative to calnexin loading control (n=3 technical replicates/group). (C) AML12 cells were treated with siCtrl or siMboat7 and then incubated for 4 hours±MβCD-cholesterol. Cell extracts were assayed for markers of cholesterol biosynthesis through qPCR (n=3 technical replicates/group). (D) RhoA activity in control and Mboat7-silenced AML12 cells (n=6 technical replicates/group). (E) Phosphatidylserine content in control and Mboat7-silenced AML12 cells (n=3 technical replicates/group). (F) Left, Immunoblot of TAZ in wild-type and Ptdss1-knockdown (KD) AML12 cells. Right, Immunoblots of TAZ and MBOAT7 in Ptdss1-KD AML12 cells treated with control or siMboat7, with densitometric quantification (n=3 technical replicates/group). (G) Left, Immunoblot of TAZ in wild-type and Agpat1-knockdown (KD) AML12 cells. Right, Immunoblots of TAZ and MBOAT7 in Agpat1-KD AML12 cells treated with control or siMboat7, with densitometric quantification (n=3 technical replicates/group). (H) Immunoblots of MBOAT7 and TAZ in AML2 cells in control and Mboat7-silenced AML12 cells that were also treated with control or siCds2, with densitometric quantification (n=3 technical replicates/group). Values are means±SEM. *p<0.05, **p<0.01, ***p<0.001. Abbreviations: LoF, loss-of-function; MBOAT7, membrane-bound O-acyltransferase domain containing 7.

FIGS. 14A-G show hepatocyte MBOAT7 silencing after the development of hepatosteatosis shows evidence of upregulation of the cholesterol trafficking-induced TAZ pathway. Mice were fed the FPC diet for 8 weeks to induce hepatosteatosis and then injected with AAV8-H1-shMboat7 or AAV8-H1-shControl (shCtrl) virus. The mice were continued on the FPC diet for an additional 10 days. Livers were assayed for (A) phospho-S89-TAZ and total TAZ, (B) uncleaved and cleaved SREBP2, (C) mRNAs of sterol-responsive genes, (D) phosphatidylserine content, (E) diacylglycerol content, and (F) phospho-S133-CREB and total CREB. The values for all graphs are means±SEM. n=4 mice/group and values are means±SEM. *p<0.05, **p<0.01, ***p<0.001. (G) Proposed PL-TAZ pathway linking MBOAT7 LoF to liver fibrosis in MASH. Due to impaired acylation, MBOAT7 LoF favors the conversion of PI to LPI. (Tanaka Y, Shimanaka Y, Caddeo A, Kubo T, Mao Y, Kubota T, et al. LPIAT1/MBOAT7 depletion increases triglyceride synthesis fueled by high phosphatidylinositol turnover. Gut. 2021; 70: 180-93) However, consistent with the idea that MBOAT7 LoF promotes PI turnover, there is eventual conversion back to PI through a pathway involving AGPAT1 to form LPA and CDS2 to form CDP-DAG and then conversion of CD-DAG to PI. (Tanaka Y, Shimanaka Y, Caddeo A, Kubo T, Mao Y, Kubota T, et al. LPIAT1/MBOAT7 depletion increases triglyceride synthesis fueled by high phosphatidylinositol turnover. Gut. 2021; 70: 180-93.) This pathway leads to DAG formation, which can promote PC synthesis. The enzyme PSS1 converts PC into PS, which promotes ASTER-B/C-mediated transport of plasma membrane cholesterol to the cell interior, thereby triggering a TAZ-stabilizing pathway involving PKA, calcium, RhoA, and inhibition of TAZ phosphorylation (Wang X, Cai B, Yang X, Sonubi O O, Zheng Z, Ramakrishnan R, et al. Cholesterol stabilizes TAZ in hepatocytes to promote experimental non-alcoholic steatohepatitis. Cell Metab. 2020; 31: 969-986.e967). TAZ (with its cofactor TEAD) induces IHH, which is then secreted, leading to HSC activation and liver fibrosis (Wang X, Zheng Z, Caviglia J M, Corey K E, Herfel T M, Cai B, et al. Hepatocyte TAZ/WWTR1 promotes inflammation and fibrosis in nonalcoholic steatohepatitis. Cell Metab. 2016; 24: 848-62). Though not depicted here, TAZ/TEAD likely induces other genes that also contribute to MASH progression. Abbreviations: AGPAT1, 1-acylglycerol-3-phosphate O-acyltransferase 1; DAG, diacylglycerol; FPC, fructose-palmitate-cholesterol diet; IHH, Indian hedgehog; LoF, loss-of-function; LPA, lysophosphatidic acid; LPI, lysophosphatidylinositol; MASH, metabolic dysfunction-associated steatohepatitis; MBOAT7, membrane-bound O-acyltransferase domain containing 7; PI, phosphatidylinositol.

FIG. 15 shows MBOAT7 is suppressed in mice with FPC diet-induced steatohepatitis and MASH. Mice were fed the FPC diet for 8 weeks to cause hepatosteatosis or 16 weeks to cause early MASH. MBOAT7 immunofluorescence staining and quantification of MBOAT7 in the livers are shown (scale bar=50 μm). n=5-8 mice/group, and values are means+SEM. ****p<0.0001.

FIGS. 16A-L show additional characterization of mice treated with AAV8-TBG-Mboat7 to restore MBOAT7 in hepatocytes after the development of hepatosteatosis. The mice from the experiment in FIG. 9 were assayed for (A) body weight, (B) liver:body weight ratio, (C) fasting blood glucose, (D) liver CK19 by immunohistochemistry (scale bars=100 μm), (E) Mmp1 and Timp2 mRNA, (F) triglycerides, and (G-I) TNFα, IL6, and IL-1β by immunohistochemistry (scale bars=100 μm). (J) The mice were assayed for plasma ALT activity. (K-L) The livers were assayed for Pnpla3 and Hsd17b13 mRNA. n=6-8 mice/group, and the values are means±SEM. In panels D, and G-I, the data are expressed as percent area. *p<0.05, ***p<0.001.

FIGS. 17A-L show additional characterization of mice treated with AAV8-H1-shMboat7 to silence MBOAT7 in hepatocytes after the development of hepatosteatosis. The mice from the experiment in FIG. 10 were assayed for (A) body weight, (B) liver:body weight ratio, (C) fasting blood glucose, (D) liver CK19 by immunohistochemistry (scale bars=100 μm), (E) Mmp1 and Timp2 mRNA, (F) triglycerides, and (G-I) TNFα, IL6, and IL-1β by immunohistochemistry (scale bars=100 μm). (J) The mice were assayed for plasma ALT activity. (K-L) The livers were assayed for Pnpla3 and Hsd17b13 mRNA. n=6-7 mice/group, and the values are means±SEM. In panels D, and G-I, the data are expressed as percent area. **p<0.01.

FIGS. 18A-M show additional characterization of Mboat7 silencing in primary mouse hepatocytes, lipid profiles of control and Mboat7-silenced AML12 cells, and documentation of gene knockdown in Ptdss1-KD and Agpat1-KD AML12 cells. (A) Mouse primary hepatocytes transfected with siMboat7 or control siRNA were treated for 6 h with 400 μM oleic acid and then assayed for TAZ and MBOAT7, with densitometric quantification (n=3 technical replicates/group). (B) Liver specimens from obese individuals with the indicated genotypes at rs641738 (see FIG. 12D) were assayed for MBOAT7 mRNA, expressed relative to the CC value (n=46 CC, 48 TC, 29 TT). (C—H) Control and Mboat7-silenced AML12 cells were assayed by LC-MS/MS for (C) lysophosphatidylinositol (LPI), (D) phosphatidylinositol (PI), (E) phosphatidylcholine (PC), (F) phosphatidylglycerol (PG), (G) cholesteryl ester (CE), and (H) phosphatidylserine (PS). (I) Ptdss1 mRNA in wildtype vs. Ptdss1-knockdown (KD) AML12 cells, expressed relative to the wildtype values. (J) Control (Ctrl) and Ptdss1-knockdown (KD) AML12 cells were assayed by LC-MS/MS for phosphatidylserine (PS) and cholesteryl ester (CE). (K-L) Control and Mboat7-silenced AML12 cells were assayed by LC-MS/MS for phosphatidic acid (PA) and diacylglycerol (DAG). (M) Agapt1 mRNA in wildtype vs. Agpat1-KD AML12 cells, expressed relative to the wildtype values. Values are means±SEM. *p<0.05, **p<0.01, ***p<0.001, ****p<0.0001

FIGS. 19A-M show additional characterization of mice treated with AAV8-H1-shMboat7 to silence MBOAT7 in hepatocytes for 10 days after the development of hepatosteatosis. (A-M) The mice from the experiment in FIG. 14 were assayed for (A) liver Mboat7 mRNA, (B) body weight, (C) liver:body weight ratio, (D) fasting blood glucose; (E) lipid-droplet area (scale bar, 100 μm), (F) 4-HNE+ area (scale bar=200 μm), (G) F4/80+ area (scale bar=100 m), (H) Pnpla3 mRNA, (I) Hsd17b13 mRNA, (J) Picrosirius red+ area (scale bar=200 μm), (K) mRNAs associated with HSC activation, (L) cleaved SREBP1 via immunoblot, and (M) phosphatidylserine (PS). n=4 mice/group, and the values are means SEM. *p<0.05.

FIG. 20 shows the subject characteristics for FIG. 12C.

FIG. 21 shows the subject characteristics for FIG. 12D.

FIG. 22 shows the siRNA sequences used in this study.

FIG. 23 shows the primers used for qPCR.

### DETAILED DESCRIPTION

All patent applications, published patent applications, issued and granted patents, texts, and literature references cited in this specification are hereby incorporated herein by reference in their entirety to more fully describe the state of the art to which the present disclosed subject matter pertains.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The present disclosure provides compositions and methods for treating and/or preventing NASH fibrosis or metabolic dysfunction-associated steatohepatitis (MASH). The disclosure further provides methods for treating and/or preventing NASH fibrosis or MASH in patients possessing a specific risk variant, rs641738. The compositions and methods disclosed herein can be used as disease modifying therapies to enable prevention or treatment of NASH fibrosis or MASH and related disorders earlier in disease progression and improve clinical outcomes. The disclosure is based, at least in part, on the discovery, that the rs641738 variant of the MBOAT7 gene in humans, which results in low expression of MBOAT7 can lead to the development of NASH fibrosis or MASH, and that this result is due the gene's critical role in negative regulation of the hepatocyte TAZ signaling pathway, the pathway discovered to be critical in the development of NASH fibrosis or MASH. Using a personalized medicine approach, the inventors developed methods of treatment or preventing NASH fibrosis or MASH comprising either overexpressing MBOAT7 or silencing TAZ, Indian hedgehog (IHH), PS synthase 1 (PSS1), Aster-B/C, 1-acylglycerol-3-phosphate O-acyltransferase (AGPAT1), 1-CDP-diacylglycerol (DAG) synthase-2 (CDS2), diacylglycerol (DAG) or phosphatidylinositol (PI) among subjects who possess the risk variant rs641738.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

The term “contacting” or “contact” as used herein in connection with contacting a population of cells, e.g. a population of hepatic cells includes, subjecting the cells to an appropriate culture media which comprises the indicated compound or agent. Where the cell population is in vivo, “contacting” or “contact” includes administering the compound or agent in a pharmaceutical composition to a subject via an appropriate administration route such that the compound or agent contacts the cell population in vivo.

For in vivo methods, a therapeutically effective amount of a compound described herein can be administered to a subject. Methods of administering compounds to a subject are known in the art and easily available to one of skill in the art.

The terms “treatment,” “treating,” “treat,” “therapy,” “therapeutic,” and the like are used herein to refer generally to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, and includes: (a) preventing the disease or symptom from occurring in a subject which may be predisposed to the disease or symptom, may or may not be diagnosed as having it; (b) inhibiting the disease symptom, i.e., arresting its development; or (c) relieving the disease symptom, i.e., causing regression of the disease or symptom.

The expression “therapeutically effective amount” refers to an amount of an agent disclosed herein, that is effective for preventing, ameliorating, treating or delaying the onset of a disease or condition.

As used herein the term “variant” covers nucleotide or amino acid sequence variants which have about 99%, about 98%, about 97%, about 96%, about 95%, about 94%, about 93%, about 92%, about 91%, about 90%, about 85%, about 80%, about 75%, about 70%, or about 65% nucleotide identity, or about 99%, about 98%, about 97%, about 96%, about 95%, about 94%, about 93%, about 92%, about 91%, about 90%, about 85%, about 80%, about 75%, about 70%, or about 65% amino acid identity, including but not limited to variants comprising conservative, or non-conservative substitutions, deletions, insertions, duplications, or any other modification.

As used herein MASH refers to metabolic dysfunction-associated steatohepatitis. MASH was previously referred to as NASH. References to MASH herein are intended to refer to MASH as well as the prior nomenclature of NASH and references to NASH are intended to refer to MASH.

The pharmaceutical compositions of the inventions can be administered to any animal that can experience the beneficial effects of the agents of the invention. Such animals include humans and non-humans such as primates, pets and farm animals.

The present invention also comprises pharmaceutical compositions comprising the agents disclosed herein. Routes of administration and dosages of effective amounts of the pharmaceutical compositions comprising the agents are also disclosed. The agents of the present invention can be administered in combination with other pharmaceutical agents in a variety of protocols for effective treatment of disease.

Pharmaceutical compositions of the present invention are administered to a subject in a manner known in the art. The dosage administered will be dependent upon the age, health, and weight of the recipient, kind of concurrent treatment, if any, frequency of treatment, and the nature of the effect desired. One may administer the viral vectors, RNAi, shRNA or other inhibitors, or related compound in a local rather than systemic manner, for example, via injection of directly into the desired target site, often in a depot or sustained release formulation. Furthermore, one may administer the composition in a targeted drug delivery system, for example, in a liposome coated with a tissue-specific antibody, targeting, for example, the liver, and more specifically hepatocytes. The liposomes will be targeted to and taken up selectively by the desired tissue. Also included in a targeted drug delivery system is nanoparticle specific liver delivery of the viral vectors, RNAi, shRNA or other inhibitors, or compound, alone or in combination with similar compounds. A summary of various delivery methods and techniques of siRNA administration in ongoing clinical trials is provided in Zuckerman and Davis 2015; Nature Rev. Drug Discovery, Vol. 14: 843-856, December 2015.

One of ordinary skill in the art will appreciate that a method of administering pharmaceutically effective amounts of the pharmaceutical compositions of the invention to a patient in need thereof, can be determined empirically, or by standards currently recognized in the medical arts. The agents can be administered to a patient as pharmaceutical compositions in combination with one or more pharmaceutically acceptable excipients. It will be understood that, when administered to a human patient, the total daily usage of the agents of the pharmaceutical compositions of the present invention will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

Dosaging can also be administered in a patient-specific manner to provide a predetermined concentration of the agents in the blood, as determined by techniques accepted and routine in the art.

### I. Compositions for Regulating the Hepatocyte TAZ Pathway Expression.

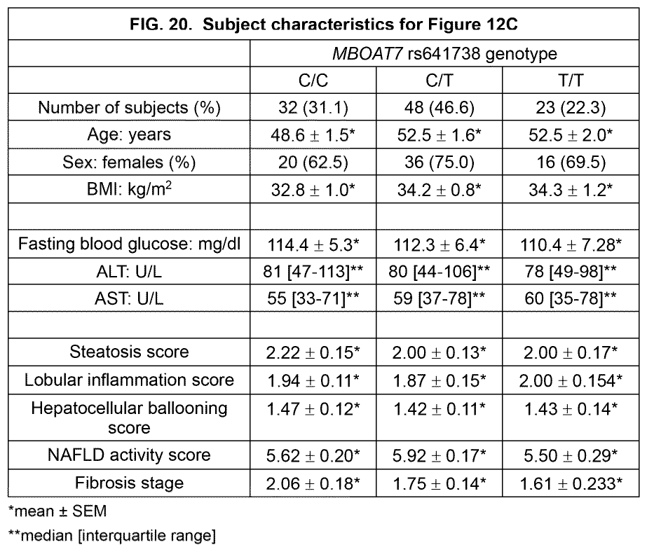

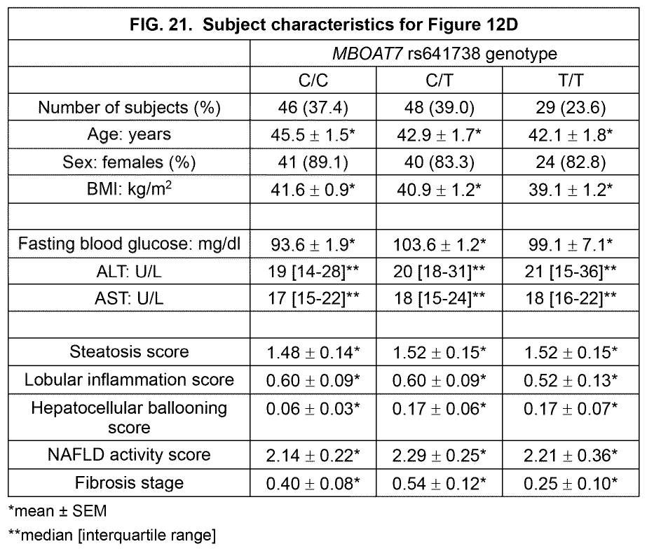

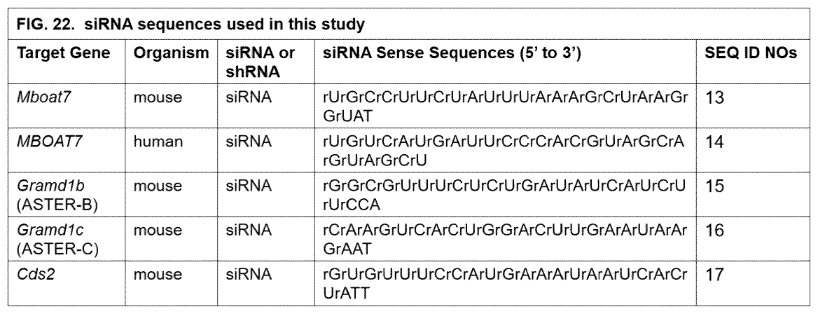

In various embodiments, the present application discloses compositions for decreasing the hepatic TAZ pathway that plays a critical role in the development of NASH fibrosis or MASH. In various embodiments, the present application discloses compositions for decreasing a cholesterol trafficking pathway that plays a critical role in the development of NASH fibrosis or MASH. In various embodiments, the present application discloses compositions for decreasing a PL metabolism pathway that plays a critical role in the development of NASH fibrosis or MASH. In various embodiments, the present application discloses a composition that inhibits TAZ expression or function. In various embodiments, the present application discloses a composition that inhibits Indian hedgehog (IHH) expression or function. In various embodiments, the present application discloses a composition that inhibits PS synthase-1 (PSS1) expression or function. In various embodiments, the present application discloses a composition that inhibits Aster-B/C expression or function. In various embodiments, the present application discloses a composition that inhibits 1-acylglycerol-3-phosphate O-acyltransferase (AGPAT1) expression or function. In various embodiments, the present application discloses a composition that inhibits CDP-diacylglycerol (DAG) synthase-2 (CDS2) expression or function. In various embodiments, the present application discloses a composition that inhibits diacylglycerol (DAG) expression or function. In various embodiments, the present application discloses a composition that inhibits phosphatidylinositol (PI) expression or function. In various embodiments, the present application discloses a composition comprising TAZ siRNA. In various embodiments, the present application discloses a composition comprising an Aster-B/C siRNA or a CDS2 siRNA. See FIG. 22. In various embodiments, the composition comprises the amino acid sequence of SEQ ID NO: 15 or 16. In various embodiments, the composition comprises the amino acid sequence of SEQ ID NO: 17.

In some embodiment, targeted gene expression can be reduced by several genome editing techniques such as RNAi (RNA interference), zinc finger nucleases (ZFNs), a TALE-effector domain nuclease (TALLEN), prime editing and base editing, CRISPR/Cas9 systems which are known in the art. In some embodiment, the CRISPR/Cas9 systems comprise a guide RNA (gRNA) or a single-molecule guide RNA (sgRNA). In some embodiment, the gRNA or sgRNA comprises a spacer sequence that is complementary to a portion of a nucleic acid sequence encoding TAZ. In some embodiment, the gRNA or sgRNA comprises a spacer sequence that is complementary to a portion of a nucleic acid sequence encoding IHH. In some embodiment, the gRNA or sgRNA comprises a spacer sequence that is complementary to a portion of a nucleic acid sequence encoding PSS1. In some embodiment, the gRNA or sgRNA comprises a spacer sequence that is complementary to a portion of a nucleic acid sequence encoding Aster-B/C. In some embodiment, the gRNA or sgRNA comprises a spacer sequence that is complementary to a portion of a nucleic acid sequence encoding AGPAT1. In some embodiment, the gRNA or sgRNA comprises a spacer sequence that is complementary to a portion of a nucleic acid sequence encoding CDS2. In some embodiment, the gRNA or sgRNA comprises a spacer sequence that is complementary to a portion of a nucleic acid sequence encoding DAG. In some embodiment, the gRNA or sgRNA comprises a spacer sequence that is complementary to a portion of a nucleic acid sequence encoding PI. In some embodiments, gRNA or sgRNA comprises SEQ ID NO: 11 or 12.

Inhibition of RNA encoding TAZ, IHH, PSS1 Aster-B/C, AGPAT1, CDS2, DAG and/or PI can effectively modulate the expression of these proteins. Inhibitors can include shRNAs encoding siRNAs, siRNA; interfering RNA or RNAi; dsRNA; RNA Polymerase III transcribed DNAs; ribozymes; GalNac-siRNA; GalNAc-Antisense Oligonucleotide (ASO) and antisense nucleic acids, which can be RNA, DNA, or an artificial nucleic acid.

Antisense oligonucleotides, including antisense DNA, RNA, and DNA/RNA molecules, act to directly block the translation of mRNA by binding to targeted mRNA and preventing protein translation. Antisense nucleotide sequences include, but are not limited to: morpholinos, 2′-O-methyl polynucleotides, DNA, RNA and the like.

siRNA comprises a double stranded structure containing from about 15 to about 50 base pairs, for example from about 21 to about 25 base pairs, and having a nucleotide sequence identical or nearly identical to an expressed target gene or RNA within the cell. The siRNA comprise a sense RNA strand and a complementary antisense RNA strand annealed together by standard Watson-Crick base-pairing interactions. The sense strand comprises a nucleic acid sequence which is substantially identical to a nucleic acid sequence contained within the target miRNA molecule. “Substantially identical” to a target sequence contained within the target mRNA refers to a nucleic acid sequence that differs from the target sequence by about 3% or less. The sense and antisense strands of the siRNA can comprise two complementary, single-stranded RNA molecules, or can comprise a single molecule in which two complementary portions are base-paired and are covalently linked by a single-stranded “hairpin” area.

The siRNA can be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribo-nucleotides. One or both strands of the siRNA can also comprise a 3′ overhang. As used herein, a 3′ overhang refers to at least one unpaired nucleotide extending from the 3′-end of a duplexed RNA strand. For example, the siRNA can comprise at least one 3′ overhang of from 1 to about 6 nucleotides (which includes ribonucleotides or deoxyribonucleotides) in length, or from 1 to about 5 nucleotides in length, or from 1 to about 4 nucleotides in length, or from about 2 to about 4 nucleotides in length. For example, each strand of the siRNA can comprise 3′ overhangs of dithymidylic acid (“TT”) or diuridylic acid (“uu”).

siRNA can be produced chemically or biologically, or can be expressed from a recombinant plasmid or viral vector. Methods for producing and testing dsRNA or siRNA molecules are known in the art. A short hairpin RNA (shRNA) encodes an RNA molecule with a tight hairpin turn that can be used to silence target gene expression via RNA interference (RNAi). Expression of shRNA in cells is typically accomplished by delivery of plasmids or through viral or bacterial vectors.

RNA polymerase III transcribed DNAs contain promoters, such as the U6 promoter. These DNAs can be transcribed to produce small hairpin RNAs in the cell that can function as siRNA or linear RNAs, which can function as antisense RNA. The CASP8, METRN, Kit and/or Stat3 inhibitor can comprise ribonucleotides, deoxyribonucleotides, synthetic nucleotides, or any suitable combination such that the target RNA and/or gene is inhibited. In addition, these forms of nucleic acid can be single, double, triple, or quadruple stranded

In various embodiments, the present application discloses compositions for enhancing MBOAT7 expression, a negative regulator of the TAZ pathway, a cholesterol trafficking pathway or a PL metabolism pathway. MBOAT7 expression may be enhanced using any known method in the art. For example, in various embodiments the composition is a vector encoding a gene for expressing MBOAT7. In various embodiments, the vector is a viral vector. In various embodiments, the viral vector is an AAV vector. In various embodiments, the viral vector is a vector that preferentially targets the liver or liver cells. In various embodiments the AAV is AAV 1, 2, 3, 4, 5, 6, 7, 8, 9, or 10. In various embodiments, the AAV is AAV8. In various embodiments, the present application discloses regulating the hepatic TAZ pathway, a cholesterol trafficking pathway or a PL metabolism pathway through the AAV-TBG-MBOAT7 vector as shown in FIG. 7. In various embodiments MBOAT7 is encoded by the nucleic acid sequence comprising SEQ ID NO: 1 or 2. In various embodiments, MBOAT7 comprises the amino acid sequence of SEQ ID NO: 3 or 4. In various embodiments, the composition is at least 75%, 80%, 85%, 90%, 95%, 96%, 97%, 98% or 99% identical to the nucleic acid sequence of SEQ ID NO: 1 or 2. In various embodiments, the composition is at least 75%, 80%, 85%, 90%, 95%, 96%, 97%, 98% or 99% identical to the amino acid sequence of SEQ ID NO: 3 or 4.

In some embodiments, the composition increases expression of MBOAT7. In some embodiments, the expression MBOAT7 is increased in liver cells of the subject. The expression of MBOAT7 may be increased by at least about 5% to about 95%, e.g., about 5%, 10%, 15%, 20%, 30%, 40%, 50%, 60%, 70%, 80%, 90%, 95%, relative to corresponding expression of MBOAT7 in a subject suffering from MASH.

In various embodiments, the composition comprises MBOAT7 mRNA. In various embodiments, the composition comprises an mRNA nanoparticle. In various embodiments, the composition comprises an mRNA enclosed in a lipid nanoparticle (LNP). Various LNPs that could be used include an ionizable cationic lipid (pKa in the range of 6.0-6.5, proprietary to Acuitas Therapeutics), PEG-lipid, or GM3-LNP. In various embodiments, the mRNA nanoparticle comprises MBOAT7 mRNA. In various embodiments MBOAT7 is encoded by the nucleic acid sequence comprising SEQ ID NO: 1 or 2. In various embodiments, MBOAT7 comprises the amino acid sequence of SEQ ID NO: 3 or 4. In various embodiments the composition comprises an RNA nanoparticle. In various embodiments, the RNA nanoparticle comprises TAZ siRNA. In various embodiments, the RNA nanoparticle comprises IHH siRNA. In various embodiments, the RNA nanoparticle comprises PSS1 siRNA. In various embodiments, the RNA nanoparticle comprises Aster-B/C siRNA. In various embodiments, the RNA nanoparticle comprises SEQ ID NO: 15 or 16. In various embodiments, the RNA nanoparticle comprises SEQ ID NO: 17. In various embodiments, the RNA nanoparticle comprises AGPAT1 siRNA. In various embodiments, the RNA nanoparticle comprises CDS2 siRNA. In various embodiments, the RNA nanoparticle comprises DAG siRNA. In various embodiments, the RNA nanoparticle comprises PI siRNA.

H. Methods of Treating and/or Preventing Nonalcoholic Steatohepatitis (NASH) Fibrosis/Metabolic Dysfunction-Associated Steatohepatitis (MASH)

In certain aspects, described herein is a method for treating or preventing MASH in a subject in need thereof, comprising administering to the subject a composition targeting the hepatic TAZ pathway, a cholesterol trafficking pathway or a PL metabolism pathway.

In various embodiments, the present application discloses methods for treating or preventing NASH or MASH in a subject in need thereof, comprising administering to said patient a composition that increases MBOAT7 expression. MBOAT7 expression may be enhanced using any known method in the art. For example, in various embodiments the composition is a vector encoding a gene for expressing MBOAT7. In various embodiments, the vector is a viral vector. In various embodiments, the viral vector is an AAV vector. In various embodiments, the viral vector is a vector that preferentially targets the liver or liver cells. In various embodiments, the AAV is AAV 1, 2, 3, 4, 5, 6, 7, 8, 9, or 10. In various embodiments, the AAV is AAV8. In various embodiments the patient is a mammal. In various embodiments, the mammal is a human. In various embodiments, the human expresses the rs641738 variant. In various embodiments, the composition comprises MBOAT7 mRNA. In various embodiments, the composition comprises an mRNA nanoparticle. In various embodiments, the composition comprises an mRNA enclosed in a lipid nanoparticle (LNP). Various LNPs that could be used include an ionizable cationic lipid (pKa in the range of 6.0-6.5, proprietary to Acuitas Therapeutics), PEG-lipid, or GM3-LNP. In various embodiments MBOAT7 is encoded by the nucleic acid sequence comprising SEQ ID NO: 1 or 2. In various embodiments, MBOAT7 comprises the amino acid sequence of SEQ ID NO: 3 or 4. In various embodiments the composition comprises an RNA nanoparticle. In various embodiments, the RNA nanoparticle comprises TAZ siRNA. In various embodiments, the RNA nanoparticle comprises IHH siRNA. In various embodiments, the RNA nanoparticle comprises PSS1 siRNA. In various embodiments, the RNA nanoparticle comprises Aster-B/C siRNA. In various embodiments, the RNA nanoparticle comprises SEQ ID NO: 15 or 16. In various embodiments, the RNA nanoparticle comprises SEQ ID NO: 17. In various embodiments, the RNA nanoparticle comprises AGPAT1 siRNA. In various embodiments, the RNA nanoparticle comprises CDS2 siRNA. In various embodiments, the RNA nanoparticle comprises DAG siRNA. In various embodiments, the RNA nanoparticle comprises PI siRNA.

In various embodiments, the present application discloses methods for treating or preventing NASH in a subject in need thereof, comprising administering to said patient a composition that targets the hepatic TAZ pathway, a cholesterol trafficking pathway or a PL metabolism pathway. In various embodiments, the composition targets MBOAT7. In various embodiments, the method involves administering a composition that increases expression of MBOAT7. MBOAT7 expression may be enhanced using any known method in the art. For example, in various embodiments the composition is a viral vector encoding a gene for expressing MBOAT7. In various embodiments, the vector is a viral vector. In various embodiments, the viral vector is an AAV vector. In various embodiments, the viral vector is a vector that preferentially targets the liver or liver cells. In various embodiments the AAV is AAV 1, 2, 3, 4, 5, 6, 7, 8, 9, or 10. In various embodiments, the AAV is AAV8. In various embodiments, the patient is a mammal. In various embodiments, the mammal is a human. In various embodiments, the human expresses the rs641738 variant. In various embodiments, the composition comprises MBOAT7 mRNA. In various embodiments, the composition comprises an mRNA enclosed in a lipid nanoparticle (LNP). Various LNPs that could be used include an ionizable cationic lipid (pKa in the range of 6.0-6.5, proprietary to Acuitas Therapeutics), PEG-lipid, or GM3-LNP.

In various embodiments MBOAT7 is encoded by the nucleic acid sequence comprising SEQ ID NO: 1 or 2. In various embodiments, MBOAT7 comprises the amino acid sequence of SEQ ID NO: 3 or 4. In various embodiments, the composition is at least 75%, 80%, 85%, 90%, 95%, 96%, 97%, 98% or 99% identical to the nucleic acid sequence of SEQ ID NO: 1 or 2. In various embodiments, the composition is at least 75%, 80%, 85%, 90%, 95%, 96%, 97%, 98% or 99% identical to the amino acid sequence of SEQ ID NO: 3 or 4.

In some embodiments, the composition increases expression of MBOAT7. In some embodiments, the expression MBOAT7 is increased in liver cells of the subject. The expression of MBOAT7 may be increased by at least about 5% to about 95%, e.g., about 5%, 10%, 15%, 20%, 30%, 40%, 50%, 60%, 70%, 80%, 90%, 95%, relative to corresponding expression of MBOAT7 in a subject suffering from MASH.

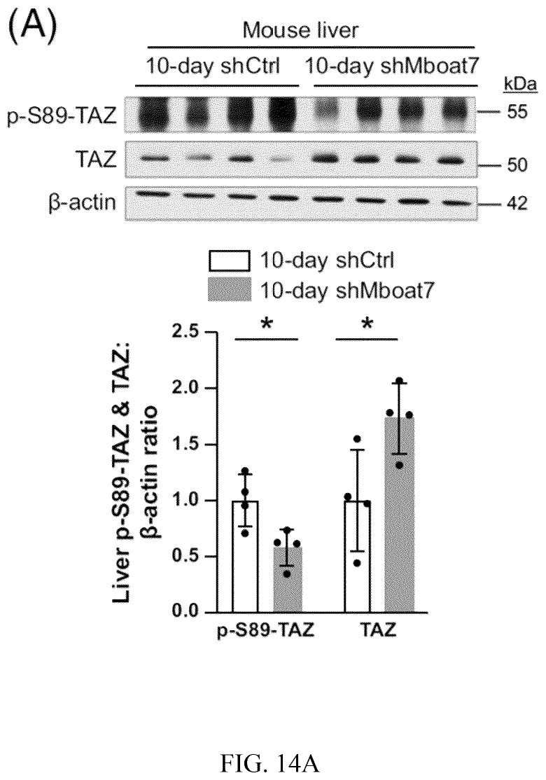

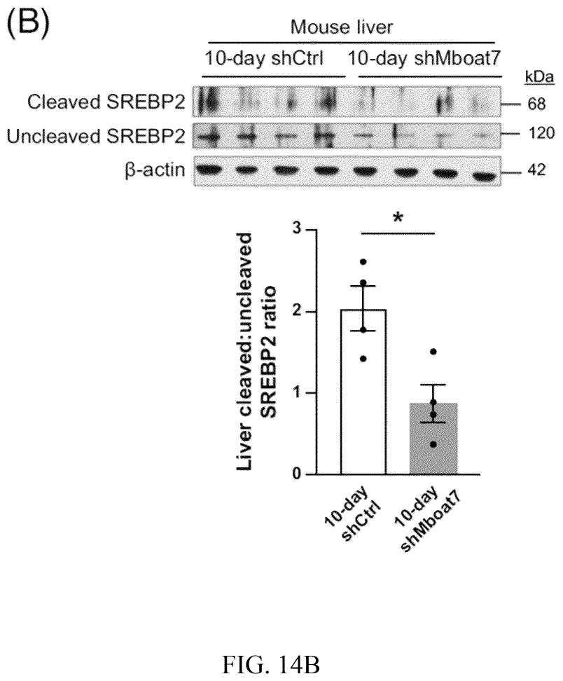

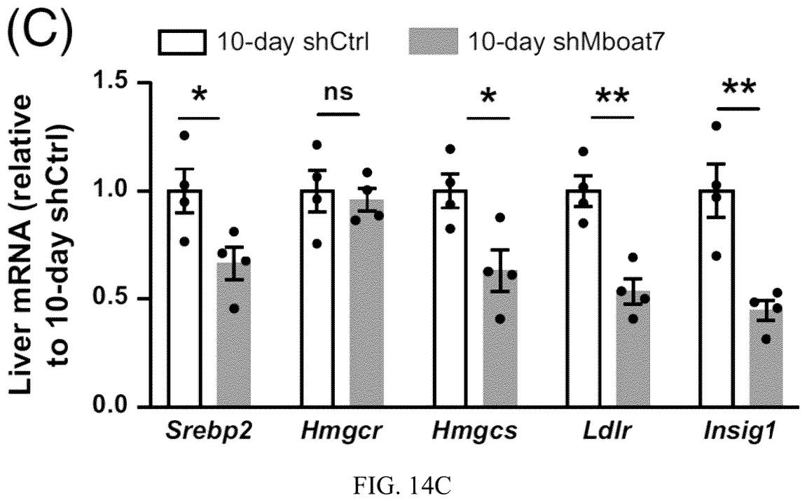

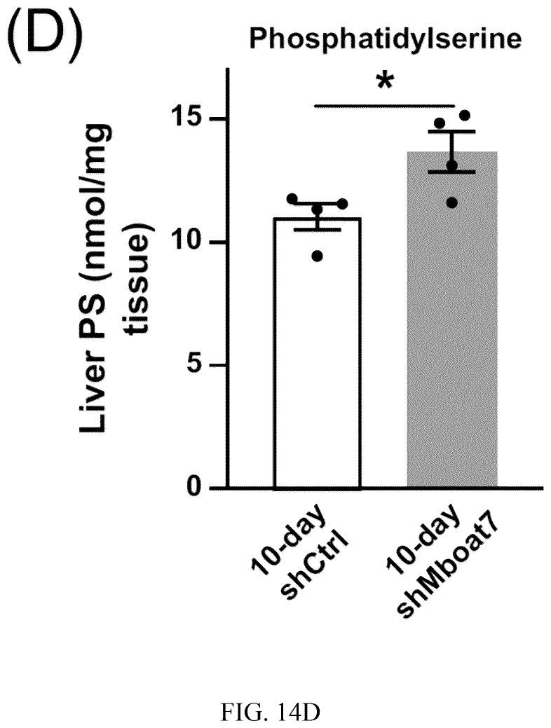

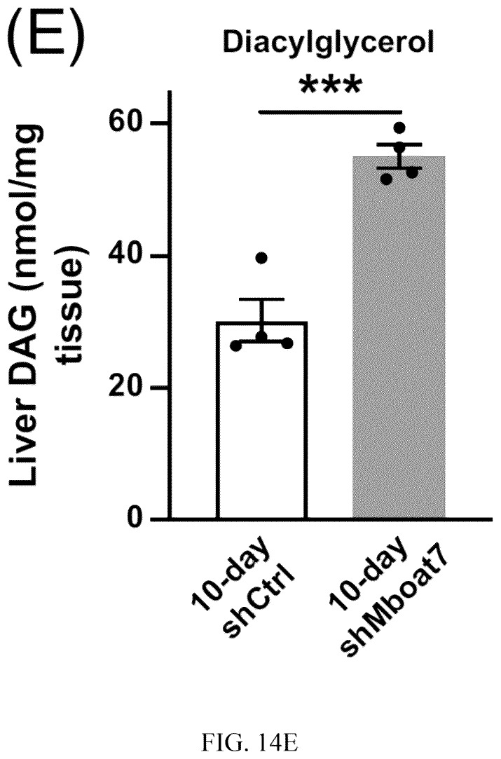

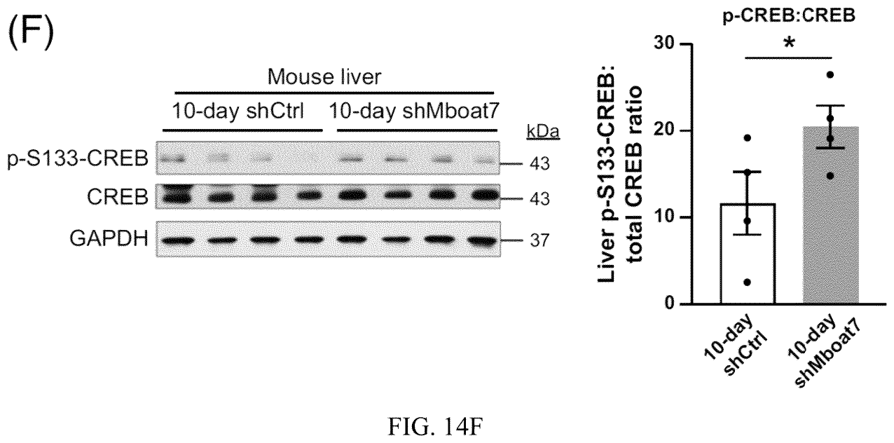

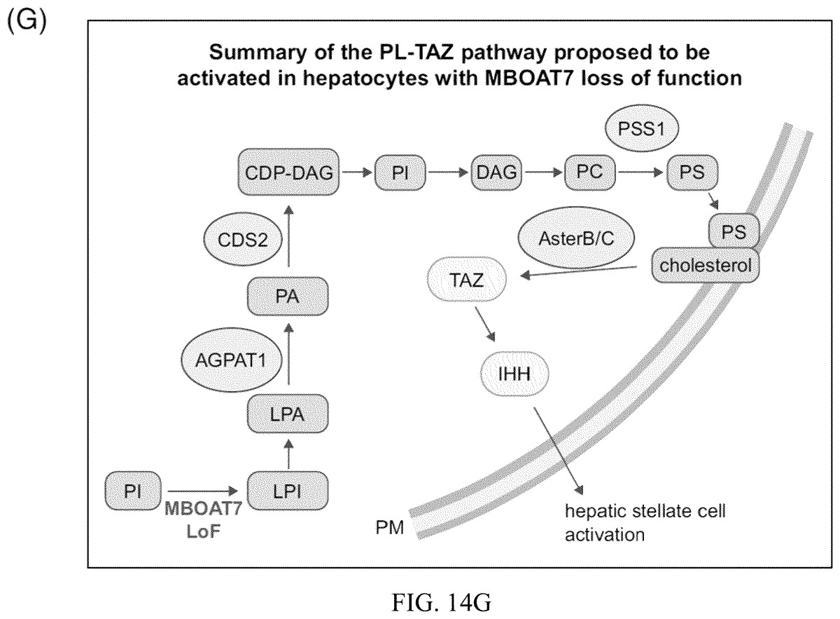

In some embodiments, the composition reduces or inhibits expression of one or more proteins or mRNAs disclosed in FIG. 14G. In some embodiments, the composition reduces or inhibits TAZ expression in the subject when compared to untreated subjects or to expression level of TAZ in the subject pre-treatment. In some embodiments, the composition reduces or inhibits IHH expression in the subject when compared to untreated subjects or to expression level of IHH in the subject pre-treatment. In some embodiments, the composition reduces or inhibits PSS1 expression in the subject when compared to untreated subjects or to expression level of PSS1 in the subject pre-treatment. In some embodiments, the composition reduces or inhibits Aster-B/C expression in the subject when compared to untreated subjects or to expression level of Aster-B/C in the subject pre-treatment. In some embodiments, the composition reduces or inhibits AGPAT1 expression in the subject when compared to untreated subjects or to expression level of AGPAT1 in the subject pre-treatment. In some embodiments, the composition reduces or inhibits CDS2 expression in the subject when compared to untreated subjects or to expression level of CDS2 in the subject pre-treatment. In some embodiments, the composition reduces or inhibits DAG expression in the subject when compared to untreated subjects or to expression level of DAG in the subject pre-treatment. In some embodiments, the composition reduces or inhibits PI expression in the subject when compared to untreated subjects or to expression level of PI in the subject pre-treatment. The expression of one or more proteins or mRNAs disclosed in FIG. 14G may be reduced by at least about 5% to about 95%, e.g., about 5%, 10%, 15%, 20%, 30%, 40%, 50%, 60%, 70%, 80%, 90%, 95%, relative to corresponding protein or mRNA expression in a subject suffering from MASH. The expression of TAZ, IHH, PSS1, Aster-B/C, AGPAT1, CDS2, DAG or PI may be reduced by at least about 5% to about 95%, e.g., about 5%, 10%, 15%, 20%, 30%, 40%, 50%, 60%, 70%, 80%, 90%, 95%, relative to corresponding expression of TAZ, IHH, PSS1, Aster-B/C, AGPAT1, CDS2, DAG or PI in a subject suffering from MASH. The expression of TAZ, IHH, PSS1, Aster-B/C, AGPAT1, CDS2, DAG or PI may be silenced relative to corresponding expression of TAZ, IHH, PSS1, Aster-B/C, AGPAT1, CDS2, DAG or PI in a subject suffering from MASH. In some embodiments, the expression of TAZ, IHH, PSS1, Aster-B/C, AGPAT1, CDS2, DAG or PI is reduced in liver cells of the subject.

In some embodiments, the composition comprises a TAZ small interfering ribonucleic acid (siRNA), a IHH siRNA, a PSS1 siRNA, a Aster-B/C siRNA, a AGPAT1 siRNA, a CDS2 siRNA, a DAG siRNA or a PI siRNA. In some embodiments, the Aster-B/C siRNA comprises SEQ ID No: 15 or 16. In some embodiments, the CDS2 siRNA comprises SEQ ID No: 17. In various embodiments, the siRNA comprises a nucleic acid sequence at least 75%, 80%, 85%, 90%, 95%, 96%, 97%, 98% or 99% identical to SEQ ID No: 15, 16 or 17.

In various embodiments the composition comprises an RNA nanoparticle. In various embodiments, the RNA nanoparticle comprises a TAZ small interfering ribonucleic acid (siRNA), a IHH siRNA, a PSS1 siRNA, a Aster-B/C siRNA, a AGPAT1 siRNA, a CDS2 siRNA, a DAG siRNA or a PI siRNA.

In some embodiments, the composition comprises a TAZ short-hairpin ribonucleic acid (shRNA), a IHH shRNA, a PSS1 shRNA, a Aster-B/C shRNA, a AGPAT1 shRNA, a CDS2 shRNA, a DAG shRNA or a PI shRNA. In some embodiments, the composition comprises a viral vector comprising a nucleic acid sequence encoding a TAZ short-hairpin ribonucleic acid (shRNA), a IHH shRNA, a PSS1 shRNA, a Aster-B/C shRNA, a AGPAT1 shRNA, a CDS2 shRNA, a DAG shRNA or a PI shRNA. In some embodiments, the viral vector is an adeno-associated vector (AAV). In various embodiments, the viral vector is a vector that preferentially targets the liver or liver cells. In various embodiments, the AAV is AAV 1, 2, 3, 4, 5, 6, 7, 8, 9, or 10 or variant thereof. In some embodiments, the viral vector is AAV8. In some embodiments, the viral vector is a hepatocyte-targeted AAV. In various embodiments, the shRNA comprises a nucleic acid sequence at least 75%, 80%, 85%, 90%, 95%, 96%, 97%, 98% or 99% identical to SEQ ID NOs: 15-17. See FIG. 22. In various embodiments, the vector is a viral vector comprising a nucleic acid encoding a TAZ short-hairpin ribonucleic acid (shRNA), a IHH shRNA, a PSS1 shRNA, a Aster-B/C shRNA, a AGPAT1 shRNA, a CDS2 shRNA, a DAG shRNA or a PI shRNA.

In some embodiments, the subject is a mammal. In some embodiments, the mammal is a human.

In some embodiments, the composition reduces or inhibits expression of one or more proteins or mRNAs disclosed in FIG. 14G. In some embodiments, the composition reduces or inhibits one or more mRNA expression of TAZ, IHH, PSS1, Aster-B/C, AGPAT1, CDS2, DAG or PI when compared to untreated subjects or to expression level of TAZ mRNA, IHH mRNA, PSS1 mRNA, Aster-B/C mRNA, AGPAT1 mRNA, CDS2 mRNA, DAG mRNA or PI mRNA, or level of TAZ protein, IHH protein, PSS1 protein, Aster-B/C protein, AGPAT1 protein, CDS2 protein, DAG protein or PI protein, or both levels of mRNA(s) and protein(s) in the subject pre-treatment. In some embodiments, the TAZ mRNA expression is reduced or inhibited in liver cells of the subject. In some embodiments, the IHH mRNA expression is reduced or inhibited in liver cells of the subject. In some embodiments, the PSS1 mRNA expression is reduced or inhibited in liver cells of the subject. In some embodiments, the ASTER-B/C mRNA expression is reduced or inhibited in liver cells of the subject. In some embodiments, the AGPAT1 mRNA expression is reduced or inhibited in liver cells of the subject. In some embodiments, the CDS2 mRNA expression is reduced or inhibited in liver cells of the subject. In some embodiments, the DAG mRNA expression is reduced or inhibited in liver cells of the subject. In some embodiments, the PI mRNA expression is reduced or inhibited in liver cells of the subject. In some embodiments, the TAZ protein expression is reduced or inhibited in liver cells of the subject. In some embodiments, the IHH protein expression is reduced or inhibited in liver cells of the subject. In some embodiments, the PSS1 protein expression is reduced or inhibited in liver cells of the subject. In some embodiments, the ASTER-B/C protein expression is reduced or inhibited in liver cells of the subject. In some embodiments, the AGPAT1 protein expression is reduced or inhibited in liver cells of the subject. In some embodiments, the CDS2 protein expression is reduced or inhibited in liver cells of the subject. In some embodiments, the DAG protein expression is reduced or inhibited in liver cells of the subject. In some embodiments, the PI protein expression is reduced or inhibited in liver cells of the subject. In some embodiments, one or more mRNA and/or protein expression of TAZ, IHH, PSS1, Aster-B/C, AGPAT1, CDS2, DAG or PI may be reduced by at least about 5% to about 95%, e.g., about 5%, 10%, 15%, 20%, 30%, 40%, 50%, 60%, 70%, 80%, 90%, 95%, relative to corresponding expression of TAZ, IHH, PSS1, Aster-B/C, AGPAT1, CDS2, DAG and/or PI in a subject suffering from MASH. The expression of TAZ, IHH, PSS1, Aster-B/C, AGPAT1, CDS2, DAG and/or PI may be silenced relative to corresponding to mRNA expression level of TAZ, IHH, PSS1, Aster-B/C, AGPAT1, CDS2, DAG or PI, protein expression level of TAZ, IHH, PSS1, Aster-B/C, AGPAT1, CDS2, DAG or PI, or both mRNA and protein expression levels of TAZ, IHH, PSS1, Aster-B/C, AGPAT1, CDS2, DAG or PI in a subject suffering from MASH.

In some embodiments, the expression of one or more proteins or mRNAs disclosed in FIG. 14G may be reduced using any known method in the art. TAZ, IHH, PSS1, Aster-B/C, AGPAT1, CDS2, DAG or PI expression may be reduced using any known method in the art. In some embodiments, the composition comprises a TAZ small interfering ribonucleic acid (siRNA), a IHH siRNA, a PSS1 siRNA, a Aster-B/C siRNA, a AGPAT1 siRNA, a CDS2 siRNA, a DAG siRNA or a PI siRNA. In some embodiments, the composition comprises one or more sequences encoding the small interfering ribonucleic acid (Aster-B/C siRNA or CDS siRNA) of SEQ ID No: 15, 16 or 17. For example, in various embodiments the composition comprises one or more siRNA sequences of SEQ ID No: 15, 16 or 17. In various embodiments, the siRNA comprises a nucleic acid sequence at least 75%, 80%, 85%, 90%, 95%, 96%, 97%, 98% or 99% identical to SEQ ID No: 15, 16 or 17. In some embodiments, the siRNA consists of a SEQ ID No: 15, 16 or 17.

In some embodiments, the composition comprises a TAZ short-hairpin ribonucleic acid (shRNA), a IHH shRNA, a PSS1 shRNA, a Aster-B/C shRNA, a AGPAT1 shRNA, a CDS2 shRNA, a DAG shRNA or a PI shRNA. In some embodiments, the Aster-B/C shRNA or the CDS2 shRNA comprises one or more SEQ ID No: 15, 16 or 17. In various embodiments, the shRNA comprises a nucleic acid sequence at least 75%, 80%, 85%, 90%, 95%, 96%, 97%, 98% or 99% identical to the SEQ ID No: 15, 16 or 17.

In some embodiments, the composition comprises a viral vector comprising a nucleic acid sequence encoding a shRNA. In some embodiments, the viral vector is an adeno-associated vector (AAV). In some embodiments, the viral vector is AAV8. In some embodiments, the composition comprises a viral vector encapsulating a nucleic acid sequence encoding a shRNA. In some embodiments, the viral vector is a hepatocyte-targeted AAV. In some embodiments, the composition reduces or inhibits expression of one or more proteins or mRNAs disclosed in FIG. 14G when compared to untreated subjects or the corresponding proteins or mRNAs expression level in the subject pre-treatment. In some embodiments, the composition reduces or inhibits TAZ, IHH, PSS1, Aster-B/C, AGPAT1, CDS2, DAG or PI expression when compared to untreated subjects or to expression level of TAZ, IHH, PSS1, Aster-B/C, AGPAT1, CDS2, DAG or PI in the subject pre-treatment. In some embodiments, TAZ, IHH, PSS1, Aster-B/C, AGPAT1, CDS2, DAG or PI expression is reduced or inhibited in liver cells of the subject. In various embodiments, the shRNA comprises a nucleic acid sequence at least 75%, 80%, 85%, 90%, 95%, 96%, 97%, 98% or 99% identical to the SEQ ID No: 15, 16 or 17. In some embodiments, the shRNA consists of a nucleic acid sequence of SEQ ID No: 15, 16 or 17. The expression of TAZ, IHH, PSS1, Aster-B/C, AGPAT1, CDS2, DAG and/or PI may be reduced by at least about 5% to about 95%, e.g., about 5%, 10%, 15%, 20%, 30%, 40%, 50%, 60%, 70%, 80%, 90%, 95%, relative to corresponding expression of TAZ, IHH, PSS1, Aster-B/C, AGPAT1, CDS2, DAG and/or PI in a subject suffering from MASH. The expression of TAZ, IHH, PSS1, Aster-B/C, AGPAT1, CDS2, DAG and/or PI may be silenced relative to corresponding expression of TAZ, IHH, PSS1, Aster-B/C, AGPAT1, CDS2, DAG and/or PI in a subject suffering from MASH.

In some embodiments, the composition comprises a guide RNA (gRNA) or a single-molecule guide RNA (sgRNA) comprising a spacer sequence that is complementary to a portion of a nucleic acid sequence encoding TAZ, IHH, PSS1, Aster-B/C, AGPAT1, CDS2, DAG or PI. In some embodiments, gRNA or sgRNA comprises SEQ ID NO: 11 or 12. In some embodiments, the composition comprises a viral vector comprising a nucleic acid sequence encoding a gRNA or sgRNA. In some embodiments, the viral vector is an adeno-associated vector (AAV). In some embodiments, the viral vector is AAV8. In some embodiments, the composition comprises a viral vector encapsulating a nucleic acid sequence encoding a gRNA or sgRNA. In some embodiments, the viral vector is a hepatocyte-targeted AAV.

In some embodiments, the composition comprises at least one of the gRNA or sgRNA sequences of SEQ ID No: 11 or 12. In various embodiments, the gRNA or sgRNA is at least 75%, 80%, 85%, 90%, 95%, 96%, 97%, 98% or 99% identical to SEQ ID No: 11 or 12.

In some embodiments, the hepatocyte-targeted nucleic acid comprises one or more gRNA or sgRNA sequences of SEQ ID No: 11 or 12. In various embodiments, the gRNA or sgRNA is at least 75%, 80%, 85%, 90%, 95%, 96%, 97%, 98% or 99% identical to SEQ ID No: 15, 16 or 17. In some embodiments, the siRNA consists of SEQ ID No: 11 or 12.

In some embodiments, the gRNA or the sgRNA is pre-complexed with a DNA endonuclease. In some embodiments, the DNA endonuclease is a Cas9 endonuclease.

In some embodiments, the composition reduces or inhibits expression of one or more proteins or mRNAs disclosed in FIG. 14G when compared to untreated subjects or to expression level of the corresponding proteins or mRNAs in the subject pre-treatment. In some embodiments, the composition reduces or inhibits expression of one or more of TAZ, IHH, PSS1, Aster-B/C, AGPAT1, CDS2, DAG or PI when compared to untreated subjects or to expression level of TAZ, IHH, PSS1, Aster-B/C, AGPAT1, CDS2, DAG or PI in the subject pre-treatment. In some embodiments, TAZ, IHH, PSS1, Aster-B/C, AGPAT1, CDS2, DAG or PI expression is reduced or inhibited in liver cells of the subject. The expression of TAZ, IHH, PSS1, Aster-B/C, AGPAT1, CDS2, DAG and/or PI may be reduced by at least about 5% to about 95%, e.g., about 5%, 10%, 15%, 20%, 30%, 40%, 50%, 60%, 70%, 80%, 90%, 95%, relative to corresponding expression of TAZ, IHH, PSS1, Aster-B/C, AGPAT1, CDS2, DAG and/or PI in a subject suffering from MASH. The expression of TAZ, IHH, PSS1, Aster-B/C, AGPAT1, CDS2, DAG and/or PI may be silenced relative to corresponding expression of TAZ, IHH, PSS1, Aster-B/C, AGPAT1, CDS2, DAG and/or PI in a subject suffering from MASH.

In some embodiments, the subject is a mammal. In some embodiments, the mammal is a human. In some embodiments, the composition is delivered systemically.

In some embodiments, a population of cells can be contacted with a compound or agent which, for example, includes subjecting the cells to an appropriate culture media which comprises the indicated compound or agent. Where the cell population is in vivo, contacting the cell population includes administering the compound or agent in a pharmaceutical composition to a subject via an appropriate administration route such that the compound or agent contacts the cell population in vivo.

For in vivo methods, a therapeutically effective amount of a compound described herein can be administered to a subject. Methods of administering compounds to a subject are known in the art and easily available to one of skill in the art.

As described herein, the methods of treatment described herein refer generally to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. Methods described herein covers any treatment of a disease in a subject, and includes: (a) preventing the disease or symptom from occurring in a subject which may be predisposed to the disease or symptom, may or may not be diagnosed as having it; (b) inhibiting the disease symptom, i.e., arresting its development; or (c) relieving the disease symptom, i.e., causing regression of the disease or symptom.

A therapeutically effective amount of an agent or composition disclosed herein, for example, is one that is effective for preventing, ameliorating, treating or delaying the onset of a disease or condition.

In various embodiments, the present application discloses methods for treating or preventing MASH in a subject in need thereof, comprising administering to said subject a composition that reduces or inhibits expression of proteins or mRNAs disclosed in FIG. 14G in hepatocytes. In various embodiments, the present application discloses methods for treating or preventing MASH in a subject in need thereof, comprising administering to said subject a composition that reduces or inhibits TAZ, IHH, PSS1, Aster-B/C, AGPAT1, CDS2, DAG or PI expression in hepatocytes. In some embodiments, the TAZ, IHH, PSS1, Aster-B/C, AGPAT1, CDS2, DAG or PI expression level is reduced in the subject as compared to an untreated subject suffering from MASH. In various embodiments, the present application discloses methods for treating or preventing MASH in a subject in need thereof, comprising administering to said subject a composition that reduces or inhibits TAZ, IHH, PSS1, Aster-B/C, AGPAT1, CDS2, DAG or PI expression compared to the TAZ, IHH, PSS1, Aster-B/C, AGPAT1, CDS2, DAG or PI expression before administration of the composition.

In some embodiments, the present application discloses methods for treating or preventing MASH in a subject in need thereof, comprising administering a composition that reduces hepatic stellate cell activation by reducing expression of one or more protein or mRNA, as disclosed in FIG. 14G, in hepatocytes. In some embodiments, the present application discloses methods for treating or preventing MASH in a subject in need thereof, comprising administering a composition that reduces hepatic stellate cell activation by reducing TAZ, IHH, PSS1, Aster-B/C, AGPAT1, CDS2, DAG or PI expression in hepatocytes. In some embodiments, the present application discloses methods for treating or preventing MASH in a subject in need thereof, comprising administering a composition that reduces hepatic stellate cell activation by reducing TAZ, IHH, PSS1, Aster-B/C, AGPAT1, CDS2, DAG or PI expression may be reduced using any known method in the art. For example, in various embodiments the composition comprises one or more siRNA sequences of SEQ ID No: 15, 16 or 17. In various embodiments, the siRNA comprises a nucleic acid sequence at least 75%, 80%, 85%, 90%, 95%, 96%, 97%, 98% or 99% identical to SEQ ID No: 15, 16 or 17. In some embodiments, the siRNA consists of SEQ ID No: 15, 16 or 17. In various embodiments, the present application discloses a composition comprising TAZ shRNA. In various embodiments, the present application discloses a composition comprising IHH shRNA. In various embodiments, the present application discloses a composition comprising PSS1 shRNA. In various embodiments, the present application discloses a composition comprising Aster-B/C shRNA. In various embodiments, the present application discloses a composition comprising AGPAT1 shRNA. In various embodiments, the present application discloses a composition comprising CDS2 shRNA. In various embodiments, the present application discloses a composition comprising DAG shRNA. In various embodiments, the present application discloses a composition comprising PI shRNA. For example, in various embodiments the composition is a vector encoding a shRNA wherein the shRNA comprises a nucleic acid sequence encoding the nucleic acid sequences of SEQ ID No: 15, 16 or 17. In various embodiments, the shRNA comprises a nucleic acid sequence at least 75%, 80%, 85%, 90%, 95%, 96%, 97%, 98% or 99% identical to the nucleic acid sequence as SEQ ID No: 15, 16 or 17. In some embodiments, the shRNA consists of SEQ ID No: 15, 16 or 17. In various embodiments, the vector is a viral vector containing a nucleic acid encoding a TAZ, IHH, PSS1, Aster-B/C, AGPAT1, CDS2, DAG or PI short-hairpin RNA (shRNA). In various embodiments, the viral vector is an AAV vector. In various embodiments, the viral vector is a vector that preferentially targets the liver or liver cells. In various embodiments, the AAV is AAV 1, 2, 3, 4, 5, 6, 7, 8, 9, or 10 or variant thereof. In various embodiments, the AAV is AAV8 or variant thereof. In some embodiments, the AAV, including the AAV8, is a hepatocyte-targeted AAV. In some embodiments, the composition comprises hepatocyte-targeted AAV8 containing a nucleic acid encoding TAZ, IHH, PSS1, Aster-B/C, AGPAT1, CDS2, DAG or PI short-hairpin RNA (shRNA). In various embodiments the subject is a mammal. In various embodiments, the mammal is a human. In various embodiments the composition comprises an RNA nanoparticle. In various embodiments, the RNA nanoparticle comprises a TAZ, IHH, PSS1, Aster-B/C, AGPAT1, CDS2, DAG or PI siRNA.

In various embodiments, the present application discloses methods for treating or preventing MASH in a subject in need thereof, comprising administering a composition that reduces hepatic stellate cell activation by reducing or inhibiting expression of one or more proteins or mRNAs disclosed in FIG. 14G. In various embodiments, the present application discloses methods for treating or preventing MASH in a subject in need thereof, comprising administering a composition that reduces hepatic stellate cell activation by reducing or inhibiting TAZ, IHH, PSS1, Aster-B/C, AGPAT1, CDS2, DAG and/or PI expression. The expression of TAZ, IHH, PSS1, Aster-B/C, AGPAT1, CDS2, DAG and/or PI may be reduced by at least about 5% to about 95%, e.g., about 5%, 10%, 15%, 20%, 30%, 40%, 50%, 60%, 70%, 80%, 90%, 95%, relative to corresponding expression of TAZ, IHH, PSS1, Aster-B/C, AGPAT1, CDS2, DAG and/or PI in a subject suffering from MASH. The expression of TAZ, IHH, PSS1, Aster-B/C, AGPAT1, CDS2, DAG and/or PI may be silenced relative to corresponding expression of TAZ, IHH, PSS1, Aster-B/C, AGPAT1, CDS2, DAG and/or PI in a subject suffering from MASH. In some embodiments, the expression of TAZ, IHH, PSS1, Aster-B/C, AGPAT1, CDS2, DAG and/or PI is reduced in liver cells of the subject.

### III. Pharmaceutical Compositions

In certain aspects, described herein is a composition for treating or preventing MASH comprising a hepatocyte-targeted nucleic acid targeting the hepatic TAZ pathway, cholesterol trafficking pathway or a PL metabolism pathway. In some embodiments, the composition comprises a viral vector comprising the nucleic acid. In some embodiments, the composition comprises a viral vector encapsulating the nucleic acid. In various embodiments, the viral vector is a vector that preferentially targets the liver or liver cells. In some embodiments, the viral vector is an AAV vector. In various embodiments, the AAV is AAV 1, 2, 3, 4, 5, 6, 7, 8, 9, or 10 or variant thereof. In some embodiments, the viral vector is AAV8. In some embodiments, the hepatocyte-targeted nucleic acid comprises N-acetyl galactosamine (GalNac). In some embodiments, the viral vector is a hepatocyte-targeted AAV.

In various embodiments, the present application discloses methods for treating or preventing NASH or MASH in a subject in need thereof, comprising administering to said patient a composition that increases MBOAT7 expression. MBOAT7 expression may be enhanced using any known method in the art. For example, in various embodiments the composition is a vector encoding a gene for expressing MBOAT7. In various embodiments, the vector is a viral vector. In various embodiments, the viral vector is an AAV vector. In various embodiments, the viral vector is a vector that preferentially targets the liver or liver cells. In various embodiments, the AAV is AAV 1, 2, 3, 4, 5, 6, 7, 8, 9, or 10. In various embodiments, the AAV is AAV8. In various embodiments the patient is a mammal. In various embodiments, the mammal is a human. In various embodiments, the human expresses the rs641738 variant. In various embodiments, the composition comprises MBOAT7 mRNA. In various embodiments, the composition comprises an mRNA nanoparticle. In various embodiments, the composition comprises an mRNA enclosed in a lipid nanoparticle (LNP). Various LNPs that could be used include an ionizable cationic lipid (pKa in the range of 6.0-6.5, proprietary to Acuitas Therapeutics), PEG-lipid, or GM3-LNP. In various embodiments MBOAT7 is encoded by the nucleic acid sequence comprising SEQ ID NO: 1 or 2. In various embodiments, MBOAT7 comprises the amino acid sequence of SEQ ID NO: 3 or 4. In various embodiments the composition comprises an RNA nanoparticle.

In various embodiments MBOAT7 is encoded by the nucleic acid sequence comprising SEQ ID NO: 1 or 2. In various embodiments, MBOAT7 comprises the amino acid sequence of SEQ ID NO: 3 or 4. In various embodiments, the composition is at least 75%, 80%, 85%, 90%, 95%, 96%, 97%, 98% or 99% identical to the nucleic acid sequence of SEQ ID NO: 1 or 2. In various embodiments, the composition is at least 75%, 80%, 85%, 90%, 95%, 96%, 97%, 98% or 99% identical to the amino acid sequence of SEQ ID NO: 3 or 4.

In some embodiments, the composition increases expression of MBOAT7. In some embodiments, the expression MBOAT7 is increased in liver cells of the subject. The expression of MBOAT7 may be increased by at least about 5% to about 95%, e.g., about 5%, 10%, 15%, 20%, 30%, 40%, 50%, 60%, 70%, 80%, 90%, 95%, relative to corresponding expression of MBOAT7 in a subject suffering from MASH.

In some embodiments, the composition reduces or inhibits expression of one or more proteins or mRNAs disclosed in FIG. 14G. In some embodiments, the composition reduces or inhibits expression of one or more of TAZ, IHH, PSS1, Aster-B/C, AGPAT1, CDS2, DAG or PI. In some embodiments, the expression of one or more of TAZ, IHH, PSS1, Aster-B/C, AGPAT1, CDS2, DAG or PI is reduced or inhibited in liver cells of the subject. The expression of TAZ, IHH, PSS1, Aster-B/C, AGPAT1, CDS2, DAG and/or PI may be reduced by at least about 5% to about 95%, e.g., about 5%, 10%, 15%, 20%, 30%, 40%, 50%, 60%, 70%, 80%, 90%, 95%, relative to corresponding expression of TAZ, IHH, PSS1, Aster-B/C, AGPAT1, CDS2, DAG and/or PI in a subject suffering from MASH. The expression of TAZ, IHH, PSS1, Aster-B/C, AGPAT1, CDS2, DAG and/or PI may be silenced relative to corresponding expression of TAZ, IHH, PSS1, Aster-B/C, AGPAT1, CDS2, DAG and/or PI in a subject suffering from MASH.

In some embodiments, composition comprises a guide ribonucleic acid (gRNA) or a single-molecule guide RNA (sgRNA) comprising a spacer sequence that is complementary to a portion of a nucleic acid sequence encoding TAZ, IHH, PSS1, Aster-B/C, AGPAT1, CDS2, DAG or PI. In some embodiments, gRNA or sgRNA comprises SEQ ID NO: 11 or 12.

In some embodiments, the composition comprises at least one of the gRNA or sgRNA sequences of SEQ ID No: 11 or 12. In various embodiments, the gRNA or sgRNA is at least 75%, 80%, 85%, 90%, 95%, 96%, 97%, 98% or 99% identical to SEQ ID No: 11 or 12.

In some embodiments, the hepatocyte-targeted nucleic acid comprises one or more gRNA or sgRNA sequences of SEQ ID No: 11 or 12. In various embodiments, the gRNA or sgRNA is at least 75%, 80%, 85%, 90%, 95%, 96%, 97%, 98% or 99% identical to SEQ ID No: 15, 16 or 17. In some embodiments, the siRNA consists of SEQ ID No: 11 or 12.

In some embodiments, the gRNA or the sgRNA is pre-complexed with a DNA endonuclease. In some embodiments, the DNA endonuclease is a Cas9 endonuclease.

In some embodiments, the composition comprises at least one of the small interfering ribonucleic acid (siRNA) sequences of SEQ ID No: 15, 16 or 17. In various embodiments, the siRNA is at least 75%, 80%, 85%, 90%, 95%, 96%, 97%, 98% or 99% identical to SEQ ID No: 15, 16 or 17.

In some embodiments, the hepatocyte-targeted nucleic acid comprises one or more small interfering ribonucleic acid (siRNA) sequences of SEQ ID No: 15, 16 or 17. In various embodiments, the siRNA is at least 75%, 80%, 85%, 90%, 95%, 96%, 97%, 98% or 99% identical to SEQ ID No: 15, 16 or 17. In some embodiments, the siRNA consists of SEQ ID No: 15, 16 or 17.

In some embodiments, the composition comprises an expression vector capable of targeting the hepatic TAZ pathway, cholesterol trafficking pathway or a PL metabolism pathway. In some embodiments, the expression vector capable of targeting the hepatic TAZ pathway, cholesterol trafficking pathway or a PL metabolism pathway encodes a TAZ, IHH, PSS1, Aster-B/C, AGPAT1, CDS2, DAG or PI short-hairpin RNA (shRNA).

The pharmaceutical compositions of the inventions can be administered to any animal that can experience the beneficial effects of the agents of the invention. Such animals include humans and non-humans such as primates, pets and farm animals.

The present invention also comprises pharmaceutical compositions comprising the agents disclosed herein. Routes of administration and dosages of effective amounts of the pharmaceutical compositions comprising the agents are also disclosed. The agents of the present invention can be administered in combination with other pharmaceutical agents in a variety of protocols for effective treatment of disease.

Pharmaceutical compositions of the present invention are administered to a subject in a manner known in the art. The dosage administered will be dependent upon the age, health, and weight of the recipient, kind of concurrent treatment, if any, frequency of treatment, and the nature of the effect desired. One may administer the viral vectors, siRNA, RNAi, shRNA or other inhibitors, or related compound in a local rather than systemic manner, for example, via injection of directly into the desired target site, often in a depot or sustained release formulation. Furthermore, one may administer the composition in a targeted drug delivery system, for example, in a liposome coated with a tissue-specific antibody, targeting, for example, the liver, and more specifically hepatocytes. The liposomes will be targeted to and taken up selectively by the desired tissue. Also included in a targeted drug delivery system is nanoparticle specific liver delivery of the viral vectors, siRNA, RNAi, shRNA or other inhibitors, or compound, alone or in combination with similar compounds. A summary of various delivery methods and techniques of siRNA administration in ongoing clinical trials is provided in Zuckerman and Davis 2015; Nature Rev. Drug Discovery, Vol. 14: 843-856, December 2015 the contents of which is hereby incorporated by reference in its entirety.

One of ordinary skill in the art will appreciate that a method of administering pharmaceutically effective amounts of the pharmaceutical compositions of the invention to a patient in need thereof, can be determined empirically, or by standards currently recognized in the medical arts. The agents can be administered to a patient as pharmaceutical compositions in combination with one or more pharmaceutically acceptable excipients. It will be understood that, when administered to a human patient, the total daily usage of the agents of the pharmaceutical compositions of the present invention will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

Dosaging can also be administered in a patient-specific manner to provide a predetermined concentration of the agents in the blood, as determined by techniques accepted and routine in the art.

### EXAMPLES

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield similar results.

### Example 1—Restoring Hepatocyte MBOAT7 In Vivo Lowers Liver Fibrosis

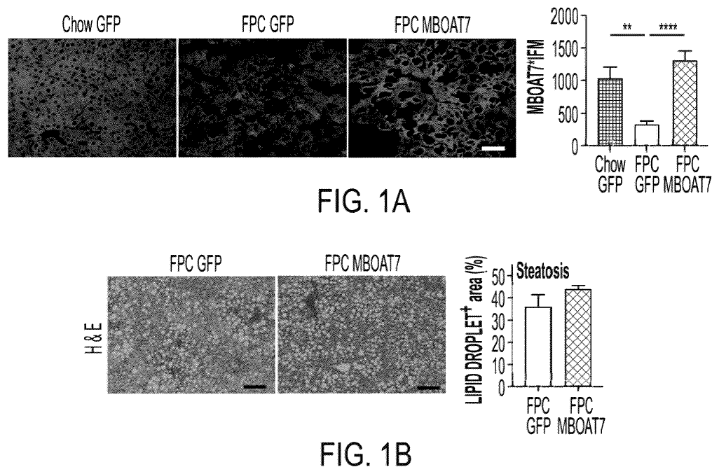

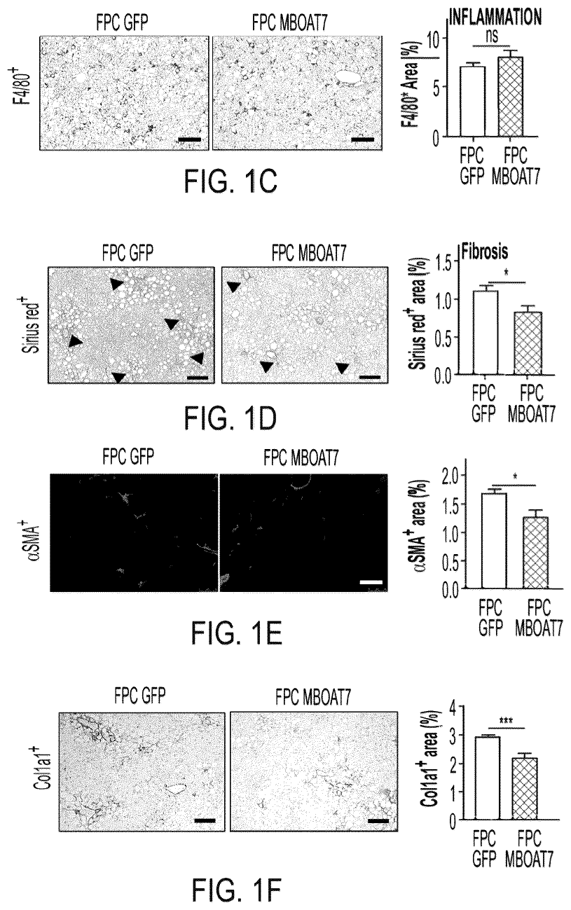

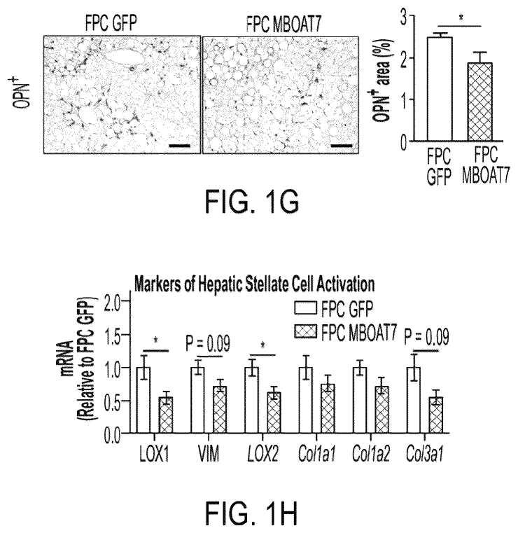

FIGS. 1A-H show restoring hepatocyte MBOAT7 in vivo lowers liver fibrosis. Male mice were fed a diet high in fructose, palmitate, and cholesterol (FPC) for 8 weeks to induce steatosis. The mice were then injected with adeno-associated viral (AAV) vector 8-TBG-MBOAT7 or AAV8-TBG-GFP control and continued on the FPC diet for an additional 8 weeks. FIG. 1A shows livers that were assayed for MBOAT7 expression by IFM and quantification. FIGS. 1B-C show improvements in fibrosis in AAV8-TBG-MBOAT7 mice were observed despite no change in steatosis and inflammation between groups. FIGS. 1D-G show AAV8-TBG-MBOAT7 treated mice had lower hepatic fibrosis compared to AAV8-TBG-GFP control mice as indicated by lower Sirius red staining, α-smooth muscle actin (αSMA) area, Col1a1 positive area, and OPN positive area. FIG. 1H shows AAV8-TBG-MBOAT7-treated mice had lower markers of hepatic stellate cell activation (Lox1 and Lox2 mRNAs) compared with AAV8-TBG-GFP controls. (Scale bars, 200 μm for B, C, and D, 50 μm for A, E, F and G. Means±SEM. N=6 mice/group. *P<0.05, **P<0.01, ***P<0.001, ****P<0.0001).

### Example 2—Inhibiting MBOAT7 Expression Triggers the Hepatic TAZ Pathway

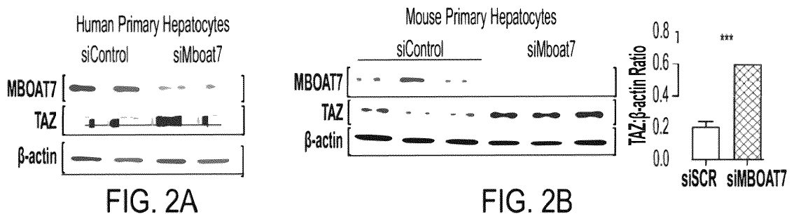

Primary mouse hepatocytes were transfected with siMBOAT7 siRNA. The target sequence of siMBOAT7 was UGC CUU CUA UUU AAA GCU AAG GUA T (SEQ ID NO: 8). The transfection protocol used is previously described (see, e.g., Wang et al., Cell Metab, 2020, 31(5):969-986; Wang et al., Cell Metab. 2016, 24(6):848-862). MBOAT7 mRNA expression (assayed via qPCR) and TAZ protein expression (assayed by Western blot) were then measured. Methods for qPCR and Western blot are previously described (see, e.g., Wang 2020; Wang et al., Cell Metab. 2016, 24(6):848-862 (“Wang 2016”)). As demonstrated in FIG. 2, silencing hepatocyte-MBOAT7 expression ex vivo increases hepatic TAZ. Hepatocyte TAZ is a major driver of NASH. FIG. 2A shows primary human hepatocytes treated with siMBOAT7 had greater TAZ protein compared to siControl-treated hepatocytes. FIG. 2B shows primary mouse hepatocytes treated with siMboat7 had greater TAZ protein compared to siControl-treated hepatocytes. For TAZ quantification data in FIG. 2B, means±SEM; n=3 biological samples; ***P<0.001.

### Example 3—Increase in TAZ Caused by Silencing Hepatocyte MBOAT7 is Dependent on Cholesterol Trafficking from the Plasma Membrane (PM)

siMBOAT7 induced expression of hepatic TAZ was found to be dependent on cholesterol trafficking from the plasma membrane (PM), which is relevant to NASH. AML12 cells were transfected with Scr or MBOAT7 siRNA and control or Aster B/C ASO, which blocks the trafficking of PM cholesterol from the cell surface to the interior of hepatocytes. The target sequence of siMBOAT7 was UGC CUU CUA UUU AAA GCU AAG GUA T (SEQ ID NO: 8) for mice and CUACUGCUACGUGGGAAUCAUGACA (SEQ ID NO: 9) for humans. The transfection protocol used is previously described (see, e.g., Wang 2020). Briefly, siRNA-Mediated Gene Silencing and Transfection Scrambled siRNA control and oligo-targeting siRNAs were transfected into AML12 or primary hepatocytes using Lipofectamine RNAiMAX (Life Technologies) at 40 nM of siRNA in 24-well plates following the manufacturer's instructions. 2×10⁵ cells at 30-40% confluence were incubated for 18 h with 0.5 ml of culture medium containing 1.5 ml Lipofectamine RNAiMAX and 20 pmol siRNA (10 pmol for ASTER B/C ASO, SEQ ID NOS: 7 and 8).

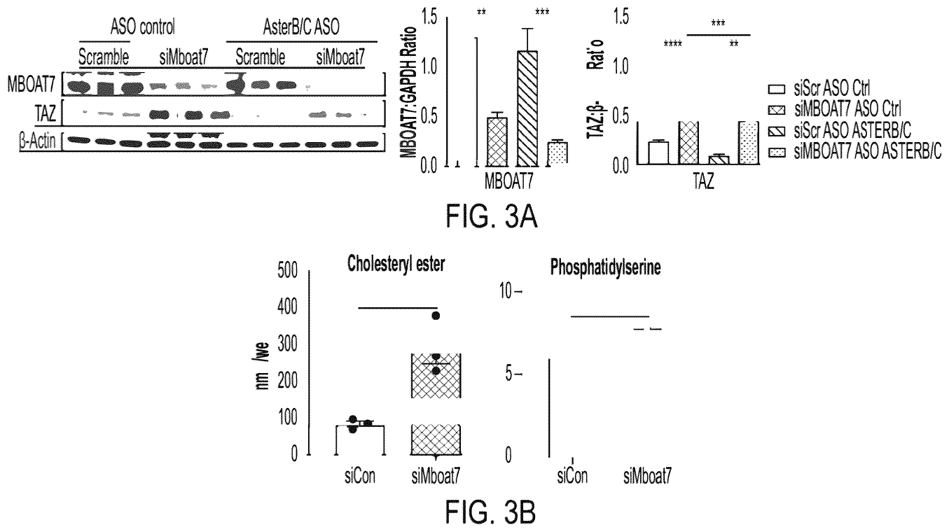

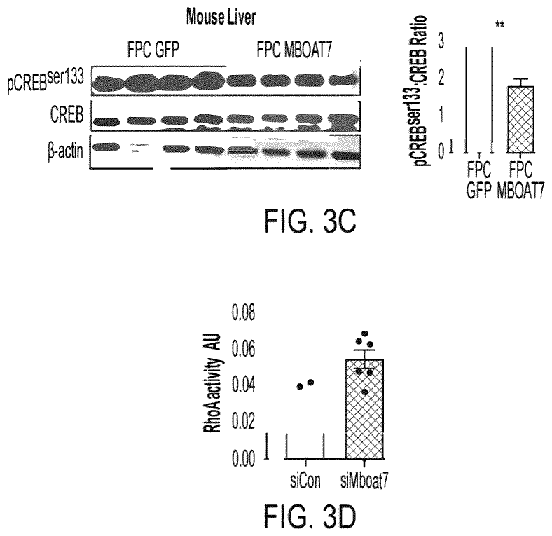

FIG. 3A shows that AML12 cells were transfected with Scr or MBOAT7 siRNA and control or Aster B/C ASO, which blocks the trafficking of PM cholesterol from the cell surface to the interior of hepatocytes. AML12 cells treated with siMBOAT7 had a greater increase in TAZ protein compared to control as in FIG. 2, and this increase was abrogated by Aster B/C-ASO. Values are means+SEM (n=3 biological samples). **P<0.01, ***P<0.001, ****P<0.0001.

FIG. 3B shows an increase in cholesterol ester, an intracellular cholesterol marker, and phosphatidylserine (PS) with MBOAT7 loss of function (LoF), and if this turns out to be important in the mechanism linking MBOAT7 LoF and the risk polymorphism (rs641738 C>T) to NASH, this data suggest that blocking hepatocyte PS synthesis in people at risk for NASH, e.g., using GalNAc-siPdtss1 might be beneficial. *P<0.05, n=3.

As shown in FIG. 3C, the livers of AAV8-TBG-MBOAT7-treated mice (see FIG. 1) had lower phospho-Creb^{ser133}:total CREB ratio, a marker of PKA activation. N=6 mice/group. **P<0.01. Conversely, MBOAT7 silencing activates the pathway, and increased RhoA activity in siMboat7-vs. Control-treated hepatocytes (FIG. 3D) was observed. Values are means SEM (n=6 biological samples). *P<0.05. These data, when considered with those above in mice and humans, adds support to the hypothesis that rs641738 C>T promotes NASH, at least in part, by activating a cholesterol-mediated pathway that upregulates the TAZ-IHH mechanism of NASH fibrosis. These data suggest that MBOAT7 loss-of-function triggers the TAZ pathway known to occur in NASH, i.e. by increasing PM cholesterol trafficking to the cell interior and activating phospho-Creb^{ser133} described by Wang et al. Cell Metabolism, 2020.

### Example 4—Restoring Hepatocyte MBOAT7 in NASH Lowers Hepatic TAZ Expression

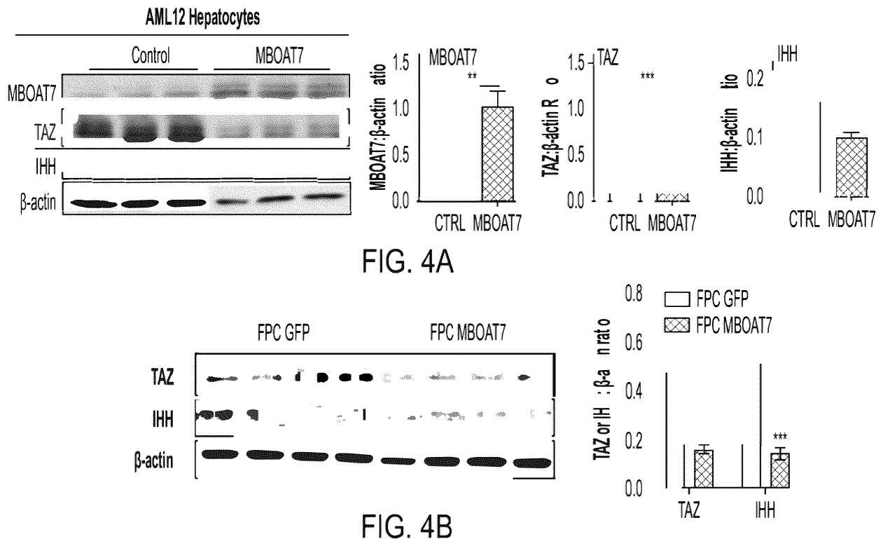

FIG. 4 shows that hepatocyte MBOAT7 decreases the pro-fibrotic TAZ-Indian hedgehog (IHH) pathway in hepatocytes and in the livers of FPC-fed mice. AML12 hepatocytes were transfected with GFP control or Mboat7 plasmid (SEQ ID NO: 5). FIG. 4A shows β-actin, TAZ, IHH and MBOAT7 proteins were analyzed by immunoblot. The MBOAT7/β-actin, TAZ/β-actin, and IHH/β-actin ratios are shown in the right graph of FIG. 4A. Means+SEM. N=3 biological samples; *P<0.05, **P<0.01, ***P<0.001. FIG. 4B shows that restoring hepatic-MBOAT7 lowers TAZ and its downstream pro-fibrotic factor IHH in male mice fed chow or FPC diet for 8 weeks to induce steatosis and then injected with AAV8-TBG-Mboat7 or AAV8-TBG-GFP control and continued on the FPC diet for an additional 8 weeks (see FIG. 1). FIG. 4B shows livers of AAV8-TBG-MBOAT7-treated mice from FIG. 1 had lower TAZ protein and lower IHH protein, which is the major TAZ target responsible for NASH. *P<0.05, ***P<0.001. (n=6-7 mice/group). Values are means±SEM.

### Example 5—Restoring MBOAT7 Expression in a Mouse Model of NASH Fibrosis

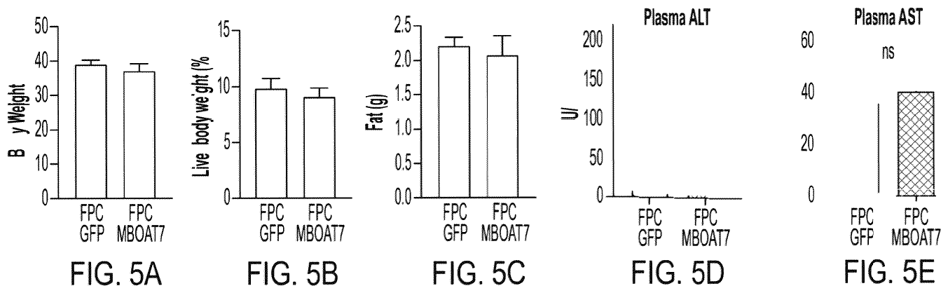

FIG. 5 shows that restoring hepatocyte MBOAT7 in NASH mice does not affect liver, body or fat pad weight and ALT/AST. In the experiment described in FIG. 1, body weight (FIG. 5A), liver:body weight ratio (FIG. 5B), fat pad weight (FIG. 5C), ALT (FIG. 5D), and AST (FIG. 5E) were not changed following AAV8-TBG-MBOAT7 treatment compared to GFP control. Means±SEM. n=6 mice/group.

### Example 6—Restoring MBOAT7 Expression Lowers Cholesterol Ester in NASH Mouse Livers

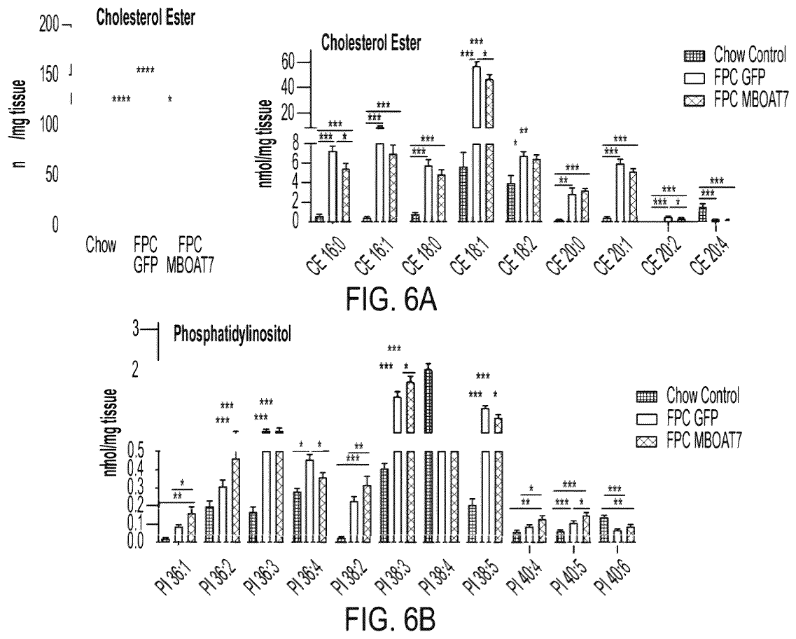

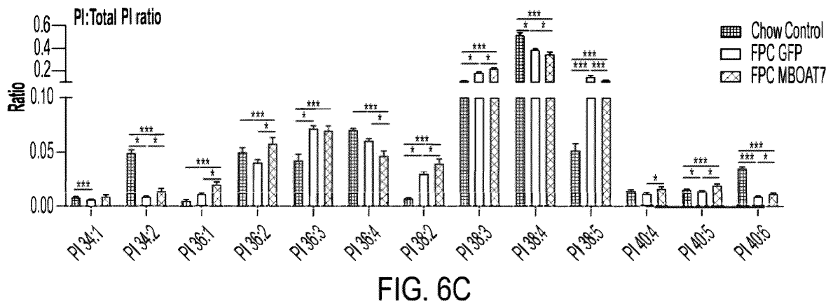

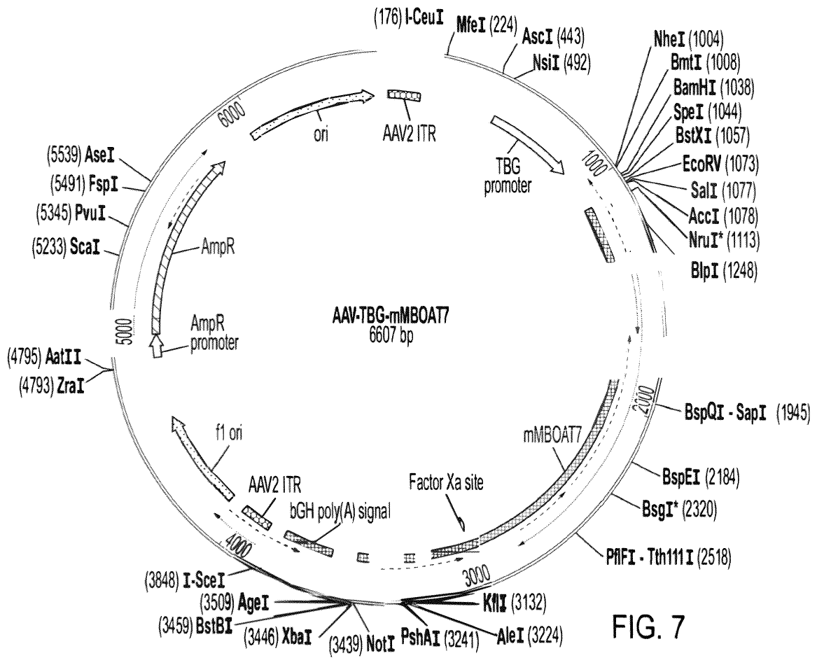

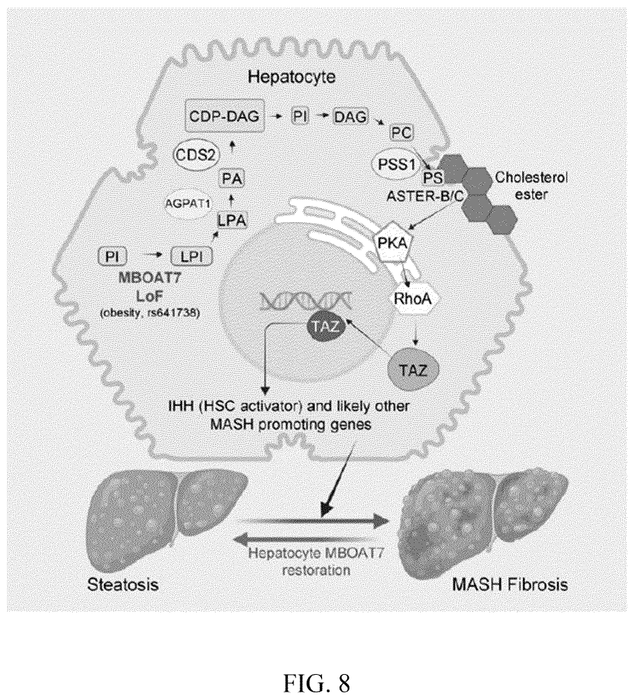

FIG. 6 shows restoring hepatocyte MBOAT7 lowers cholesterol ester and increases a few types of phosphatidylinositol in NASH mouse livers. FIG. 6A shows a decrease in total cholesterol ester and a spectrum of different cholesterol esters following AAV8-TBG-MBOAT7 treatment compared to GFP control. FIGS. 6B and 6C show an increase in PI and PI/total PI ratio following AAV8-TBG-MBOAT7 treatment compared to GFP control. Together with FIG. 3B, these data suggest MBOAT7 inhibited liver cholesterol is correlated with its enzyme activity. Values are means±SEM (n=6-7 mice/group). *P<0.05, **P<0.01, ***P<0.001, ****P<0.0001.

FIG. 7 shows a vector that created for the restoration experiment in FIG. 1. MBOAT7 is specifically expressed in hepatocytes through the TBG promotor. The vector is packed in adeno-associated viruses (AAV) serotype 8 with high liver specificity. The delivery method is intravenous injection, dose is 2×10¹¹ gc/mouse.

The following sequences will further exemplify the invention.

Mouse MBOAT7 mRNA sequence:
(SEQ ID NO: 1)
AGCTCTTTGCCCGGGGACCTCCTAGAAAGATCGCGACAGGCATCTTGCGGCAGACTGGTGTTCTGGGTAC
CTTTCTTGCGGTGCTGTAACTCGTACAGCCGCGGCTCTCGGGGCCTGGACCGCGCAGCCCTGCCGGCGCC
CTCCAGAACGGGCAGTGCGGGGGCGTGCTGAGCTGGGGAGGCGTGGCGCGAGCCGAGGCGGCCTCGAAAA
GGAGCTCCGCAGTCTTCTGGCCCACGGACGGTTCAGACCATGACACCCGAAGAATGGACATATCTAATGG
TCCTTCTTATCTCCATCCCTGTTGGCTTCCTCTTTAAGAAAGCTGGACCTGGGCTGAAGAGATGGGGGGC
AGCAGCTGTGGGCCTGGGGCTCACCTTATTCACCTGTGGCCCCCACAGTTTGCATTCTCTGATCACCATC
TTGGGAACCTGGGCCCTCATTCAGGCCCAGCCCTGCTCCTGCCATGCCCTGGCTCTTGCCTGGACCTTCT
CCTATCTCCTCTTCTTCCGAGCCCTCAGCCTGCTGGGCCTGCCCACTCCCACGCCCTTCACCAATGCTGT
CCAGCTGCTGTTGACACTGAAGTTGGTGAGTCTAGCTAGTGAAGTCCAGGATCTGCATCTGGCTCAGAGA
AAGGAAATAGCCTCCGGCTTCCACAAGGAGCCTACGCTGGGCCTCCTGCCTGAGGTCCCCTCTTTGATGG
AGACACTCAGCTATAGCTACTGTTACGTGGGAATCATGACAGGCCCATTCTTCCGCTACCGCACCTACCT
GGATTGGCTGGAACAGCCCTTCCCGGAAGCCGTGCCCAGCCTGAGGCCCCTGCTGCGCCGCGCCTGGCCA
GCCCCGCTCTTTGGCCTGCTCTTCCTGCTGTCCTCCCATCTCTTCCCACTGGAAGCTGTGCGTGAGGACG
CCTTCTACGCCCGCCCGCTGCCCACCCGCCTCTTCTACATGATCCCGGTCTTCTTCGCCTTCCGCATGCG
CTTCTACGTTGCCTGGATTGCGGCCGAGTGCGGTTGCATTGCCGCGGGCTTCGGGGCCTACCCTGTGGCT
GCCAAAGCCCGGGCCGGGGGCGGCCCCACCCTCCAATGCCCACCCCCTAGCAGTCCGGAGATTGCAGCTT
CCCTGGAGTATGACTATGAGACCATCCGTAACATCGACTGCTATGGCACAGACTTCTGCGTGCGTGTGCG
GGATGGCATGCGATACTGGAACATGACCGTGCAGTGGTGGCTGGCACAGTACATCTACAAGAGCGCACCT
TTCCGCTCCTACGTTTTGAGGAGTGCCTGGACCATGCTGTTGAGTGCCTACTGGCATGGCCTCCACCCTG
GTTACTACCTAAGCTTCATGACCATCCCGCTGTGCCTGGCTGCTGAGGGCTATTTGGAGTCAGCCTTGCG
GAGACACCTGAGCCCCGGGGGCCAGAAAGCCTGGGACTGGGTCCACTGGTTCCTGAAGATGCGTGCCTAC
GACTACATGTGCATGGGCTTTGTGCTCCTTTCCATGGCTGACACACTCCGGTACTGGGCCTCCATCTACT
TCTGGGTCCACTTTCTAGCCCTGGCTTGCTTGGGGCTGGGGCTGGTTTTGGGTGGGGGCAGCCCCAGCAA
GAGGAAGACACCATCCCAGGCCACCAGCAGCCAAGCGAAGGAAAAGCTCCGGGAAGAGTGAGCTCTGCTG
CATTGGCCTGCCTTCCAGTTCAAGCTTTTTTGGGAATTCCATGAACCAGGCTGTTTGTTTGGGGTTTTTG
TTTGTTTGTTTGTTTGTTTCCTTTACCCAGCAAGAATCCCTTGTTTGGCTAAGAGCCTGGAGAGGATCCC
CTCTTCCCAAATAATTCCTCTGCCTTCTATTTAAAGCTAAGGTATCCTTCTCTTGGGCTCTCTCAGCATC
TTGACCTTTTCAGACCTTCCTCTGCTAACATCAGGGTATTACTATCCACTCTTGAACCTATTATCTCTGC
AACAATCTTCAGATGTTCAAAAAGCCACACTTCCCAAAAATGCCCTTGCAGGGACCAGTGGTCATCTGGC
ATCTTAGACAGACTCCCAGTGGGTCCCCAGTATGGGGGCAGGAACTTCAGGGCCAGGTTCTGGGAGAGGG
GAGGGATAGCCTTCTTGTTTTCCTCTTTGTTTTTATCATCACACCAGTGTTTCAGAGACCATGGTCTTAC
ACATGCTGAAGGAGAAGCTAAAATGTGAGAAGCCCCAGGGGAGCTTGCTCTTACAGCAGCTTCTGCCTGA
GCCATTTCTGGGCTCCCCATACAACCTACCACCCAGTGTCATCCTTGGCCTGTGACAGGCCAGAATGTAT
AAAGCTTTCCCAATAAAGTGTTACACATGCA
Human MBOAT7 mRNA Sequence:
(SEQ ID NO: 2)
AGTGTGGACCTGGACTCGAATCCCGTTGCCGACTCGCGCTCTCGGCTTCTGCTCCGGGGCTTCTTCCCTG
CCCGCCCGGGGCCCTGACCGTGGCTTCTTCCCCGGCCTGATCTGCGCAGCCCGGCGGGCGCCCAGAAGGA
GCAGGCGGCGCGGGGGCGCGCTGGGCGGGGGAGGCGTGGCCGGAGCTGCGGCGGCAAGCGGGCTGGGACT
GCTCGGCCGCCTCCTGCCCGGCGAGCAGCTCAGACCATGTCGCCTGAAGAATGGACGTATCTAGTGGTTC
TTCTTATCTCCATCCCCATCGGCTTCCTCTTTAAGAAAGCCGGTCCTGGGCTGAAGAGATGGGGAGCAGC
CGCTGTGGGCCTGGGGCTCACCCTGTTCACCTGTGGCCCCCACACTTTGCATTCTCTGGTCACCATCCTC
GGGACCTGGGCCCTCATTCAGGCCCAGCCCTGCTCCTGCCACGCCCTGGCTCTGGCCTGGACTTTCTCCT
ATCTCCTGTTCTTCCGAGCCCTCAGCCTCCTGGGCCTGCCCACTCCCACGCCCTTCACCAATGCCGTCCA
GCTGCTGCTGACGCTGAAGCTGGTGAGCCTGGCCAGTGAAGTCCAGGACCTGCATCTGGCCCAGAGGAAG
GAAATGGCCTCAGGCTTCAGCAAGGGGCCCACCCTGGGGCTGCTGCCCGACGTGCCCTCCCTGATGGAGA
CACTCAGCTACAGCTACTGCTACGTGGGAATCATGACAGGCCCGTTCTTCCGCTACCGCACCTACCTGGA
CTGGCTGGAGCAGCCCTTCCCCGGGGCAGTGCCCAGCCTGCGGCCCCTGCTGCGCCGCGCCTGGCCGGCC
CCGCTCTTCGGCCTGCTGTTCCTGCTCTCCTCTCACCTCTTCCCGCTGGAGGCCGTGCGCGAGGACGCCT
TCTACGCCCGCCCGCTGCCCGCCCGCCTCTTCTACATGATCCCCGTCTTCTTCGCCTTCCGCATGCGCTT
CTACGTGGCCTGGATTGCCGCCGAGTGCGGCTGCATTGCCGCCGGCTTTGGGGCCTACCCCGTGGCCGCC
AAAGCCCGGGCCGGAGGCGGCCCCACCCTCCAATGCCCACCCCCCAGCAGTCCGGAGAAGGCGGCTTCCT
TGGAGTATGACTATGAGACCATCCGCAACATCGACTGCTACAGCACAGATTTCTGCGTGCGGGTGCGCGA
TGGCATGCGGTACTGGAACATGACGGTGCAGTGGTGGCTGGCGCAGTATATCTACAAGAGCGCACCTGCC
CGTTCCTATGTCCTGCGGAGCGCCTGGACCATGCTGCTGAGCGCCTACTGGCACGGCCTCCACCCGGGCT
ACTACCTGAGCTTCCTGACCATCCCGCTGTGCCTGGCTGCCGAGGGCCGGCTGGAGTCAGCCCTGCGGGG
GCGGCTGAGCCCAGGGGGCCAGAAGGCCTGGGACTGGGTGCACTGGTTCCTGAAGATGCGCGCCTATGAC
TACATGTGCATGGGCTTCGTGCTGCTCTCCTTGGCCGACACCCTTCGGTACTGGGCCTCCATCTACTTCT
GTATCCACTTCCTGGCCCTGGCAGCCCTGGGGCTGGGGCTGGCTTTAGGTGGGGGCAGCCCCAGCCGGCG
GAAGGCAGCATCCCAGCCCACCAGCCTTGCCCCGGAGAAGCTCCGGGAGGAGTAAGCTGTCACGACGCTC
CCTCTGCCAGCTGGTCCCGGGAATTCTGTGAACCAGGCTGCTGTCTCCTCCCCAGAAAGAGTCCTTACCT
TGGAGAGGGTCCTGGAGAGAATTTCCTCTTCCCCAGCTAAATACCCTGCCTGCAACTGAAGCAGACCCGG
GGGTGTCCTCCCTGCCCTCTGCCCAGAGGCCACCTCCACTCCTACAAAATCAAAGTATTGTCCAGACAAG
AGTCACTGGCCCCTGCTCCAGCTTCTGGGTATCCAGAGAGCACTGCACTTCCCCAAAACGGAAGGGGCCC
CTGGGCAGTGGGTTTTGGGCAAATTCCCTTTCTTTGCATCCACAATGTGGGGTCGGAGCTTGGGGGCAGG
TCCTGGGAGTGGGAAGCCTCTTCCTTGTGTCTTTCGCTCCACTTTTAGCTCATCGCACCAATATTGCAGA
CTTGGAAGGAAGCATAAGCTTCCCATTTCACAAAGGGGAAACTGAGGTGCGGGTGCGCGGGCCTGGGGAC
GGCCGTCCCATGGCTTCCATCTGAGCCACCTCGGGACCCCAGCACTCCTGGCGCCCTCTTCTCATCGCTT
GGCCTATGACAGGTCACCGTGTGTAAATCTTTCCCAATAAAGTGTTGCACAAAG
Mouse MBOAT7 Protein sequence:
(SEQ ID NO: 3)
MTPEEWTYLMVLLISIPVGFLFKKAGPGLKRWGAAAVGLGLTLFTCGPHSLHSLITILGTWALIQAQPCS
CHALALAWTFSYLLFFRALSLLGLPTPTPFTNAVQLLLTLKLVSLASEVQDLHLAQRKEIASGFHKEPTL
GLLPEVPSLMETLSYSYCYVGIMTGPFFRYRTYLDWLEQPFPEAVPSLRPLLRRAWPAPLFGLLFLLSSH
LFPLEAVREDAFYARPLPTRLFYMIPVFFAFRMRFYVAWIAAECGCIAAGFGAYPVAAKARAGGGPTLQC
PPPSSPEIAASLEYDYETIRNIDCYGTDFCVRVRDGMRYWNMTVQWWLAQYIYKSAPFRSYVLRSAWTML
LSAYWHGLHPGYYLSFMTIPLCLAAEGYLESALRRHLSPGGQKAWDWVHWFLKMRAYDYMCMGFVLLSMA
DTLRYWASIYFWVHFLALACLGLGLVLGGGSPSKRKTPSQATSSQAKEKLREE
Human MBOAT7 Protein sequence:
(SEQ ID NO: 4)
MSPEEWTYLVVLLISIPIGFLFKKAGPGLKRWGAAAVGLGLTLFTCGPHTLHSL VTILGTWALIQAQPCS
CHALALAWTFSYLLFFRALSLLGLPTPTPFTNAVQLLLTLKLVSLASEVQDLHLAQRKEMASGFSKGPTL
GLLPDVPSLMETLSYSYCYVGIMTGPFFRYRTYLDWLEQPFPGAVPSLRPLLRRAWPAPLFGLLFLLSSH
LFPLEAVREDAFYARPLPARLFYMIPVFFAFRMRFYVAWIAAECGCIAAGFGAYPVAAKARAGGGPTLQC
PPPSSPEKAASLEYDYETIRNIDCYSTDFCVRVRDGMRYWNMTVQWWLAQYIYKSAPARSYVLRSAWTML
LSAYWHGLHPGYYLSFLTIPLCLAAEGRLESALRGRLSPGGQKAWDWVHWFLKMRAYDYMCMGFVLLSLA
DTLRYWASIYFCIHFLALAALGLGLALGGGSPSRRKAASQPTSLAPEKLREE
MBOAT7 Plasmid sequence:
(SEQ ID NO: 5)
GGGACCTCCTAGAAAGATCGCGACAGGCATCTTGCGGCAGACTGGTGTTCTGGGTACCTTTCTTGCGGTG
CTGTAACTCGTACAGCCGCGGCTCTCGGGGCCTGGACCGCGCAGCCCTGCCGGCGCCGTCCAGAACGGGC
AGTGCGGGGGCGTGCTGAGCTGGGGAGGCGTGGCGCGAGCCGAGGCGGCCTCGAAAAGGAGCTCCGCAGT
TTTCTGGCCCACGGACGGTTCAGACCATGACACCCGAAGAATGGACATATCTAATGGTCCTTCTTATCTC
CATCCCTGTTGGCTTCCTCTTTAAGAAAGCTGGACCTGGGCTGAAGAGATGGGGGGCAGCAGCTGTGGGC
CTGGGGCTCACCTTATTCACCTGTGGCCCCCACAGTTTGCATTCTCTGATCACCATCTTGGGAACCTGGG
CCCTCATTCAGGCCCAGCCCTGCTCCTGCCATGCCCTGGCTCTTGCCTGGACCTTCTCCTATCTCCTCTT
CTTCCGAGCCCTCAGCCTGCTGGGCCTGCCCACTCCCACGCCCTTCACCAATGCTGTCCAGCTGCTGTTG
ACACTGAAGTTGGTGAGTCTAGCTAGTGAAGTCCAGGATCTGCATCTGGCTCAGAGAAAGGAAATAGCCT
CCGGCTTCCACAAGGAGCCTACGCTGGGCCTCCTCCCTGAGGTCCCCTCTTTGATGGAGACACTCAGCTA
TAGCTACTGTTACGTGGGAATCATGACAGGCCCATTCTTCCGCTACCGCACCTACCTGGATTGGCTGGAA
CAGCCCTTCCCGGAAGCCGTGCCCAGCCTGAGGCCCCTGCTGCGCCGCGCCTGGCCAGCCCCGCTCTTTG
GCCTGCTCTTCCTGCTGTCCTCCCATCTCTTCCCACTGGAAGCTGTGCGTGAGGACGCCTTCTACGCCCG
CCCGCTGCCCACCCGCCTCTTCTACATGATCCCGGTCTTCTTCGCCTTCCGCATGCGCTTCTACGTTGCC
TGGATTGCGGCCGAGTGCGGTTGCATTGCCGCGGGCTTCGGGGCCTACCCTGTGGCTGCCAAAGCCCGGG
CCGGGGGCGGCCCCACCCTCCAATGCCCACCCCCTAGCAGTCCGGAGATTGCAGCTTCCCTGGAGTATGA
CTATGAGACCATCCGTAACATCGACTGCTATGGCACAGACTTCTGCGTGCGTGTGCGGGATGGCATGCGA
TACTGGAACATGACCGTGCAGTGGTGGCTGGCACAGTACATCTACAAGAGCGCACCTTTCTGCTCCTACG
TTTTGAGGAGTGCCTGGACCATGCTGTTGAGTGCCTACTGGCATGGCCTCCACCCTGGTTACTACCTAAG
CTTCATGACCATCCCGCTGTGCCTGGCTGCTGAGGGCTATTTGGAGTCAGCCTTGCGGAGACACCTGAGC
CCCGGGGGCCAGAAAGCCTGGGACTGGGTCCACTGGTTCCTGAAGATGCGTGCCTACGACTACATGTGCA
TGGGCTTTGTGCTCCTTTCCATGGCTGACACACTCCGGTACTGGGCCTCCATCTACTTCTGGGTCCACTT
TCTAGCCCTGGCCTGCTTGGGGCTGGGGCTGGTTTTGGGTGGGGGCAGCCCCAGCAAGAGGAAGACACCA
TCCCAGGCTACCAGCAGCCAAGCGAAGGAAAAGCTCCGGGAAGAGTGAGCTCTGCTGCATTGGCCTGCCT
TCCAGTTCAAGCTTTTTTGGGAATTCCATGAACCAGGCTGTTTGTTTGGGGTTTTTGTTTGTTTGTTTGT
TTGTTTCCTTTACCCAGCAAGAATCCCTTGTTTGGCTAAGAGCCTGGAGAGGATTCCCTCTTCCCAAATA
ATTCCTCTGCCTTCTATTTAAAGCTAAGGTATCCTTCTCTTGGGCTCTCTCAGCATCTTGACCTTTTCAG
ACCTTCCTCTGCTAACATCAGGGTATTACTATCCACTCTTGAACCTATTATCTCTGCAACAATCTTCAGA
TGTTCAAAAAGCCACACTTCCCAAAAATGCCCTTGCAGGGACCAGTGGTCATCTGGCATCTTAGACAGAC
TCCCAGTGGGTCCCCAGTATGGGGGCAGGAACTTCAGGGCCAGGTTCTGGGAGAGGGGAGGGATAGCCTT
CTTGTTTTCCTCTTTGTTTTTATCATCACACCAGTGTTTCAGAGACCATGGTCTTACACATGCTGAAGGA
GAAGCTAAAATGTGAGAAGCCCCAGGGGAGCTTGCTCTTACAGCAGCTTCTGCCTGAGCCATTTCTGGGC
TCCCCATACAACCTACCACCCAGTGTCATCCTTGG
ASTER-B ASO:
(SEQ ID NO: 6)
mGmGmCmGmUTTCTCTGATATCATmCmUmUmCmCmA
ASTER-C ASO:
(SEQ ID NO: 7)
mCmAmAmGmUCACTGGACTTGAATmAmAmGmAmAmU
2′-O-Methyl bases are represented with a lower case “m” in front of each base.

### Example 7—Restoring MBOAT7 Expression Lowers Cholesterol Ester in NASH Mouse Livers

The common genetic variant rs641738 C>T is a risk factor for metabolic dysfunction-associated steatotic liver disease and metabolic dysfunction-associated steatohepatitis (MASH), including liver fibrosis, and is associated with decreased expression of the phospholipid-remodeling enzyme MBOAT7 (LPIAT1). However, whether restoring MBOAT7 expression in established metabolic dysfunction-associated steatotic liver disease dampens the progression to liver fibrosis and, importantly, the mechanism through which decreased MBOAT7 expression exacerbates MASH fibrosis remain unclear.

Approach and Results: Hepatocyte MBOAT7 restoration in mice with diet-induced steatohepatitis slows the progression to liver fibrosis. Conversely, when hepatocyte-MBOAT7 was silenced in mice with established hepatosteatosis, liver fibrosis but not hepatosteatosis was exacerbated. Mechanistic studies revealed that hepatocyte-MBOAT7 restoration in MASH mice lowered hepatocyte-TAZ (WWTR1), which is known to promote MASH fibrosis. Conversely, hepatocyte-MBOAT7 silencing enhanced TAZ upregulation in MASH. Finally, changes in hepatocyte phospholipids due to MBOAT7 loss-of-function promote a cholesterol trafficking pathway that upregulates TAZ and the TAZ-induced profibrotic factor Indian hedgehog (IHH). As evidence for relevance in humans, the livers of individuals with MASH carrying the rs641738-T allele had higher hepatocyte nuclear TAZ, indicating higher TAZ activity and increased IHH mRNA.

Conclusions: This invention provides evidence for a novel mechanism linking MBOAT7-LoF to MASH fibrosis, adds new insight into an established genetic locus for MASH, and, given the druggability of hepatocyte TAZ for MASH fibrosis, suggests a personalized medicine approach for subjects at increased risk for MASH fibrosis due to inheritance of variants that lower MBOAT7.

### Introduction

Metabolic dysfunction-associated steatohepatitis (MASH), previously referred to as NASH, is emerging as the leading cause of chronic liver disease. (Younossi Z M, Golabi P, Paik J M, Henry A, Van Dongen C, Henry L. The global epidemiology of nonalcoholic fatty liver disease (NAFLD) and nonalcoholic steatohepatitis (NASH): A systematic review. Hepatology. 2023; 77:1335-47). The disease begins with metabolic dysfunction-associated steatotic liver disease (MASLD), and ˜20% of individuals with MASLD then develop MASH with liver inflammation, injury, and, most importantly, fibrosis. (Angulo P, Kleiner D E, Dam-Larsen S, Adams L A, Bjornsson E S, Charatcharoenwitthaya P, et al. Liver fibrosis, but no other histologic features, is associated with long-term outcomes of patients with nonalcoholic fatty liver disease. Gastroenterology. 2015; 149:389-397.e310; Wree A, Broderick L, Canbay A, Hoffman H M, Feldstein A E. From NAFLD to NASH to cirrhosis-new insights into disease mechanisms. Nat Rev Gastroenterol Hepatol. 2013; 10: 627-36; Bohinc B N, Diehl A M. Mechanisms of disease progression in NASH: New paradigms. Clin Liver Dis. 2012; 16:549-65) There has been only one recently FDA-approved drug for MASH fibrosis, (Ray K. Resmetirom proves positive for NASH with liver fibrosis. Nat Rev Gastroenterol Hepatol. 2024; 21:218) due in large part to an incomplete understanding of the mechanisms of liver fibrosis, which is the main contributor to liver-related mortality in MASH. (Angulo P, Kleiner D E, Dam-Larsen S, Adams L A, Bjornsson E S, Charatcharoenwitthaya P, et al. Liver fibrosis, but no other histologic features, is associated with long-term outcomes of patients with nonalcoholic fatty liver disease. Gastroenterology. 2015; 149:389-397.e310.) Important clues to mechanisms and therapeutic targets can emerge from human genetic studies. Allelic variation at rs641738 C>T is a common polymorphism that genome-wide association studies and other genetic studies have shown is associated with lower expression of MBOAT7 and all stages of MASH, including liver fibrosis. (Busca C, Arias P, Sánchez-Conde M, Rico M, Montejano R, Martin-Carbonero L, et al. Genetic variants associated with steatohepatitis and liver fibrosis in HIV-infected patients with NAFLD. Front Pharmacol. 2022; 13:905126; Dongiovanni P, M Anstee Q, Valenti L. Genetic predisposition in NAFLD and NASH: Impact on severity of liver disease and response to treatment. Curr Pharmaceut Des. 2013; 19:5219-38; Krawczyk M, Rau M, Schattenberg J M, Bantel H, Pathil A, Demir M, et al. Combined effects of the PNPLA3 rs738409, TM6SF2 rs58542926, and MBOAT7 rs641738 variants on NAFLD severity: A multicenter biopsy-based study. J Lipid Res. 2017; 58:247-55; Luukkonen P K, Zhou Y, Hyötyläinen T, Leivonen M, Arola J, Orho-Melander M, et al. The MBOAT7 variant rs641738 alters hepatic phosphatidylinositols and increases severity of nonalcoholic fatty liver disease in humans. J Hepatol. 2016; 65:1263-5; Mancina R M, Dongiovanni P, Petta S, Pingitore P, Meroni M, Rametta R, et al. The MBOAT7-TMC4 variant rs641738 increases risk of nonalcoholic fatty liver disease in individuals of European descent. Gastroenterology. 2016; 150:1219-230. e1216; Meroni M, Longo M, Fracanzani A L, Dongiovanni P. MBOAT7 down-regulation by genetic and environmental factors predisposes to MAFLD. EBioMedicine. 2020; 57:102866; Pazoki R, Vujkovic M, Elliott J, Evangelou E, Gill D, Ghanbari M, et al. Genetic analysis in European ancestry individuals identifies 517 loci associated with liver enzymes. Nat Commun. 2021; 12:2579; Raja A M, Ciociola E, Ahmad I N, Dar F S, Naqvi S M S, Moaeen-Ud-Din M, et al. Genetic susceptibility to chronic liver disease in individuals from Pakistan. Int J Mol Sci.; Teo K, Abeysekera K W M, Adams L, Aigner E, Anstee Q M, Banales J M, et al. rs641738C>T near MBOAT7 is associated with liver fat, ALT and fibrosis in NAFLD: A meta-analysis. J Hepatol. 2021; 74:20-30; Thangapandi V R, Knittelfelder O, Brosch M, Patsenker E, Vvedenskaya O, Buch S, et al. Loss of hepatic Mboat7 leads to liver fibrosis. Gut. 2021; 70:940-50; Xu X, Xu H, Liu X, Zhang S, Cao Z, Qiu L, et al. MBOAT7 rs641738 (C>T) is associated with NAFLD progression in men and decreased ASCVD risk in elder Chinese population. Front Endocrinol (Lausanne). 2023; 14:1199429; Chen Y, Du X, Kuppa A, Feitosa M F, Bielak L F, O'Connell J R, et al. Genome-wide association meta-analysis identifies 17 loci associated with nonalcoholic fatty liver disease. Nat Genet. 2023; 55:1640-50) MBOAT7 (membrane-bound O-acyltransferase domain containing 7; aka lysophosphatidylinositol acyltransferase 1 [LPIAT1]) is an enzyme that carries out acyl chain remodeling, primarily involving arachidonic acid and phosphatidylinositol (PI). (Anderson K E, Kielkowska A, Durrant T N, Juvin V, Clark J, Stephens L R, et al. Lysophosphatidylinositol-acyltransferase-1 (LPIAT1) is required to maintain physiological levels of PtdIns and PtdInsP(2) in the mouse. PLoS One. 2013; 8:e58425; Lee H C, Inoue T, Imae R, Kono N, Shirae S, Matsuda S, et al. Caenorhabditis elegans mboa-7, a member of the MBOAT family, is required for selective incorporation of polyunsaturated fatty acids into phosphatidylinositol. Mol Biol Cell. 2008; 19: 1174-84)

Major gaps remain in the understanding of how MBOAT7 loss-of-function (LoF) increases the risk of MASH fibrosis. In particular, MBOAT7-LoF exacerbates hepatosteatosis, which in turn may contribute to liver fibrosis. (Krawczyk M, Rau M, Schattenberg J M, Bantel H, Pathil A, Demir M, et al. Combined effects of the PNPLA3 rs738409, TM6SF2 rs58542926, and MBOAT7 rs641738 variants on NAFLD severity: A multicenter biopsy-based study. J Lipid Res. 2017; 58:247-55; Luukkonen P K, Zhou Y, Hyötyläinen T, Leivonen M, Arola J, Orho-Melander M, et al. The MBOAT7 variant rs641738 alters hepatic phosphatidylinositols and increases severity of nonalcoholic fatty liver disease in humans. J Hepatol. 2016; 65: 1263-5; Mancina R M, Dongiovanni P, Petta S, Pingitore P, Meroni M, Rametta R, et al. The MBOAT7-TMC4 variant rs641738 increases risk of nonalcoholic fatty liver disease in individuals of European descent. Gastroenterology. 2016; 150:1219-230. e1216; Meroni M, Longo M, Fracanzani A L, Dongiovanni P. MBOAT7 down-regulation by genetic and environmental factors predisposes to MAFLD. EBioMedicine. 2020; 57:102866; Teo K, Abeysekera K W M, Adams L, Aigner E, Anstee Q M, Banales J M, et al. rs641738C>T near MBOAT7 is associated with liver fat, ALT and fibrosis in NAFLD: A meta-analysis. J Hepatol. 2021; 74:20-30; Thangapandi V R, Knittelfelder O, Brosch M, Patsenker E, Vvedenskaya O, Buch S, et al. Loss of hepatic Mboat7 leads to liver fibrosis. Gut. 2021; 70:940-50; Buch S, Stickel F, Trepo E, Way M, Herrmann A, Nischalke H D, et al. A genome-wide association study confirms PNPLA3 and identifies TM6SF2 and MBOAT7 as risk loci for alcohol-related cirrhosis. Nat Genet. 2015; 47:1443-8; Helsley R N, Varadharajan V, Brown A L, Gromovsky A D, Schugar R C, Ramachandiran I, et al. Obesity-linked suppression of membrane-bound O-acyltransferase 7 (MBOAT7) drives nonalcoholic fatty liver disease. eLife. 2019; 8:e49882; Tanaka Y, Shimanaka Y, Caddeo A, Kubo T, Mao Y, Kubota T, et al. LPIAT1/MBOAT7 depletion increases triglyceride synthesis fueled by high phosphatidylinositol turnover. Gut. 2021; 70: 180-93; Varadharajan V, Massey W J, Brown J M. Membrane-bound O-acyltransferase 7 (MBOAT7)-driven phosphatidylinositol remodeling in advanced liver disease. J Lipid Res. 2022; 63:100234; Xia M, Chandrasekaran P, Rong S, Fu X, Mitsche M A. Hepatic deletion of Mboat7 (LPIAT1) causes activation of SREBP-1c and fatty liver. J Lipid Res. 2021; 62:100031). However, the possibility that hepatocyte MBOAT7 LoF contributes to liver fibrosis by a distinct, direct mechanism remains unexplored. Germline deletion of hepatocyte Mboat7 in a mouse MASH model led to increased liver lysoPI and fibrosis, but the precise mechanism was not defined. (Thangapandi V R, Knittelfelder O, Brosch M, Patsenker E, Vvedenskaya O, Buch S, et al. Loss of hepatic Mboat7 leads to liver fibrosis. Gut. 2021; 70:940-50). In another study, Mboat7 antisense oligonucleotides given at the start of high-fat diet feeding increased insulin resistance, hepatosteatosis, and fibrosis-associated mRNA, (Helsley R N, Varadharajan V, Brown A L, Gromovsky A D, Schugar R C, Ramachandiran I, et al. Obesity-linked suppression of membrane-bound O-acyltransferase 7 (MBOAT7) drives nonalcoholic fatty liver disease. eLife. 2019; 8:e49882) but whether the mechanism was independent of hepatosteatosis could not be determined. Similar results were found when liver MBOAT7 was deleted before the initiation of high-fat diet feeding. (Tanaka Y, Shimanaka Y, Caddeo A, Kubo T, Mao Y, Kubota T, et al. LPIAT1/MBOAT7 depletion increases triglyceride synthesis fueled by high phosphatidylinositol turnover. Gut. 2021; 70: 180-93.) In another mouse MASH study, supraphysiologic overexpression of MBOAT7 showed modest beneficial effects on liver triglyceride content and plasma ALT and AST, but liver fibrosis was not improved (Sharpe M C, Pyles K D, Hallcox T, Kamm D R, Piechowski M, Fisk B, et al. Enhancing hepatic MBOAT7 expression in mice with nonalcoholic steatohepatitis. Gastro Hep Adv. 2023; 2:558-72), further questioning the relationship between MBOAT7 and MASH fibrosis.

This question was addressed by designing MASH experiments that would focus specifically on MASH fibrosis and, importantly, by seeking a plausible, direct mechanistic link between MBOAT7 LoF and liver fibrosis. Described herein is evidence that hepatocyte MBOAT7 directly affects liver fibrosis in experimental mice in a steatosis-independent manner. Restoration of hepatocyte MBOAT7 in mice with diet-induced MASLD to a level similar to that in chow-fed mice lowered liver fibrosis without affecting hepatosteatosis. Conversely, when hepatocyte-MBOAT7 was silenced after the development of MASLD, liver fibrosis but not hepatosteatosis was exacerbated. Described herein is evidence supporting a new theory that could link MBOAT7 LoF specifically to MASH fibrosis, namely, lipid changes in hepatocytes caused by MBOAT7 LoF promote a cholesterol-induced profibrotic pathway in MASH involving upregulation of the gene regulator TAZ (WWTR1). (Wang X, Zheng Z, Caviglia J M, Corey K E, Herfel T M, Cai B, et al. Hepatocyte TAZ/WWTR1 promotes inflammation and fibrosis in nonalcoholic steatohepatitis. Cell Metab. 2016; 24: 848-62; Wang X, Sommerfeld M R, Jahn-Hofmann K, Cai B, Filliol A, Remotti H E, et al. A therapeutic silencing RNA targeting Hepatocyte TAZ prevents and reverses fibrosis in nonalcoholic steatohepatitis in mice. Hepatol Commun. 2019; 3:1221-34; Wang X, Cai B, Yang X, Sonubi O O, Zheng Z, Ramakrishnan R, et al. Cholesterol stabilizes TAZ in hepatocytes to promote experimental non-alcoholic steatohepatitis. Cell Metab. 2020; 31: 969-986.e967; Wang X, Moore M P, Shi H, Miyata Y, Donnelly S K, Radiloff D R, et al. Hepatocyte-targeted siTAZ therapy lowers liver fibrosis in NASH diet-fed chimeric mice with hepatocyte-humanized livers. Mol Ther Methods Clin Dev. 2023; 31:101165). This theory is supported with analyses of hepatocytes, including primary human hepatocytes, the above mouse models, and human MASH liver specimens from individuals with the hypomorphic MBOAT7 rs641738 C>T risk variant. In summary, described herein is evidence for a direct, mechanistically plausible link between MBOAT7 LoF and MASH fibrosis and provide new insights into an established genetic locus for MASH. Moreover, given the druggability of the TAZ pathway for MASH fibrosis (Wang X, Sommerfeld M R, Jahn-Hofmann K, Cai B, Filliol A, Remotti H E, et al. A therapeutic silencing RNA targeting Hepatocyte TAZ prevents and reverses fibrosis in nonalcoholic steatohepatitis in mice. Hepatol Commun. 2019; 3:1221-34; Wang X, Moore M P, Shi H, Miyata Y, Donnelly S K, Radiloff D R, et al. Hepatocyte-targeted siTAZ therapy lowers liver fibrosis in NASH diet-fed chimeric mice with hepatocyte-humanized livers. Mol Ther Methods Clin Dev. 2023; 31:101165), this study suggests a personalized medicine approach to lower the risk of MASH fibrosis in individuals harboring genetic variants that lower MBOAT7.

### Methods

Animal studies Male wild-type C57BL/6J mice (#000664, 10-11 wk/old) from Jackson Laboratory were allowed to adapt in the CUIMC ICM for 1 week before random assignment to experimental cohorts. The mice were fed a fructosepalmitate diet containing 1.25% cholesterol (FPC [fructose-palmitate-cholesterol diet]; Teklad, TD.160785) and sugar water (23.1 g/L fructose and 18.9 g/L glucose) for the times and under the treatments indicated in the figures. Mboat7^{fl/fl} mice, generated as described, (Xia M, Chandrasekaran P, Rong S, Fu X, Mitsche M A. Hepatic deletion of Mboat7 (LPIAT1) causes activation of SREBP-1c and fatty liver. J Lipid Res. 2021; 62:100031) were purchased from the International Mouse Phenotyping Consortium (www.mousephenotype.org/data/genes/MGI:1924832). Animals were housed in standard cages at 22° C. in a 12-12-h light-dark cycle in a barrier facility. All experiments were approved by the Institutional Animal Care and Use Committee at Columbia.

### Human Samples

The relationship between liver nuclear (active) TAZ and rs641738-T was examined in 103 patients with MASH who participated in the FLINT trial (CTA #: NCT01265498). Liver sections were obtained from a subset of these patients with MASH at the baseline visit (FIG. 20, http://links.lww.com/HEP/I469), whose characteristics have previously been published. (Neuschwander-Tetri B A, Loomba R, Sanyal A J, Lavine J E, Van Natta M L, Abdelmalek M F, et al. Farnesoid X nuclear receptor ligand obeticholic acid for non-cirrhotic, non-alcoholic steatohepatitis (FLINT): A multicentre, randomised, placebo-controlled trial. Lancet. 2015; 385:956-65) Diagnosis of MASH was determined from blinded centrally reviewed liver biopsy slides by the Pathology Committee. All participants provided consent to the use of these specimens, and the protocol was reviewed and approved by local IRBs and all CRN Steering Committee members. The relationship between liver IHH expression and rs641738-T was examined in 123 obese individuals who underwent percutaneous liver biopsy during bariatric surgery (Baselli G A, Dongiovanni P, Rametta R, Meroni M, Pelusi S, Maggioni M, et al. Liver transcriptomics highlights interleukin-32 as novel NAFLD-related cytokine and candidate biomarker. Gut. 2020; 69:1855-66) (FIG. 21, links.lww.com/HEP/1469) and for whom material for high-quality RNA extraction was available and the variant could be genotyped. Transcriptomic analysis of the livers, which is publicly available for some genes, was conducted as described. (Cherubini A, Ostadreza M, Jamialahmadi O, Pelusi S, Rrapaj E, Casirati E, et al. Interaction between estrogen receptor-α and PNPLA3 p. I148M variant drives fatty liver disease susceptibility in women. Nat Med. 2023; 29:2643-55) Patients gave informed consent, and protocols were approved by the Ethical Committee of the Fondazione IRCCS Ca' Granda Milan and conducted according to the World's Medical Association Declaration. Patient records were pseudo-anonymized and deidentified.

### Cell Culture and Cell Treatment

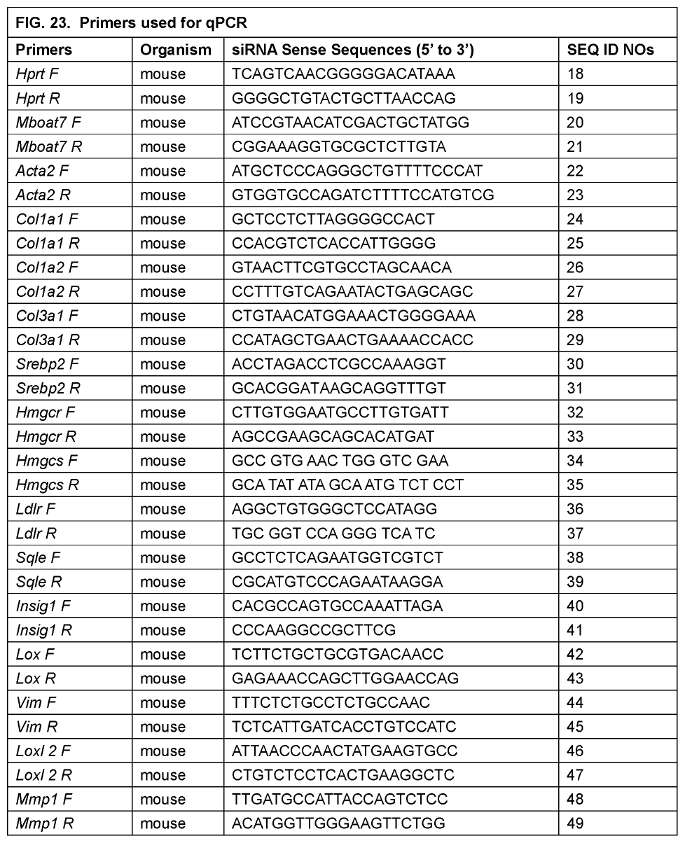

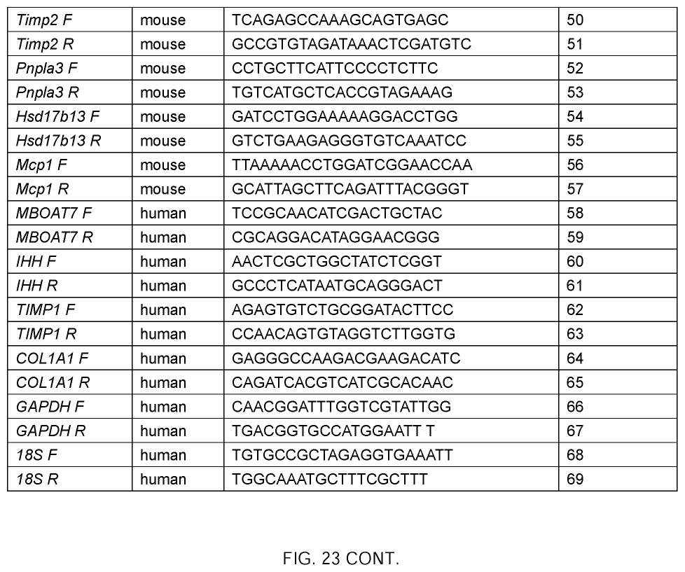

AML12 mouse hepatocytes were purchased from ATCC (CRL-2254) and cultured in DMEM/F12 medium (Life Technologies, #11320) with 10% (vol/vol) heatinactivated FBS (Gibco, #16140-071) and 1× penicillin-streptomycin solution (Corning, #30-002-Cl). Primary mouse hepatocytes were isolated as described (Wang X, Cai B, Yang X, Sonubi O O, Zheng Z, Ramakrishnan R, et al. Cholesterol stabilizes TAZ in hepatocytes to promote experimental non-alcoholic steatohepatitis. Cell Metab. 2020; 31: 969-986.e967; Ozcan L, Wong C C, Li G, Xu T, Pajvani U, Park S K, et al. Calcium signaling through CaMKII regulates hepatic glucose production in fasting and obesity. Cell Metab. 2012; 15:739-51) from 10-week-old wild-type C57BL/6J mice or Mboat7^{fl/fl} mice 7 days after injection with AAV8-TBGCre (to delete hepatocyte MBOAT7) or AAV8-TBG-GFP control virus (1.5×1011 genome copies/mouse). All cells were grown at 37° C. in a 5% CO2 incubator. Primary human hepatocytes were obtained from the Human Hepatocyte Isolation Distribution program from the Clinical Biospecimen Repository and Processing Core through the Pittsburgh Liver Research Center. The siRNA sequences are listed in FIG. 22. The primer sequences are listed in FIG. 23.

### Assaying the Accessible Plasma Membrane Cholesterol Pool in AML12 Hepatocytes

Accessible cholesterol in the plasma membrane (PM), but not inaccessible cholesterol, interacts with a nonlytic peptide encompassing domain 4 of anthrolysin O called ALOD4. (Johnson K A, Radhakrishnan A. The use of anthrolysin O and ostreolysin A to study cholesterol in cell membranes. Methods Enzymol. 2021; 649:543-66) The binding of His6-tagged ALOD4 (“His6” disclosed as SEQ ID NO: 70) to the surface of cells can thus be used to monitor the accessible cholesterol pool by immunofluorescence microscopy or by western blot, as previously described. (Ferrari A, He C, Kennelly J P, Sandhu J, Xiao X, Chi X, et al. Aster proteins regulate the accessible cholesterol pool in the plasma membrane. Mol Cell Biol. 2020; 40; He C, Hu X, Weston T A, Jung R S, Sandhu J, Huang S, et al. Macrophages release plasma membrane-derived particles rich in accessible cholesterol. Proc Natl Acad Sci USA. 2018; 115: E8499-508; Xiao X, Kennelly J P, Ferrari A, Clifford B L, Whang E, Gao Y, et al. Hepatic nonvesicular cholesterol transport is critical for systemic lipid homeostasis. Nat Metab. 2023; 5:165-81). Briefly, control or Mboat7-silenced AML12 cells were depleted of sterols by incubating in a medium containing 1% LPDS, simvastatin (5 μM), and mevalonate (10 μM) for 16 hours at 37° C. The cells were then refreshed with the same media in the absence or presence of mβCD-cholesterol, followed by washing 3 times with HBSS (Ca2+/Mg2+) containing 0.2% BSA and then incubation with His6-tagged ALOD4 (20 μg/mL) (“His6” disclosed as SEQ ID NO: 70), as described. (Xiao X, Kennelly J P, Ferrari A, Clifford B L, Whang E, Gao Y, et al. Hepatic nonvesicular cholesterol transport is critical for systemic lipid homeostasis. Nat Metab. 2023; 5:165-81) For imaging, the cells were washed 3 times with HBSS (Ca2+/Mg2+) containing 0.2% BSA and then fixed with 3% paraformaldehyde for 15 minutes. After fixation, the cells were incubated overnight with an anti-His6 primary antibody (“His6” disclosed as SEQ ID NO: 70) (27E8, Cell Signaling, 2366S) and then with Alexa Fluor 488-labeled secondary antibodies for 1 hour at room temperature (goat anti-mouse IgG H&L, A 11001, Invitrogen). The cells were mounted with Prolong Diamond Antifade with DAPI (Invitrogen, P36962), and images were taken with a Leica TCS-SP8-SMD confocal microscope. For western blots, cells were lysed in RIPA before being probed with anti-His6 (“His6” disclosed as SEQ ID NO: 70) (27E8, Cell Signaling, 2366S) or anti-calnexin (Abcam ab10286) antibodies. A separate set of cholesterol depleted or repleted AML12 cells that were not incubated with ALOD4 were harvested for analysis of the cholesterol biosynthetic pathway by qPCR

### Statistical Analyses

Results are presented as means±SEM, and differences were considered statistically significant at p≤0.05. After passing normality testing using the Kolmogorov-Smirnov test, the data were analyzed using Student t test for 2 groups or one-way ANOVA with least-significant-difference post hoc analysis. The human liver-nuclear TAZ data were analyzed using one-way ANOVA. Statistically significant data were followed up with Tukey post hoc analyses. For the human TAZ data, a one-way ANCOVA was performed with sex, age, BMI, and fasting blood glucose as covariates for MBOAT7 rs641738, PNPLA3 rs738409, and HSD17B13 rs72613567. The human liver-IHH mRNA data were log-transformed, and one-way ANCOVA was performed with sex, age, blood glucose, LDL-cholesterol, and HDL-cholesterol as covariates for MBOAT7 rs641738 and PNPLA3 rs738409; and sex, age, and body mass index as covariates for HSD17B13 rs72613567. Sidak post hoc analyses were used for groups of 3 or more significant main effects. Analyses were performed using IBM SPSS Statistics software.

### Results

Hepatocyte MBOAT7 Restoration in Mice Suppresses the Progression to Early MASH Fibrosis without Affecting Hepatosteatosis

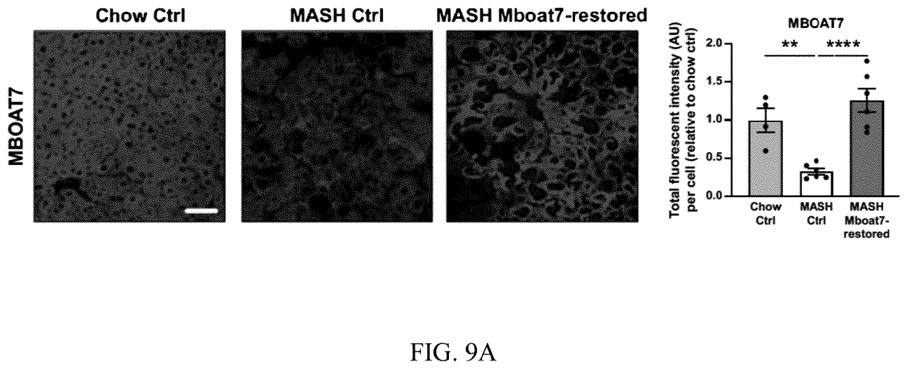

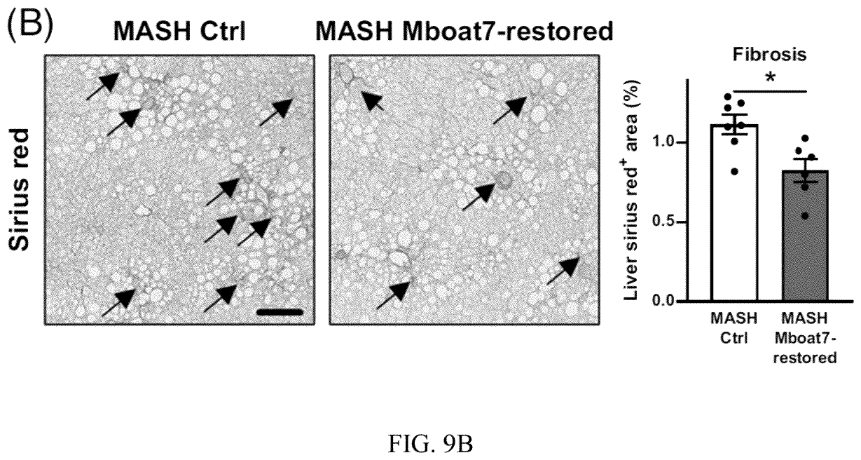

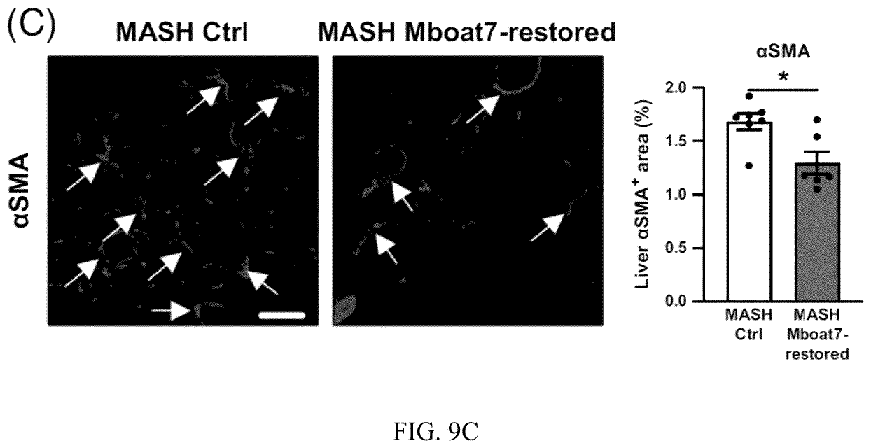

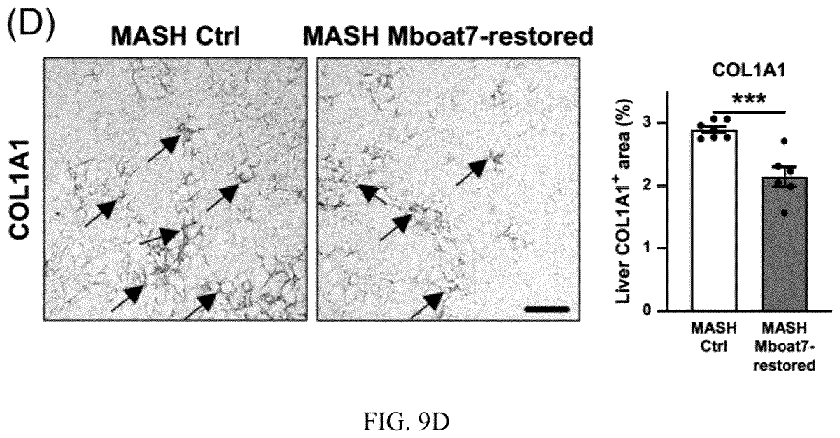

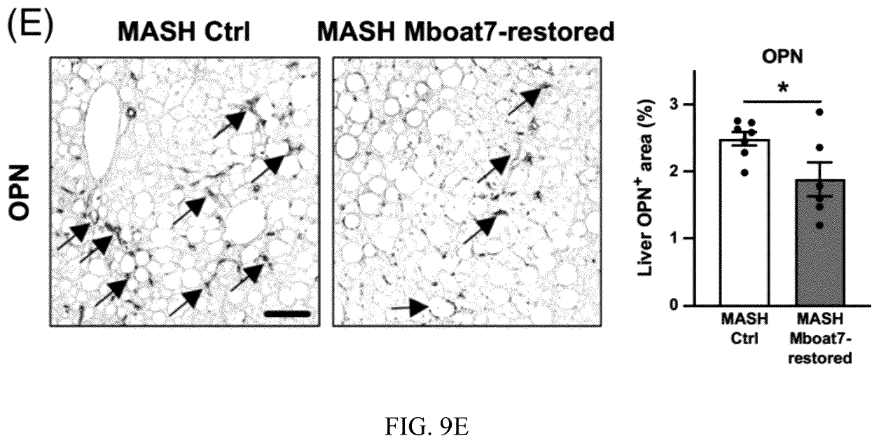

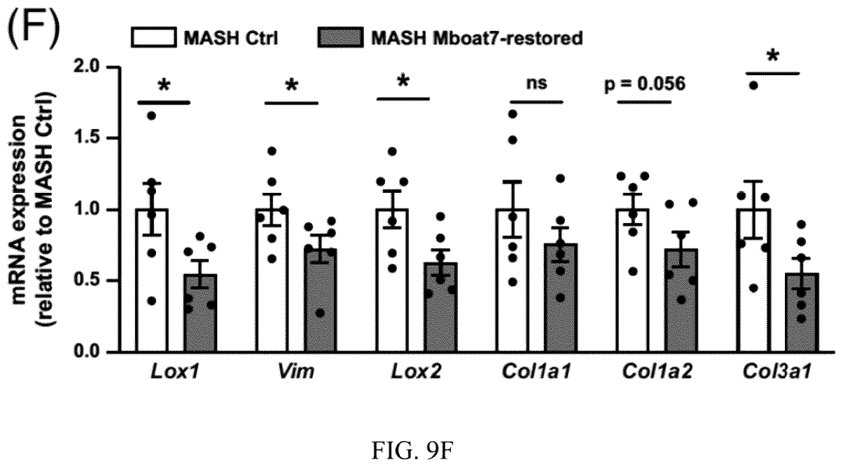

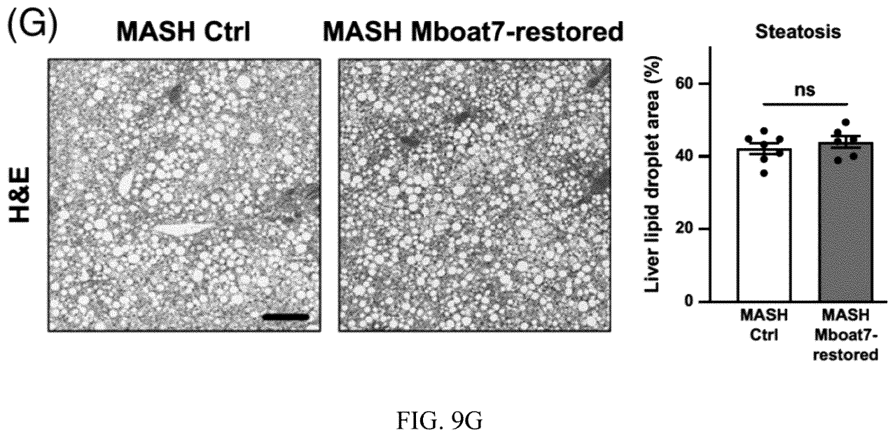

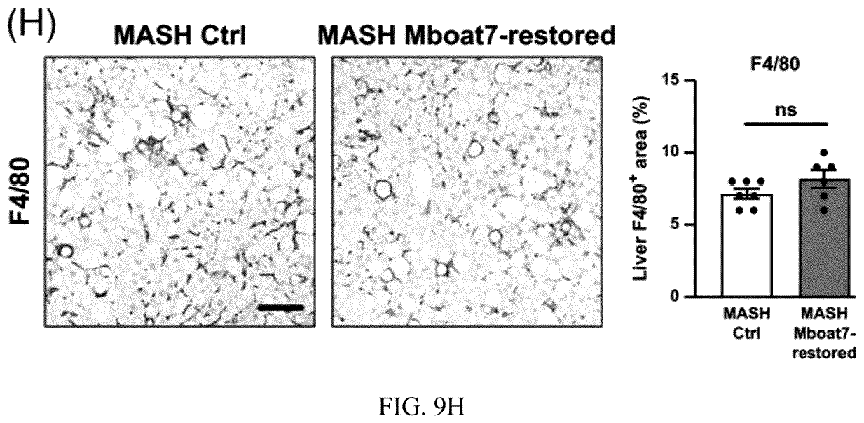

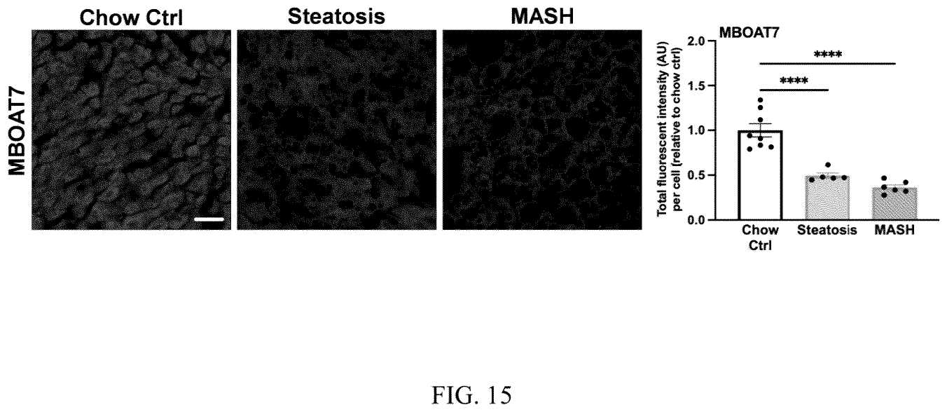

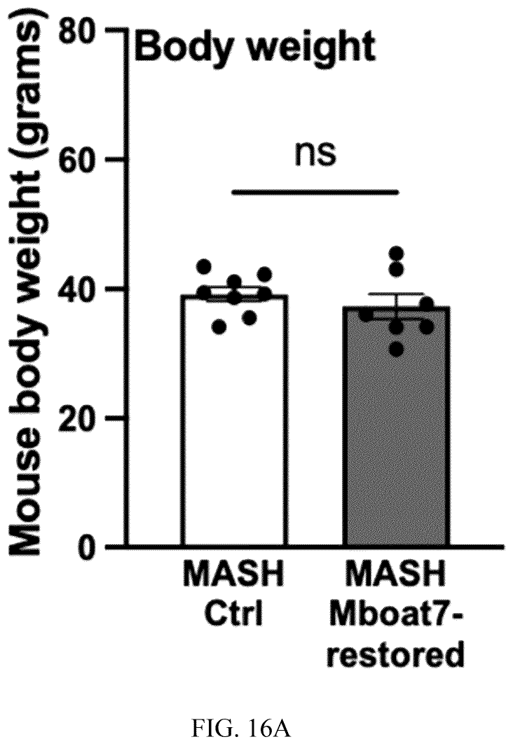

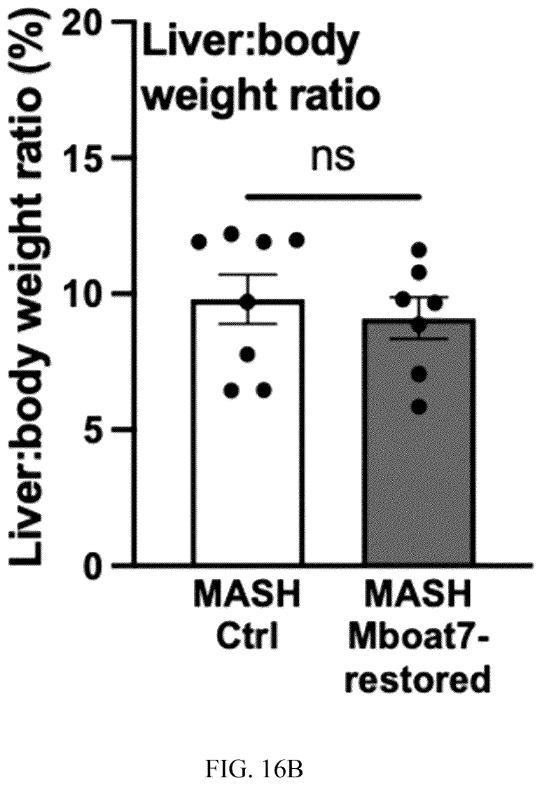

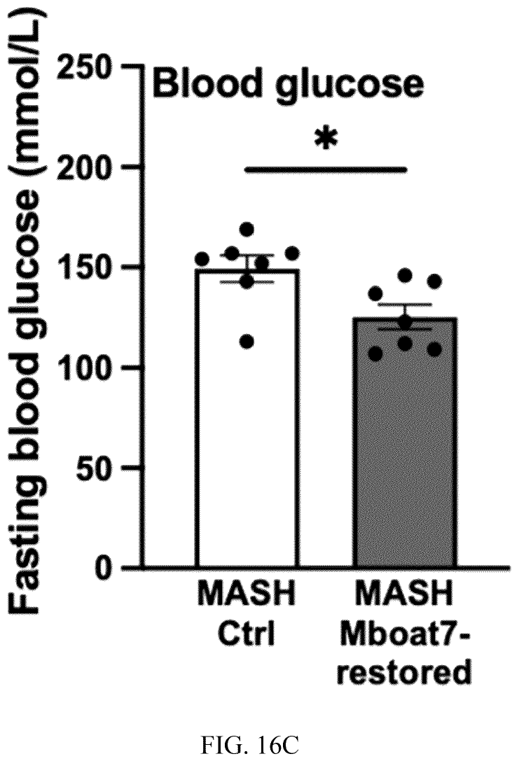

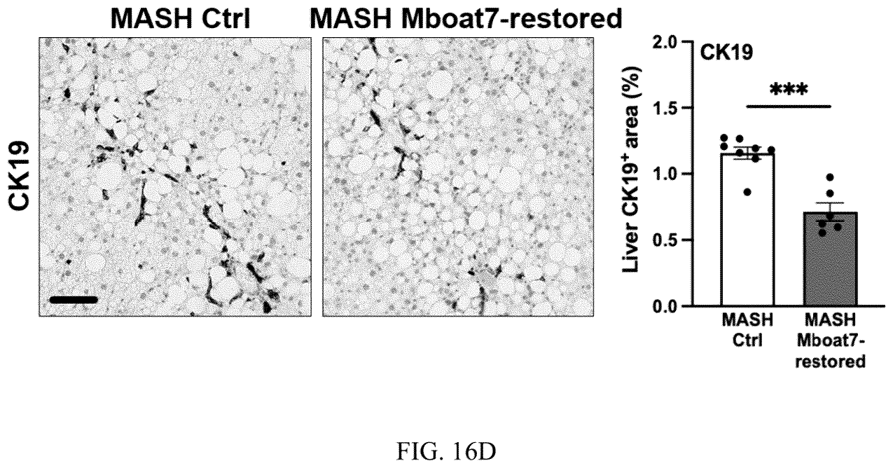

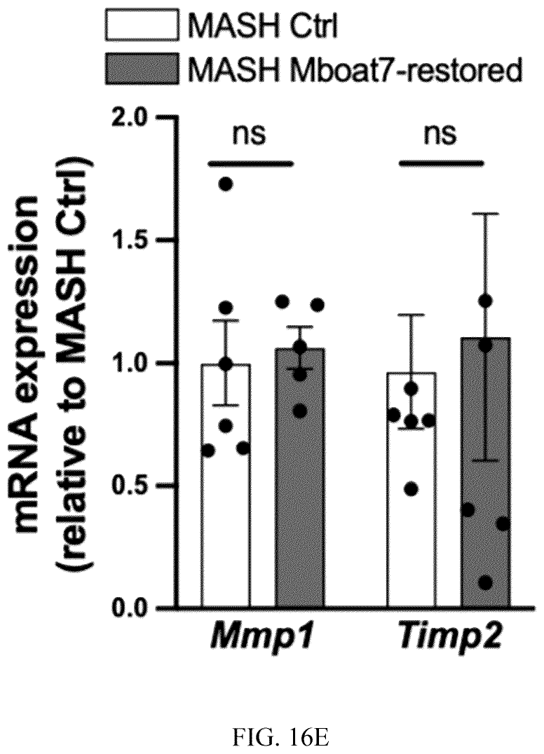

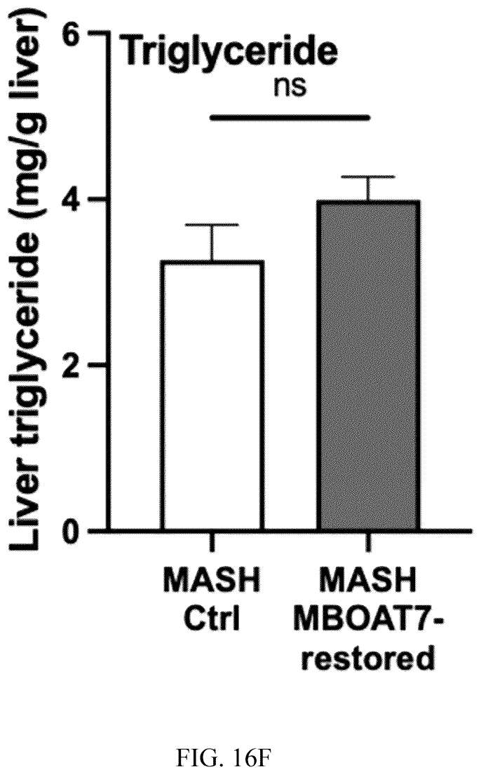

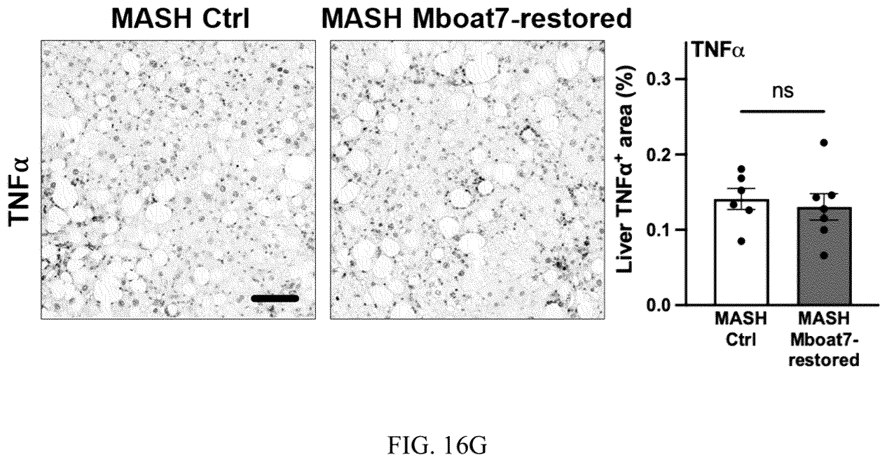

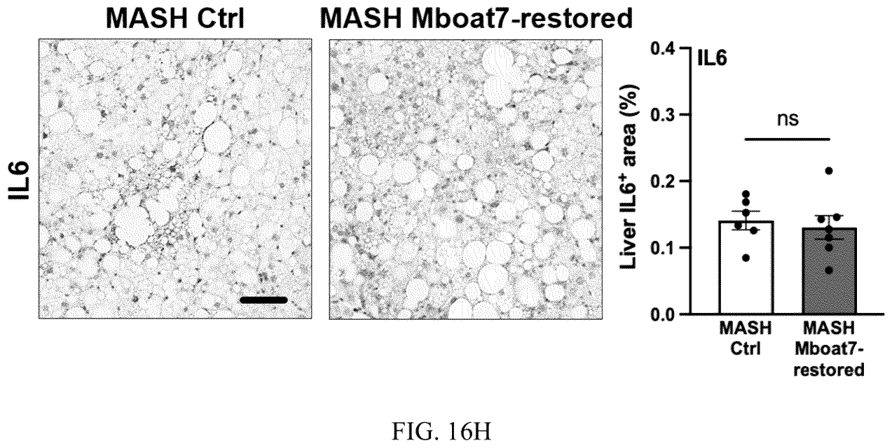

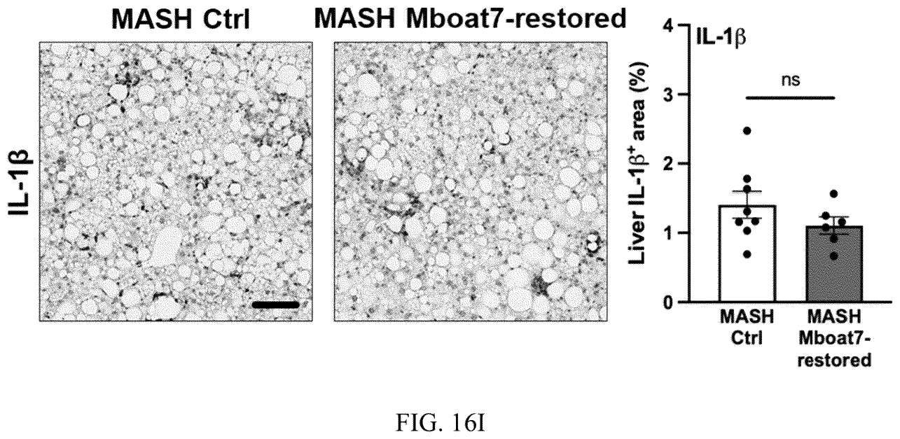

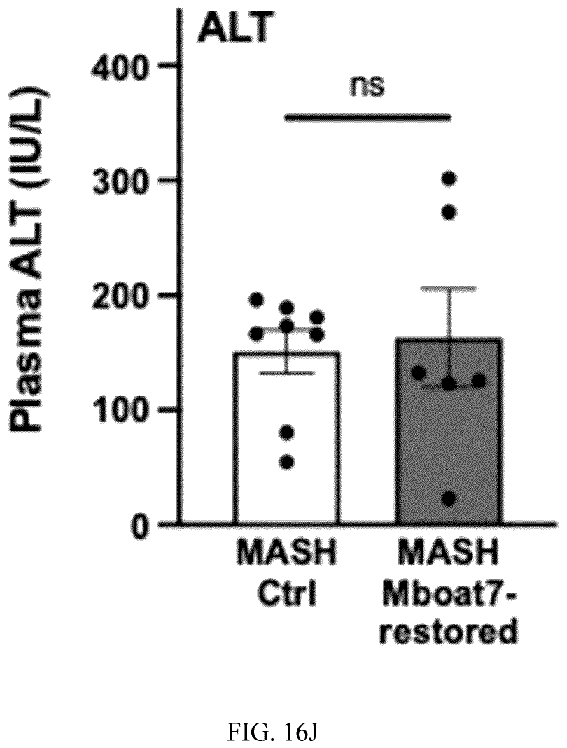

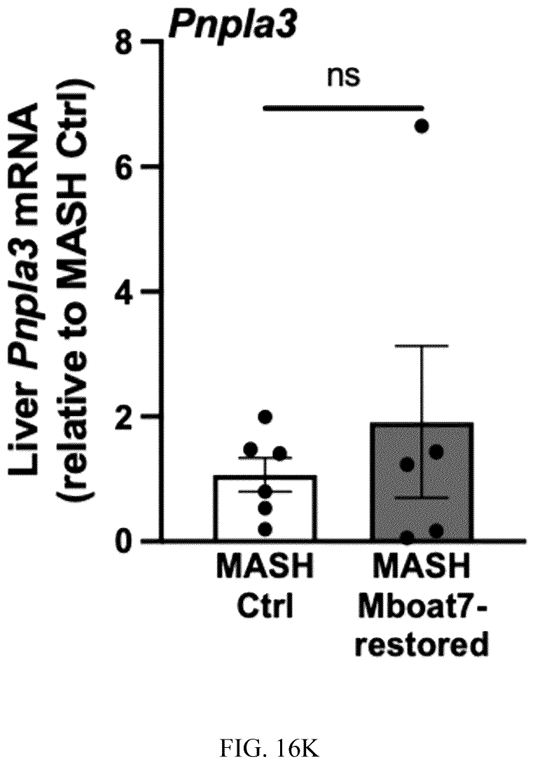

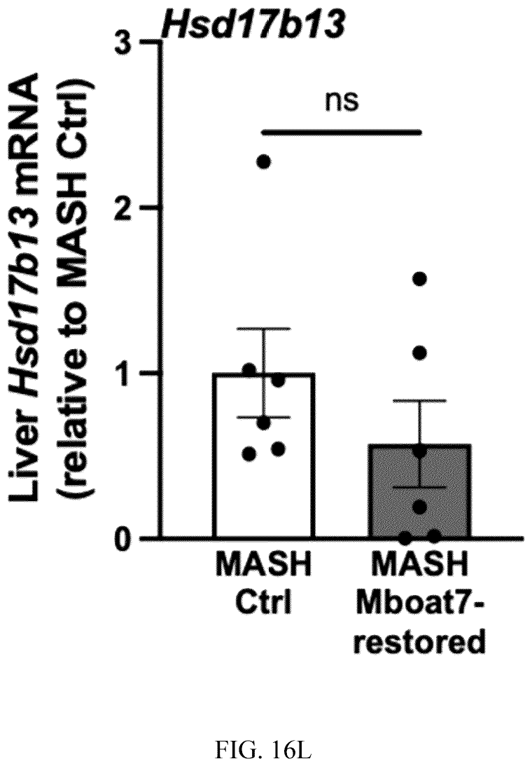

Liver MBOAT7 is decreased following the development of hepatosteatosis and early MASH (Helsley R N, Varadharajan V, Brown A L, Gromovsky A D, Schugar R C, Ramachandiran I, et al. Obesity-linked suppression of membrane-bound O-acyltransferase 7 (MBOAT7) drives nonalcoholic fatty liver disease. eLife. 2019; 8:e49882) in mice fed the human-relevant MASH inducing FPC diet (Wang X, Zheng Z, Caviglia J M, Corey K E, Herfel T M, Cai B, et al. Hepatocyte TAZ/WWTR1 promotes inflammation and fibrosis in nonalcoholic steatohepatitis. Cell Metab. 2016; 24: 848-62) for 8 or 16 weeks, respectively (FIG. 15). To determine if this decrease in MBOAT7 played a role in steatosis-to-MASH progression, 8-week FPC-fed mice were injected with AAV8-TBGMboat7 to restore MBOAT7 specifically in hepatocytes. Control mice received AAV8-TBG-GFP. The mice were then maintained on the FPC diet for an additional 8 weeks. The livers of the AAV8-TBGMboat7-treated mice expressed MBOAT7 to a level similar to the livers of chow-fed mice (FIG. 9A), and MBOAT7 restoration did not affect body weight or liver:body weight ratio (FIG. 21A, B), although there was a modest decrease in fasting blood glucose (FIG. 21C). Most importantly, fibrosis progression was decreased in the MBOAT7-restored mice based on decreases in picrosirius red, alpha-smooth muscle actin, collagen 1a1 (COL1A1), and osteopontin staining (FIGS. 9B-E) and by lower expression of HSC/fibrosis-related mRNAs (FIG. 9F). In addition, cytokeratin 19-positive area, a marker of bile ductular reaction in liver injury and fibrosis in MASH (Lee S J, Park J B, Kim K H, Lee W R, Kim J Y, An H J, et al. Immunohistochemical study for the origin of ductular reaction in chronic liver disease. Int J Clin Exp Pathol. 2014; 7:4076-85; Zhao L, Westerhoff M, Pai R K, Choi W T, Gao Z H, Hart J. Centrilobular ductular reaction correlates with fibrosis stage and fibrosis progression in non-alcoholic steatohepatitis. Mod Pathol. 2018; 31:150-9), was reduced in the AAV8-TBG-Mboat7-treated cohort (FIG. 16D). However, Mmp1 and Timp2 mRNAs, which encode extracellular matrix-degrading proteins, were similar in the 2 groups of mice (FIG. 16E). Similarly, hepatocyte-MBOAT7 restoration did not affect hepatosteatosis, liver inflammation (TNFα, IL6, IL-1β), plasma ALT, or the expression of Pnpla3 or Hsd17b13, which encode proteins associated with genetic susceptibility to MASH. (Dongiovanni P, Romeo S, Valenti L. Genetic factors in the pathogenesis of nonalcoholic fatty liver and steatohepatitis. Biomed Res Int. 2015; 2015:460190) (FIG. 9G, H and FIG. 16F-L). In summary, hepatocyte-MBOAT7 in mice with hepatosteatosis suppressed the progression to early MASH fibrosis.

Hepatocyte MBOAT7 Deletion in Mice Exacerbates MASH Fibrosis without Affecting Hepatosteatosis

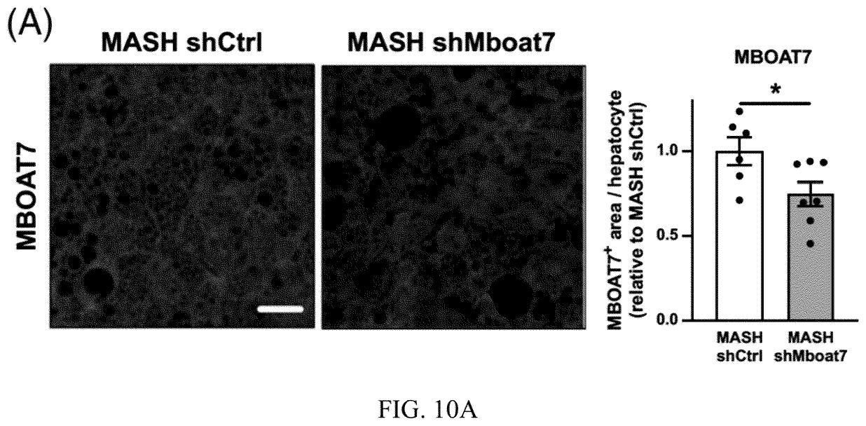

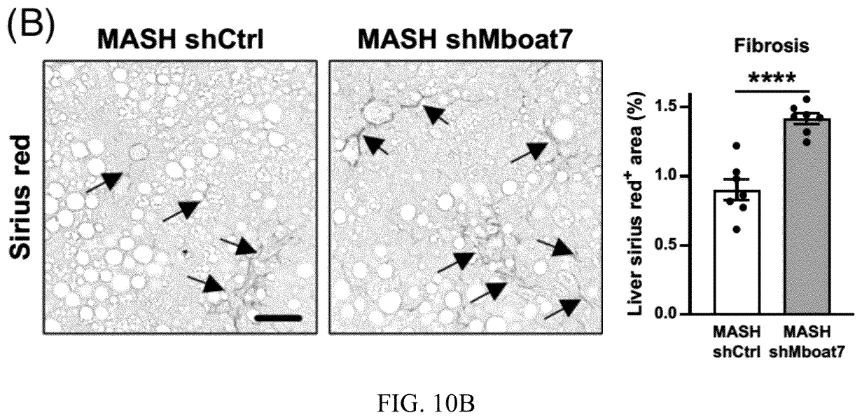

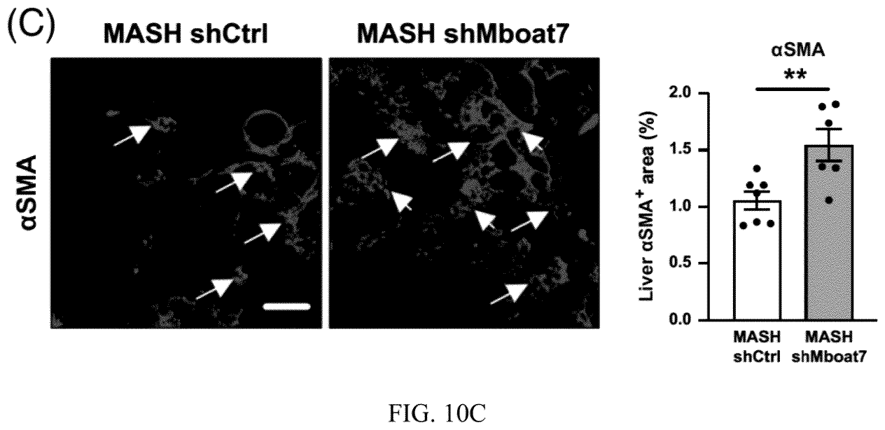

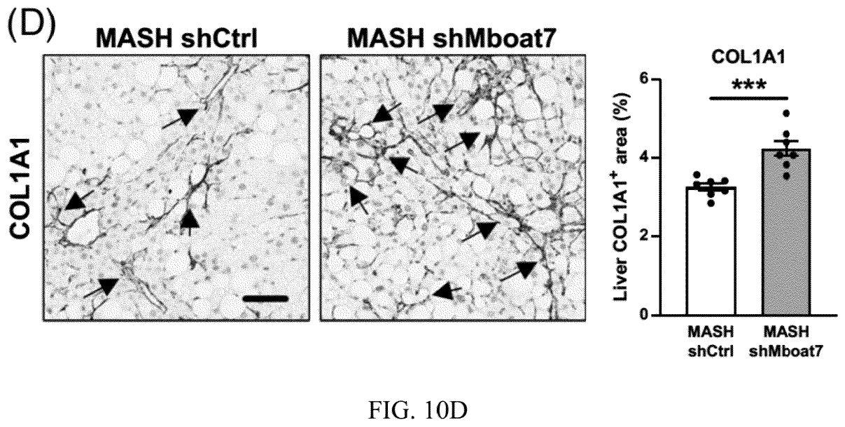

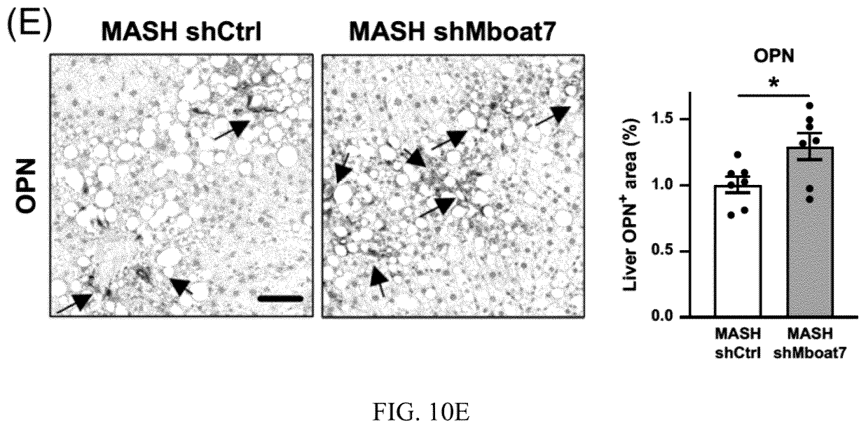

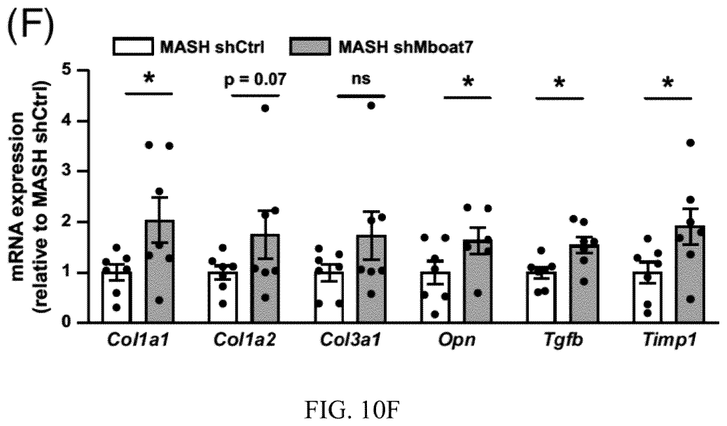

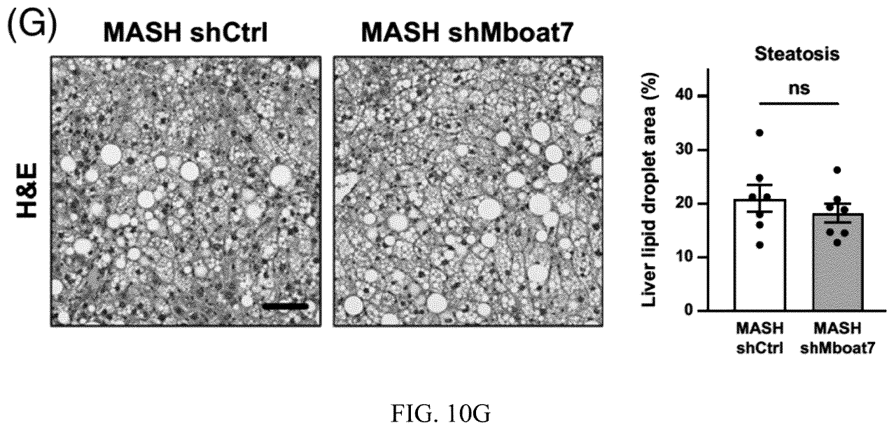

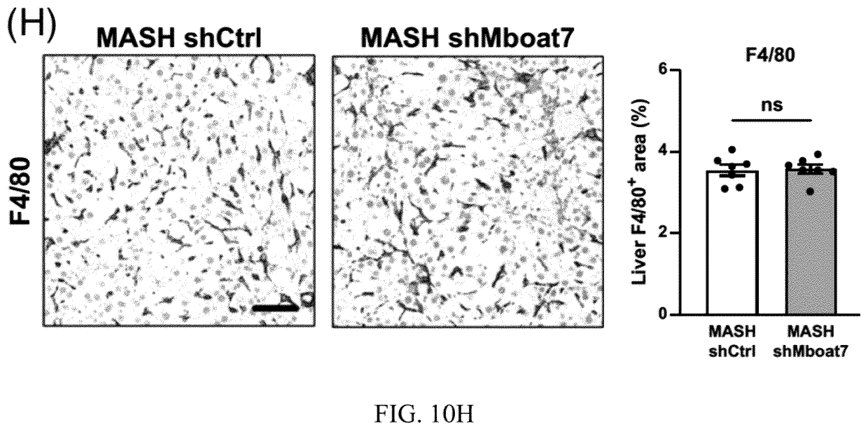

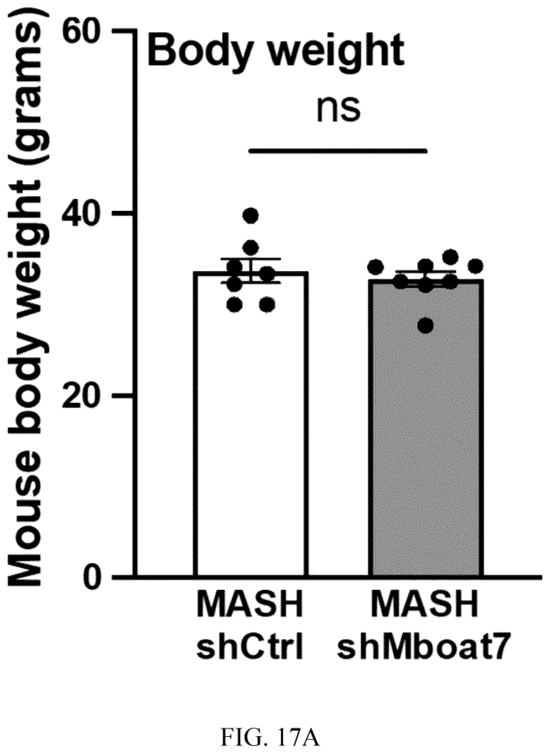

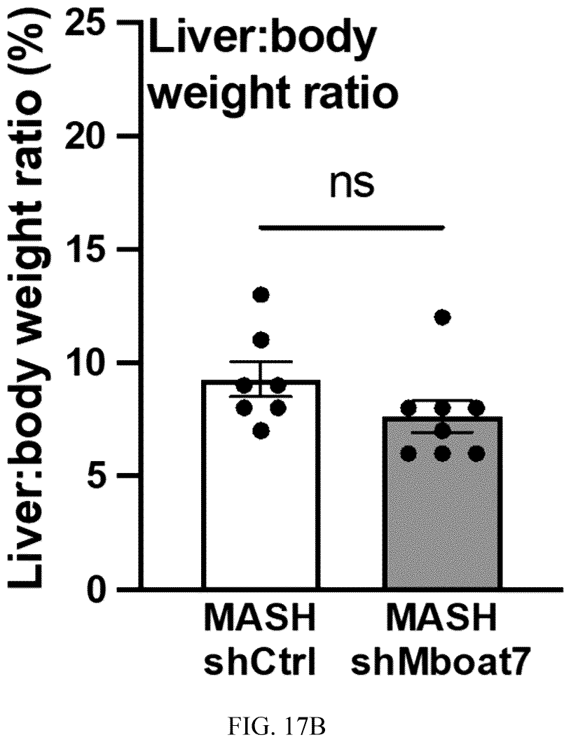

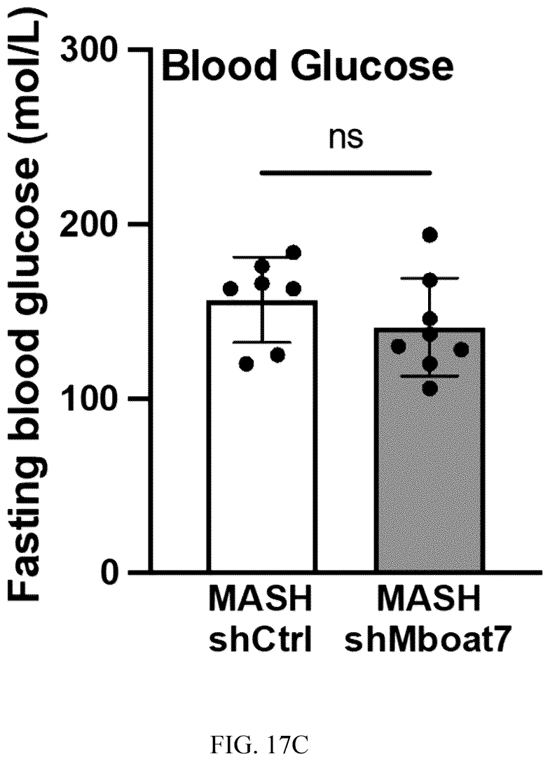

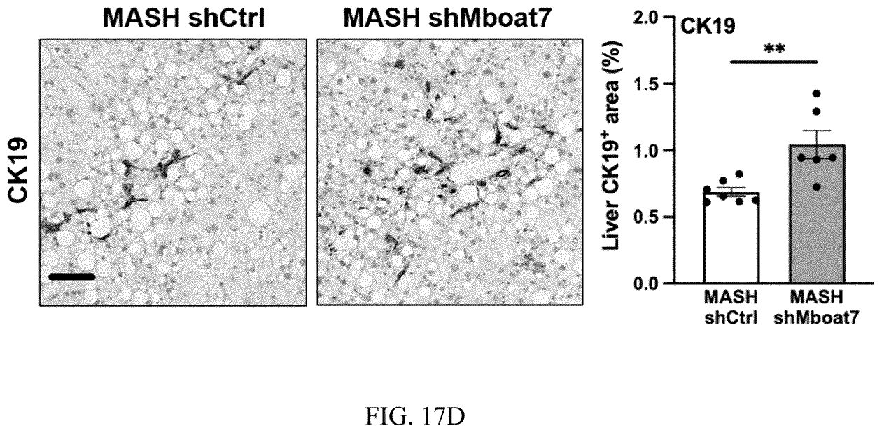

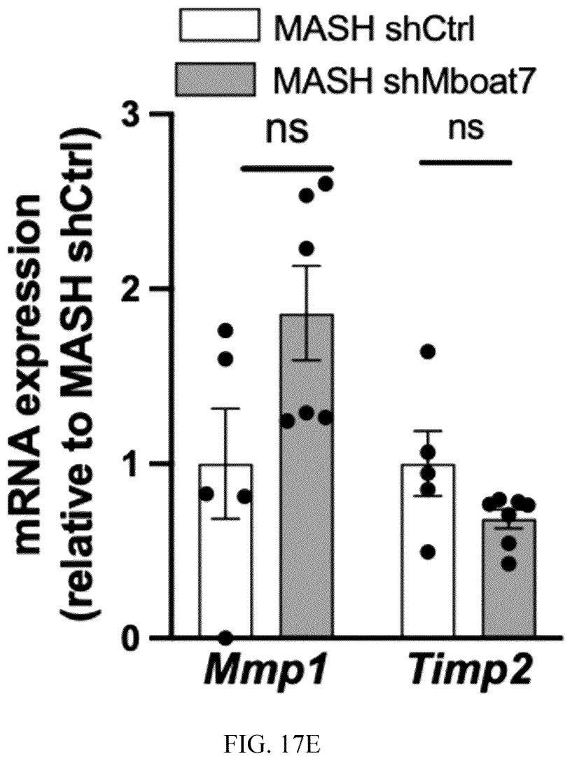

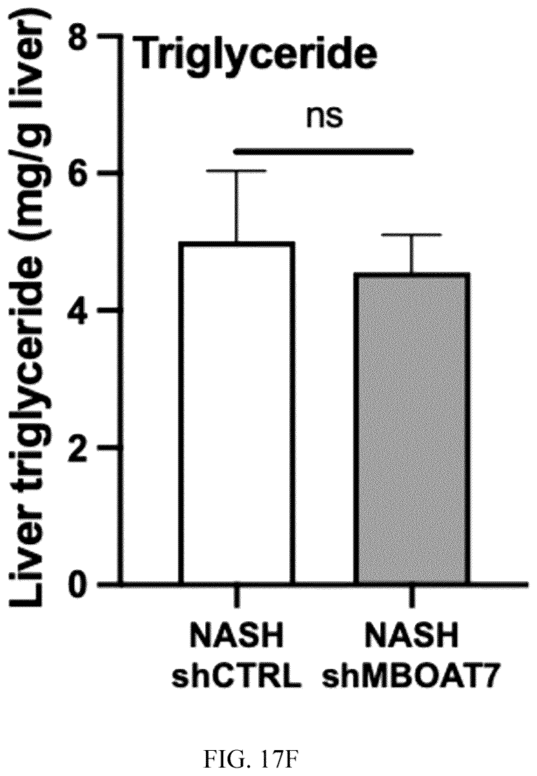

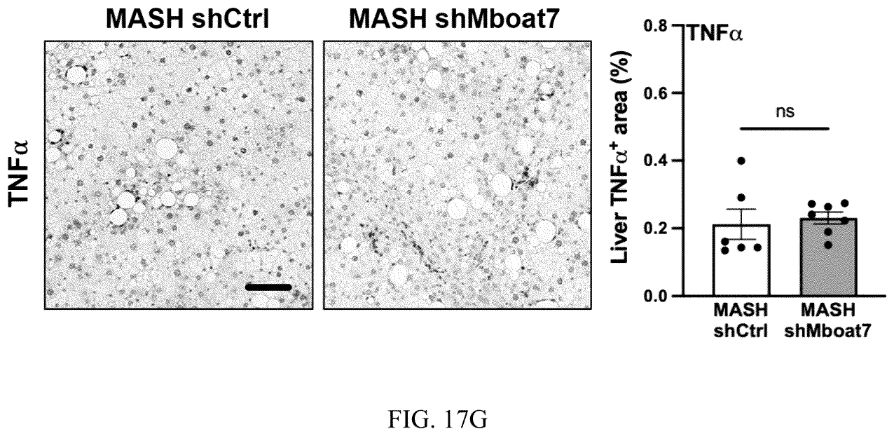

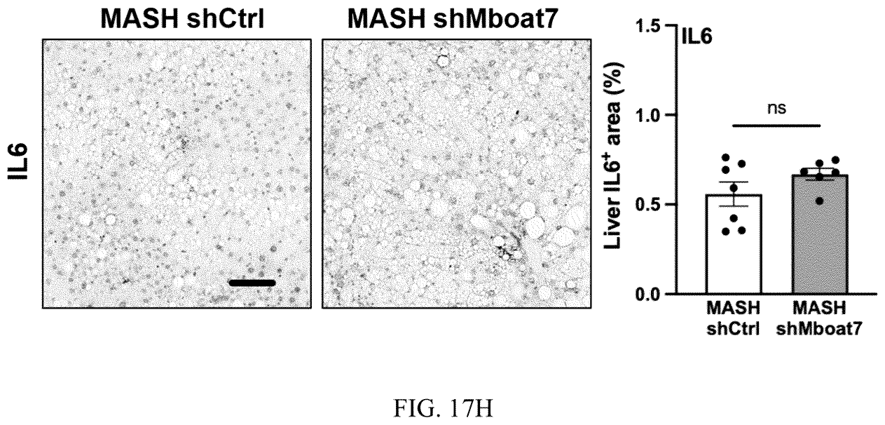

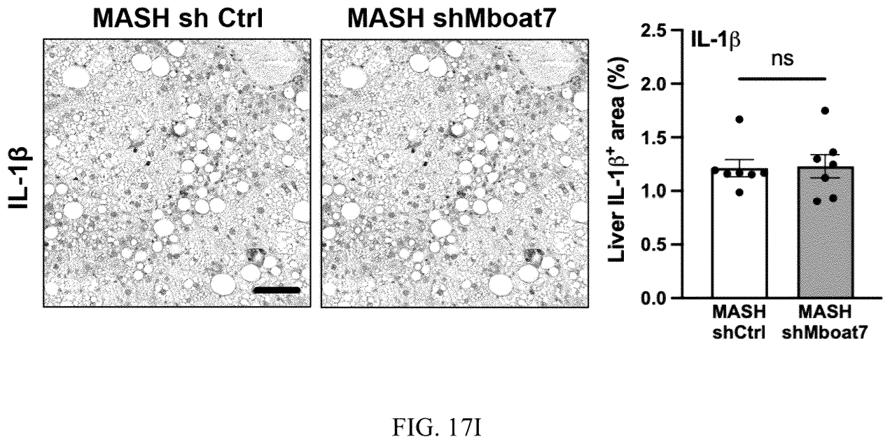

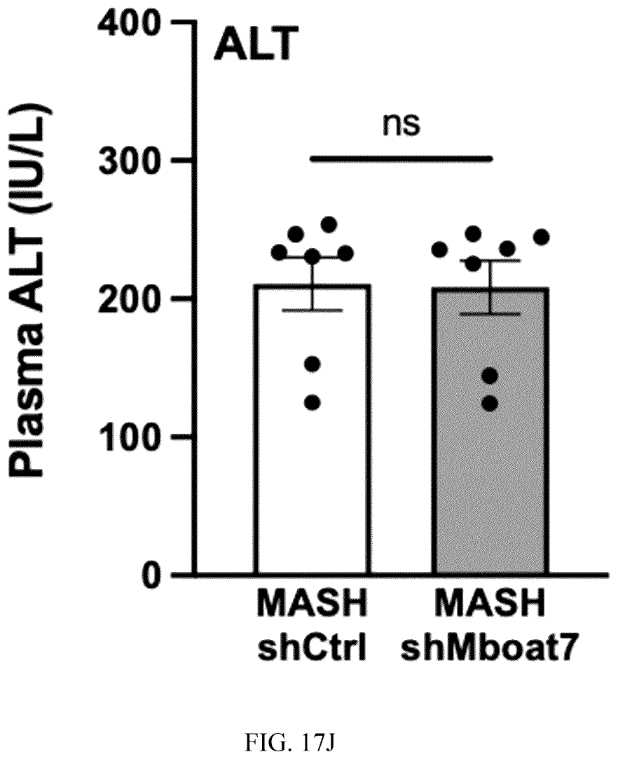

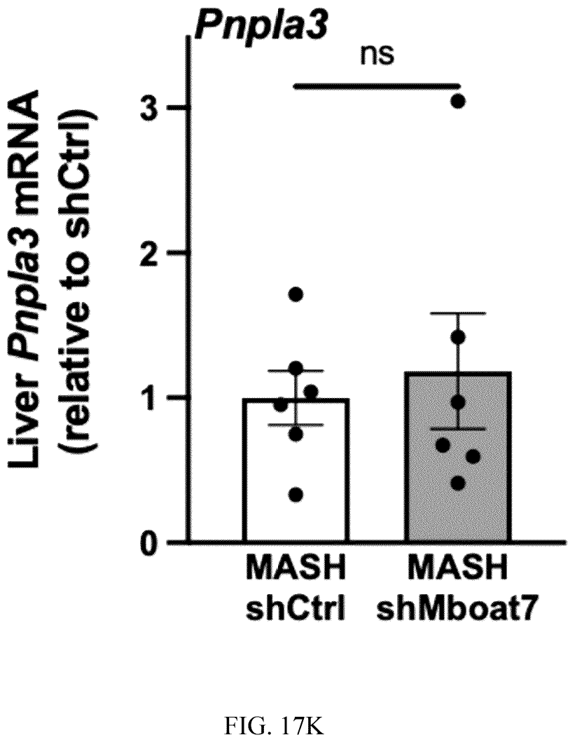

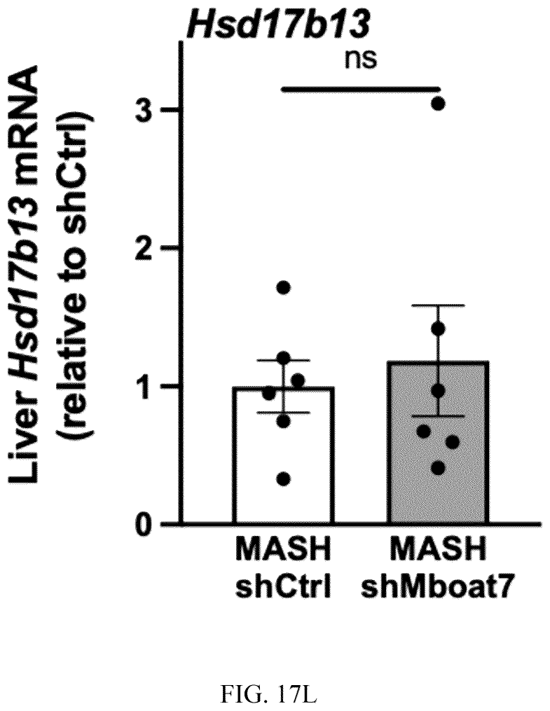

To determine whether further lowering hepatocyte MBOAT7 in mice with hepatosteatosis would exacerbate the development of early MASH fibrosis, 8-week FPC-fed mice were injected with AAV8-H1-shMboat7 to lower hepatocyte-MBOAT7, (Wang X, Zheng Z, Caviglia J M, Corey K E, Herfel T M, Cai B, et al. Hepatocyte TAZ/WWTR1 promotes inflammation and fibrosis in nonalcoholic steatohepatitis. Cell Metab. 2016; 24: 848-62; Wang X, Cai B, Yang X, Sonubi O O, Zheng Z, Ramakrishnan R, et al. Cholesterol stabilizes TAZ in hepatocytes to promote experimental non-alcoholic steatohepatitis. Cell Metab. 2020; 31: 969-986.e967) followed by FPC diet-feeding for 8 additional weeks. Control mice received AAV8-H1-shControl (shCtrl). AAV8-H1-shMboat7 lowered liver MBOAT7 by ˜25% (FIG. 10A). There were no differences in body weight, liver:body weight ratio, or fasting blood sugar (FIGS. 22A-C). Most importantly, hepatocyte-MBOAT7 silencing led to increases in liver fibrosis, fibrosis related mRNAs, and cytokeratin 19 (FIGS. 10B-F and FIG. 17D). Hepatocyte-MBOAT7 silencing showed only a nonsignificant trend toward high Mmp1 but had no effects on Timp2, hepatosteatosis, liver inflammation, plasma ALT, Pnpla3, or Hsd17b13 (FIG. 10G, H and FIGS. 17E-L). These data further support a role for hepatocyte-MBOAT7-LoF in MASH fibrosis.

### Evidence in Hepatocytes, Experimental MASH, and Human Liver Linking MBOAT7 LoF to Elevated TAZ

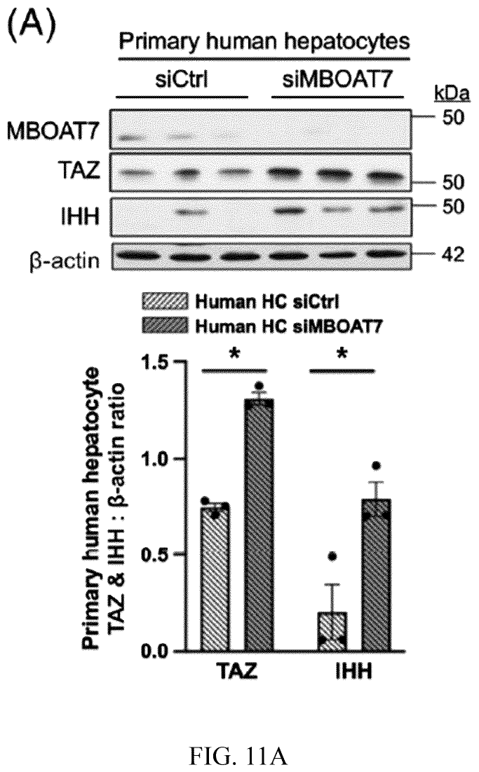

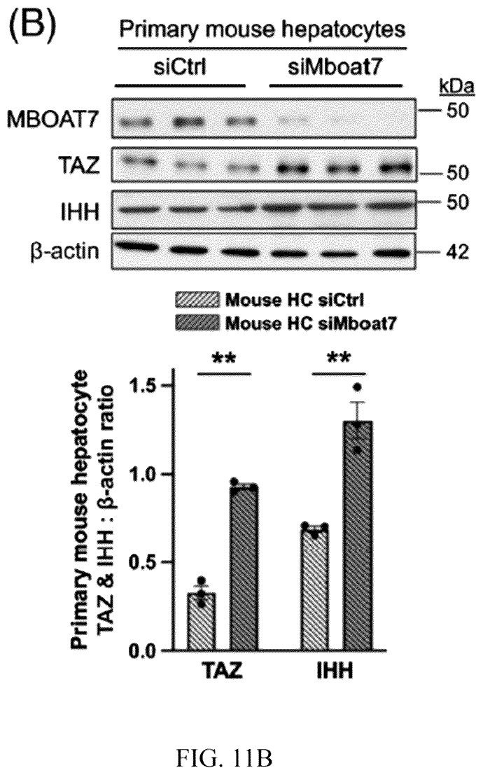

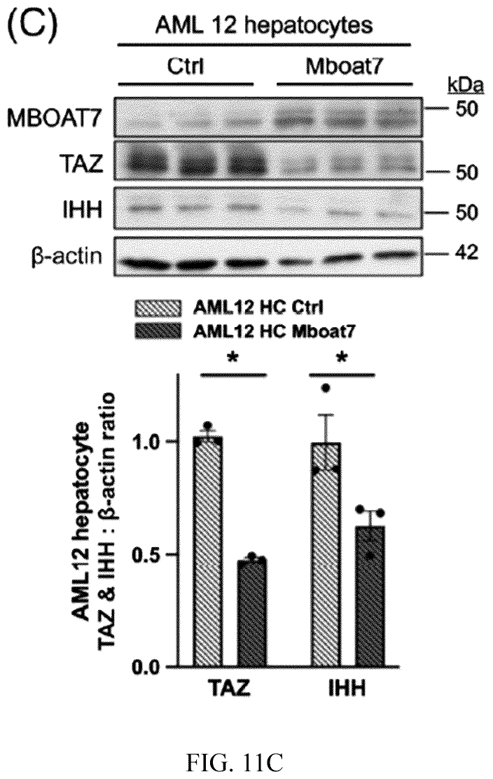

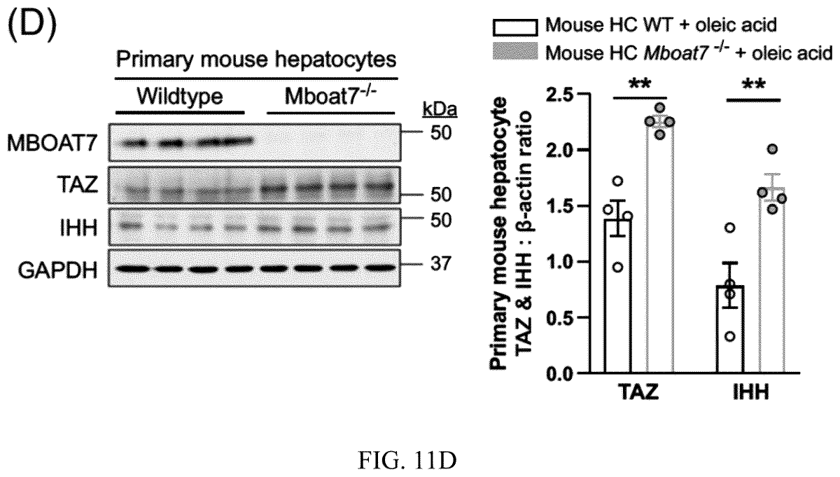

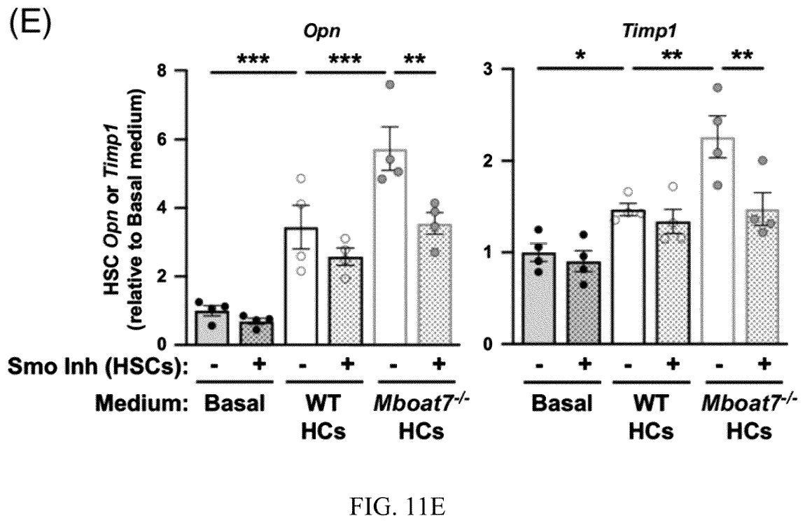

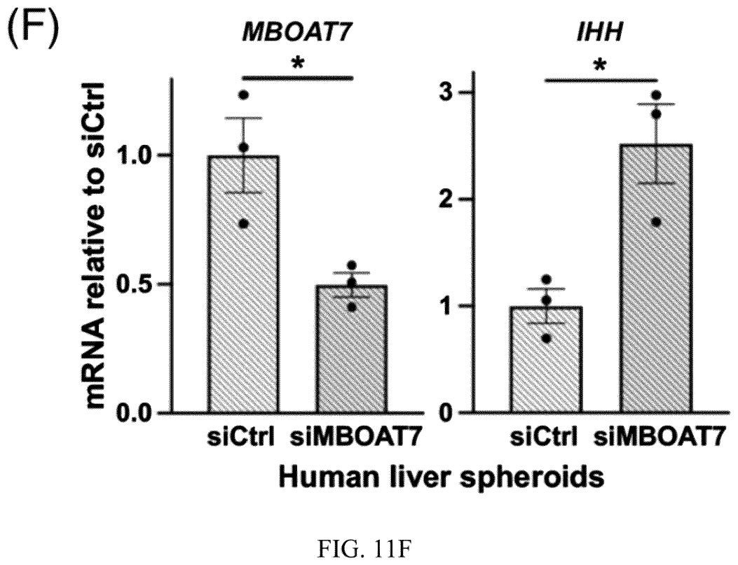

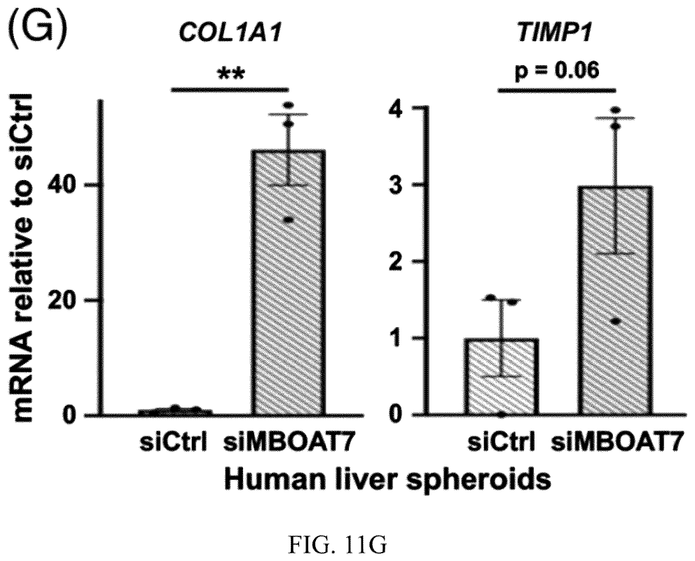

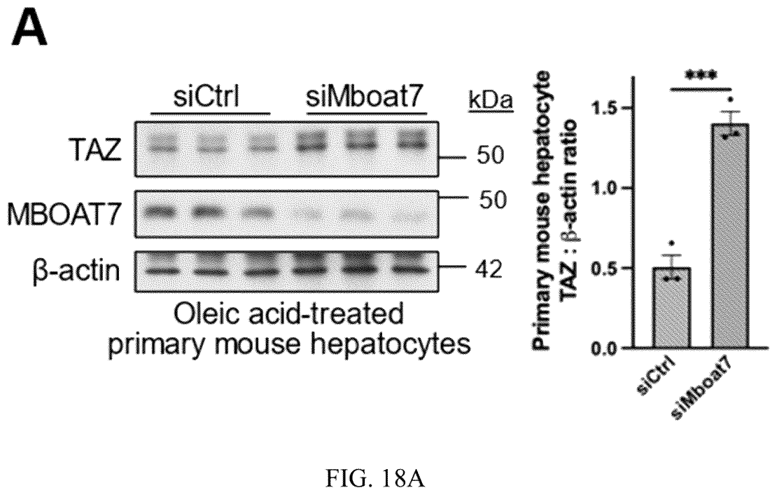

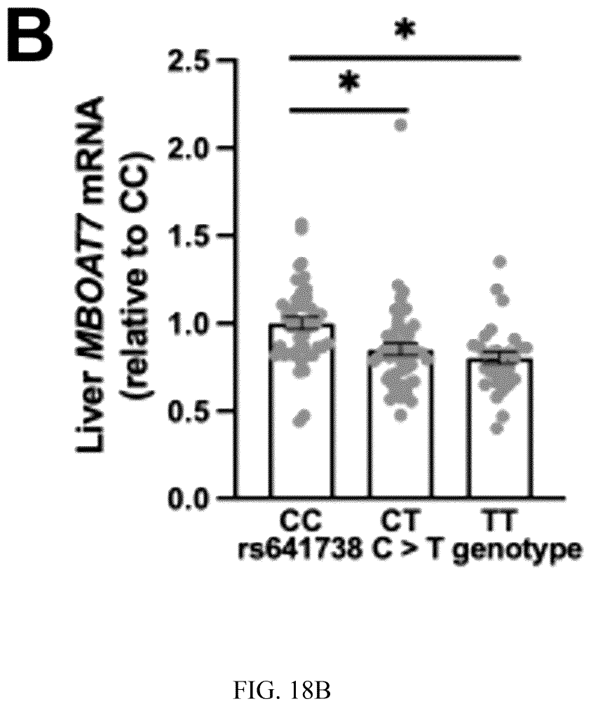

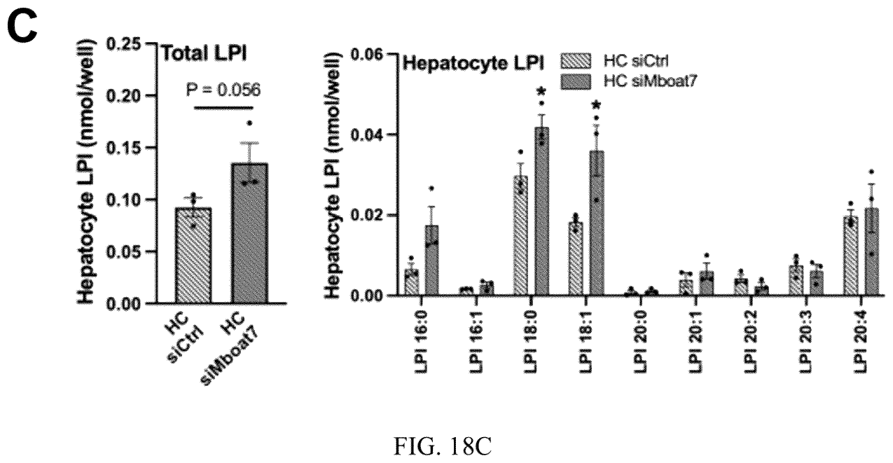

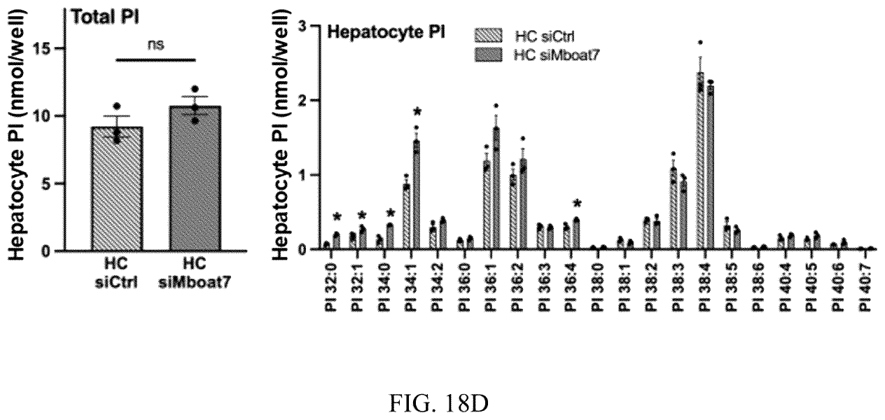

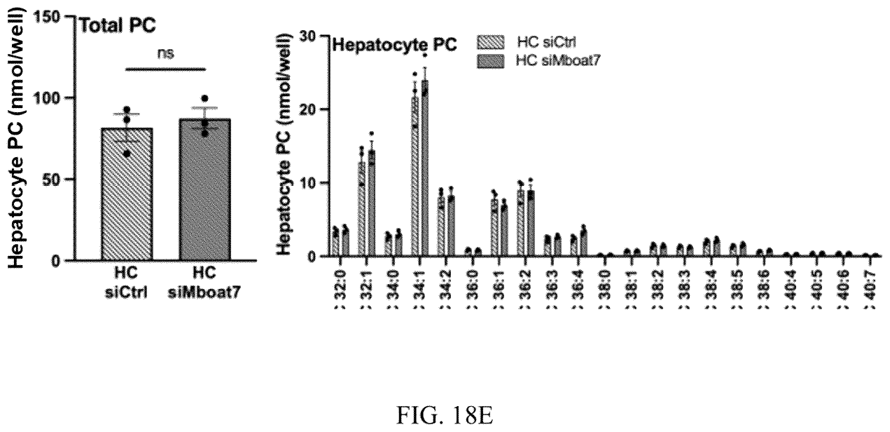

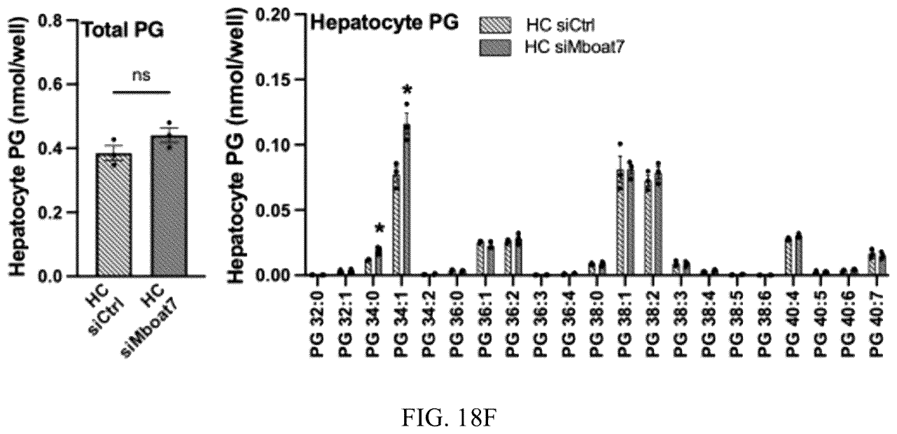

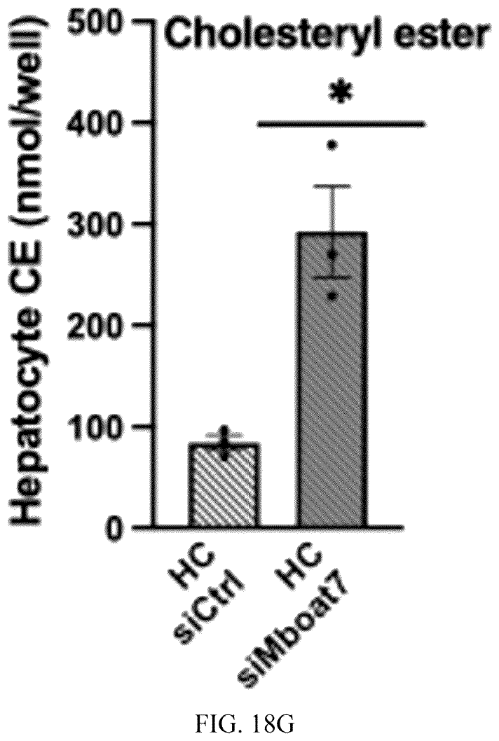

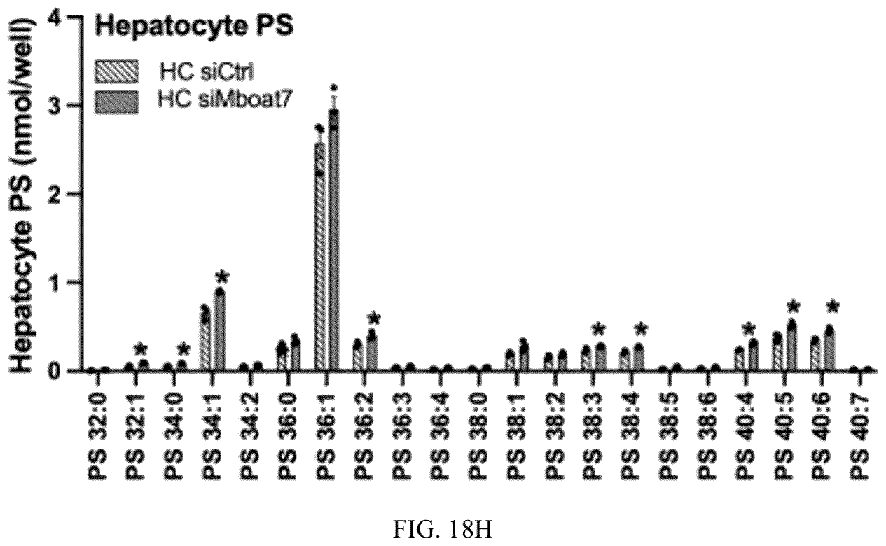

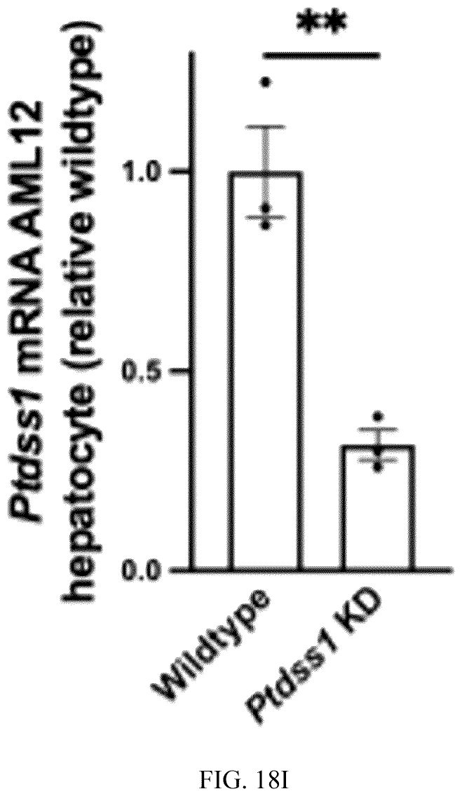

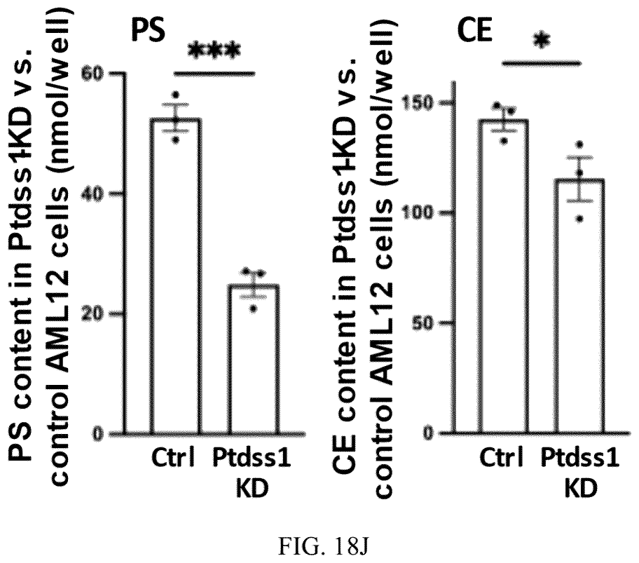

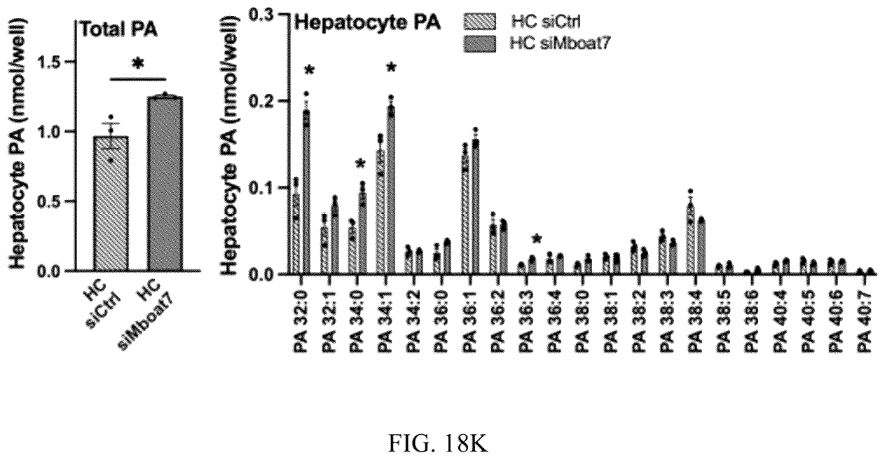

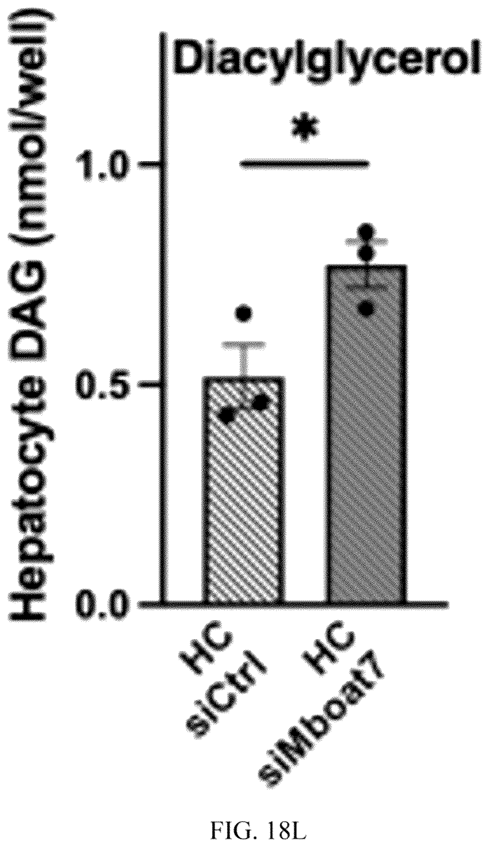

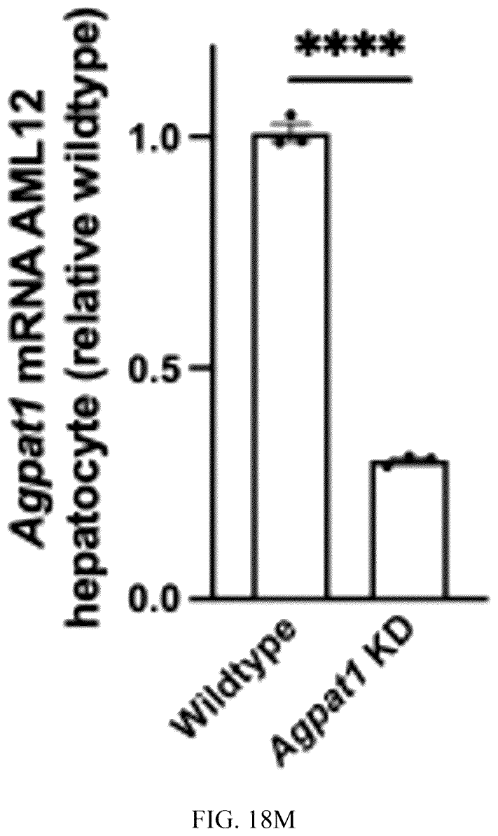

Hepatocyte TAZ is upregulated in human and experimental MASH and, through its gene target IHH, contributes to steatosis-to-fibrotic MASH progression. (Wang X, Zheng Z, Caviglia J M, Corey K E, Herfel T M, Cai B, et al. Hepatocyte TAZ/WWTR1 promotes inflammation and fibrosis in nonalcoholic steatohepatitis. Cell Metab. 2016; 24: 848-62.) In MASH, TAZ protein is stabilized by a pathway involving hepatocyte PM cholesterol trafficking to the cell interior, (Wang X, Cai B, Yang X, Sonubi O O, Zheng Z, Ramakrishnan R, et al. Cholesterol stabilizes TAZ in hepatocytes to promote experimental non-alcoholic steatohepatitis. Cell Metab. 2020; 31: 969-986.e967) which blocks TAZ proteasomal degradation. Given that perturbation in cellular phospholipids (PLs) can alter cholesterol trafficking, (Lagace T A. Phosphatidylcholine: Greasing the cholesterol transport machinery. Lipid Insights. 2015; 8:65-73; Wang B, Tontonoz P. Phospholipid remodeling in physiology and disease. Annu Rev Physiol. 2019; 81:165-88) without being bound by theory MBOAT7 LoF might promote liver fibrosis in part by enhancing the increase in TAZ in MASH. As initial support, partial MBOAT7 silencing in mouse and human primary HCs increased TAZ and the MASH fibrosis mediator, Indian hedgehog (IHH) (Wang X, Zheng Z, Caviglia J M, Corey K E, Herfel T M, Cai B, et al. Hepatocyte TAZ/WWTR1 promotes inflammation and fibrosis in nonalcoholic steatohepatitis. Cell Metab. 2016; 24: 848-62) (FIG. 11A, B), and this effect was also seen when control versus MBOAT7-silenced primary mouse hepatocytes were exposed for 6 hours to oleic acid before assay of TAZ, that is, to reflect the lipid-rich environment of MASH liver (FIG. 18A). Conversely, MBOAT7 transfection of AML12 hepatocytes decreased TAZ and IHH (FIG. 11C).

To relate these findings to HSC activation, the driver of MASH liver fibrosis, a conditioned medium transfer experiment was conducted in which the conditioned medium from control versus MBOAT7-knockout primary hepatocytes, or medium not exposed to cells (basal), was transferred to primary mouse HSCs. For this purpose, oleic acid-treated hepatocytes from Mboat7^{fl/fl} mice (Xia M, Chandrasekaran P, Rong S, Fu X, Mitsche M A. Hepatic deletion of Mboat7 (LPIAT1) causes activation of SREBP-1c and fatty liver. J Lipid Res. 2021; 62:100031) (“wild-type,” WT hepatocytes) or from Mboat7^{fl/fl} mice injected with AAV8-TBG-Cre were used to delete hepatocyte MBOAT7 (Mboat7^{-/-} hepatocytes). Consistent with the above data, the Mboat7^{-/-} hepatocytes showed increases in TAZ and IHH compared with WT hepatocytes (FIG. 11D). Most importantly, the conditioned medium from the Mboat7^{-/-} hepatocytes induced an increase in 2 mRNA markers of HSC activation, Opn and Timp1, compared with medium from WT hepatocytes (FIG. 11E, bars 5 vs. 3 in each graph). As noted above, the increase in hepatocyte TAZ in MASH induces IHH, which is secreted and activates HSCs by activating the Smoothened (Smo) receptor on HSCs. (Wang X, Zheng Z, Caviglia J M, Corey K E, Herfel T M, Cai B, et al. Hepatocyte TAZ/WWTR1 promotes inflammation and fibrosis in nonalcoholic steatohepatitis. Cell Metab. 2016; 24: 848-62.) In this context incubation of the HSCs with a Smo inhibitor neutralized the effect of Mboat7^{-/-} hepatocyte conditioned medium on HSC activation such that Opn and Timp1 expression were similar to the level seen with WT hepatocyte conditioned medium (FIG. 11E, bars 6 vs. 4 and 5 in each graph). Next, an experiment was conducted with control and MBOAT7-silenced human spheroids composed of primary human hepatocytes and nonparenchymal cells, which include HSCs. siMBOAT7 led to ˜50% reduction in MBOAT7, and although there were only enough cells to construct 3 spheroids and assay selected mRNAs, increases in IHH and the HSC activation markers COL1A1 (p<0.01) and TIMP1 (p=0.06) were observed in the MBOAT7-silenced spheroids (FIG. 11F, G). These combined in vitro and ex vivo data support the theory that MBOAT7 LoF promotes the TAZ-IHH HSC activation pathway that contributes to liver fibrosis in MASH.

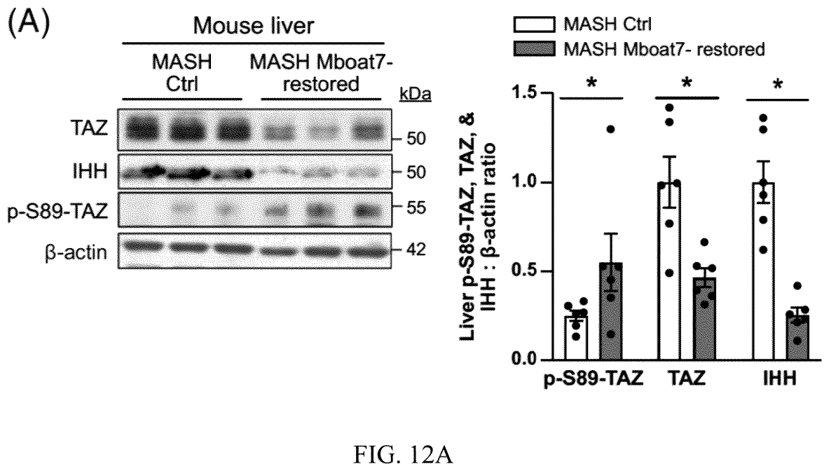

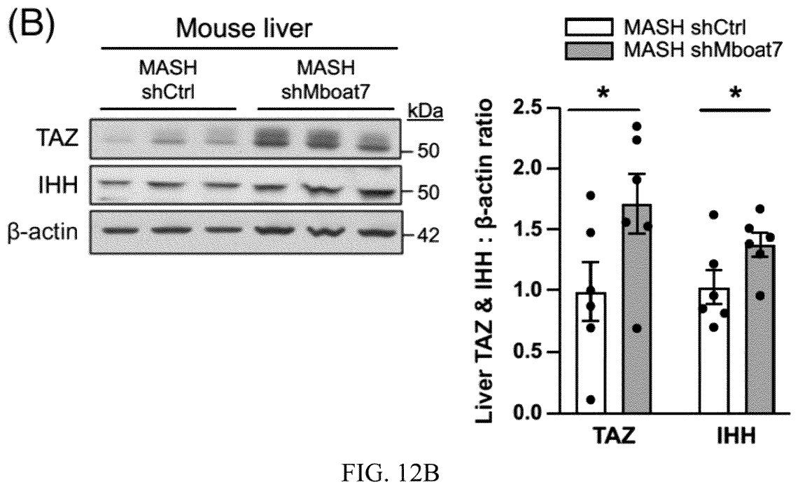

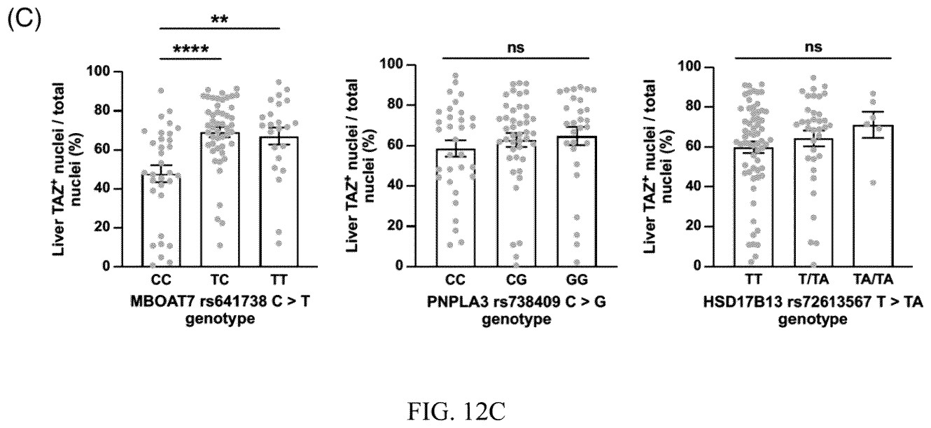

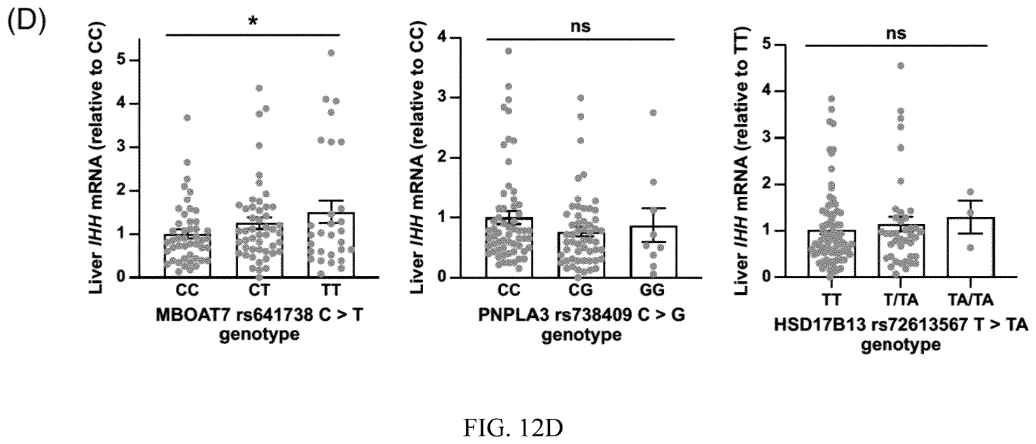

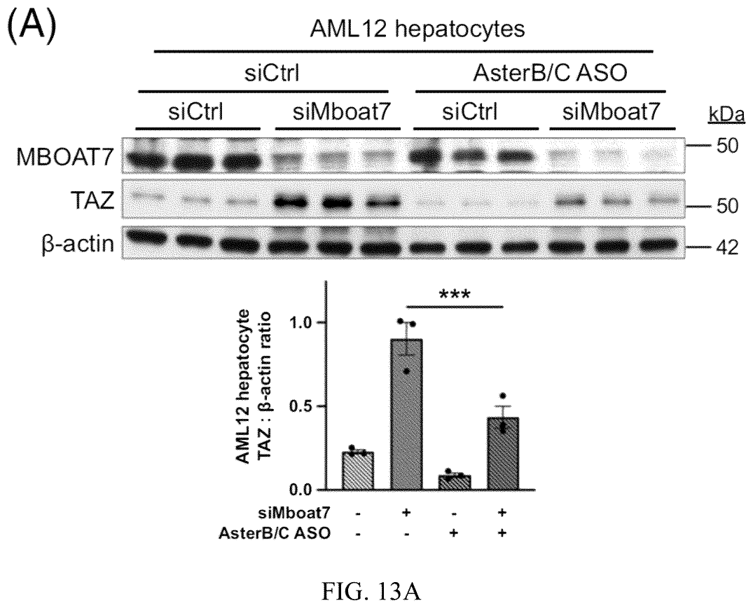

Next, analyses of mouse and human MASH livers were performed. First, the livers of mice with restored hepatocyte-MBOAT7 showed decreased TAZ and IHH (FIG. 12A, blots 1 and 2), while the livers of mice with silenced hepatocyte-MBOAT7 showed increased TAZ and IHH (FIG. 12B). During MASH progression, phosphorylation of TAZ at serine-89 by LATS1/2 is decreased, leading to TAZ stabilization and, thus, IHH induction and HSC activation. (Wang X, Cai B, Yang X, Sonubi O O, Zheng Z, Ramakrishnan R, et al. Cholesterol stabilizes TAZ in hepatocytes to promote experimental non-alcoholic steatohepatitis. Cell Metab. 2020; 31: 969-986.e967) In this context, hepatocyte-MBOAT7 restoration increased p-S89-TAZ (FIG. 12A, blot 3), that is, destabilized TAZ, consistent with the idea that hepatocyte MBOAT7 is protective against the MASH-TAZ pathway elucidated in the previous studies. (Wang X, Zheng Z, Caviglia J M, Corey K E, Herfel T M, Cai B, et al. Hepatocyte TAZ/WWTR1 promotes inflammation and fibrosis in nonalcoholic steatohepatitis. Cell Metab. 2016; 24: 848-62; Wang X, Cai B, Yang X, Sonubi O O, Zheng Z, Ramakrishnan R, et al. Cholesterol stabilizes TAZ in hepatocytes to promote experimental non-alcoholic steatohepatitis. Cell Metab. 2020; 31: 969-986.e967) Turning to humans, MASH liver sections from the FLINT study (Neuschwander-Tetri B A, Loomba R, Sanyal A J, Lavine J E, Van Natta M L, Abdelmalek M F, et al. Farnesoid X nuclear receptor ligand obeticholic acid for non-cirrhotic, non-alcoholic steatohepatitis (FLINT): A multicentre, randomised, placebo-controlled trial. Lancet. 2015; 385:956-65) were immunostained for nuclear (active) TAZ, comparing individuals with the rs641738-T (risk allele) versus the rs641738-C (nonrisk allele) (FIG. 20). The risk allele was associated with higher nuclear-TAZ compared to those without the risk allele, whereas there was no correlation of nuclear-TAZ with 2 other common MASH-risk variants, PNPLA3 rs738409-G and HSD17B13 rs72613567-TA (FIG. 12C). In a separate cohort of obese individuals FIG. 21), the rs641738-T risk allele, which was associated with lower hepatic MBOAT7 as expected (Buch S, Stickel F, Trepo E, Way M, Herrmann A, Nischalke H D, et al. A genome-wide association study confirms PNPLA3 and identifies TM6SF2 and MBOAT7 as risk loci for alcohol-related cirrhosis. Nat Genet. 2015; 47:1443-8) (FIG. 18B), had higher expression of IHH mRNA compared with those who did not carry this allele, with no effect of PNPLA3 rs738409-G or HSD17B13 rs72613567-TA (FIG. 12D).

### MBOAT7 LoF in Hepatocytes Enhances the Cholesterol Trafficking-Activated TAZ Pathway

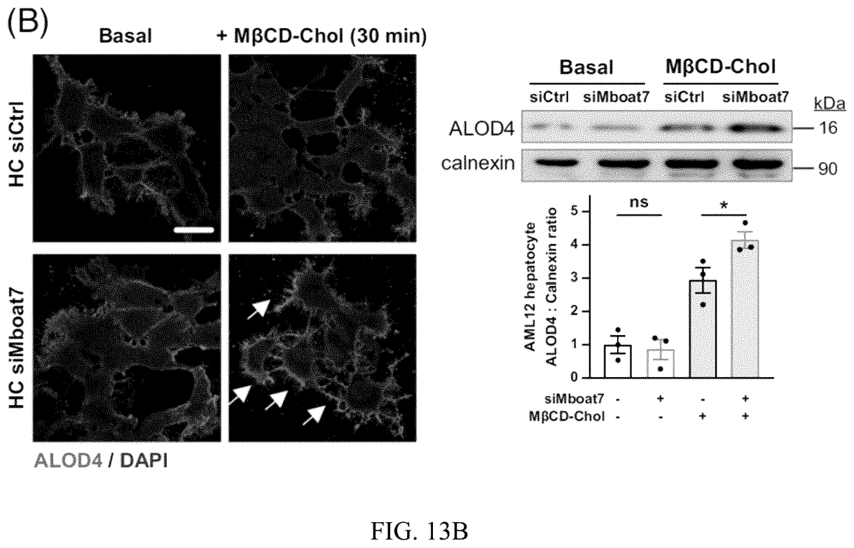

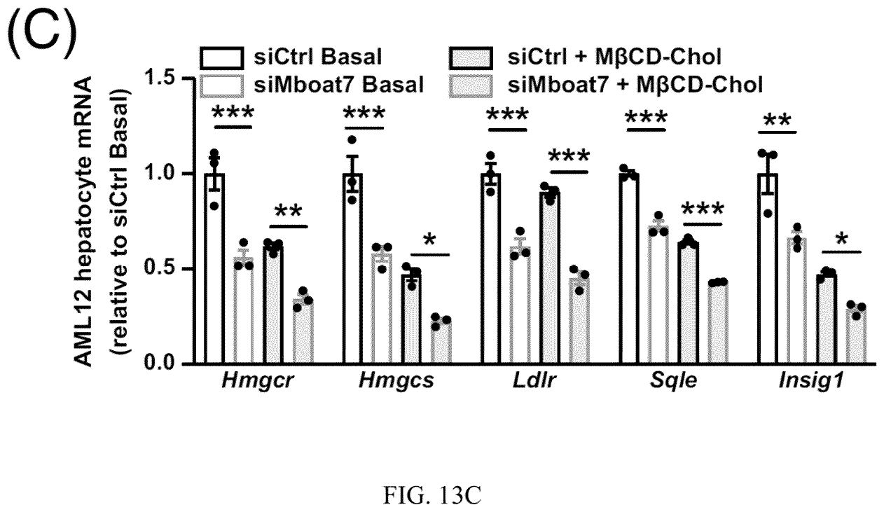

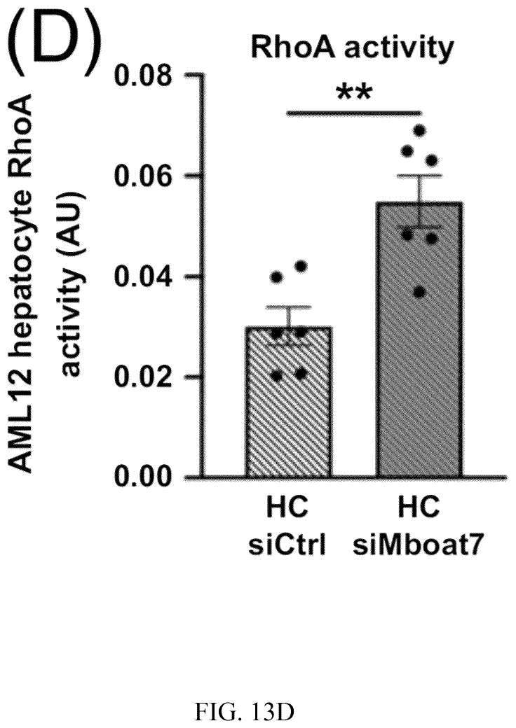

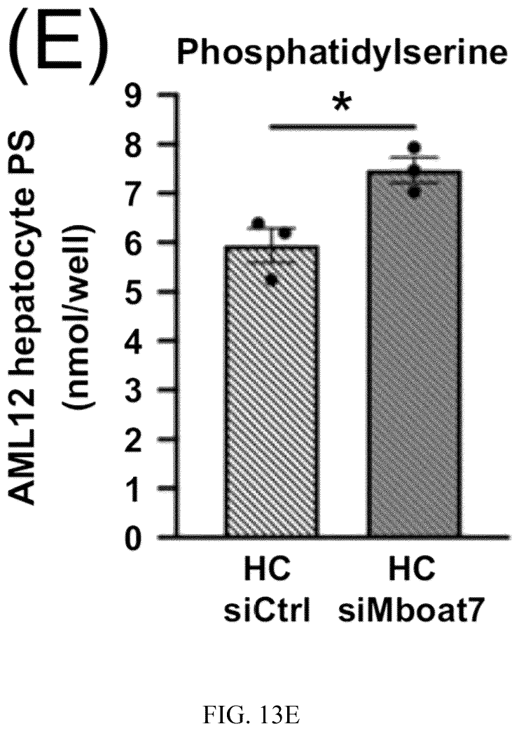

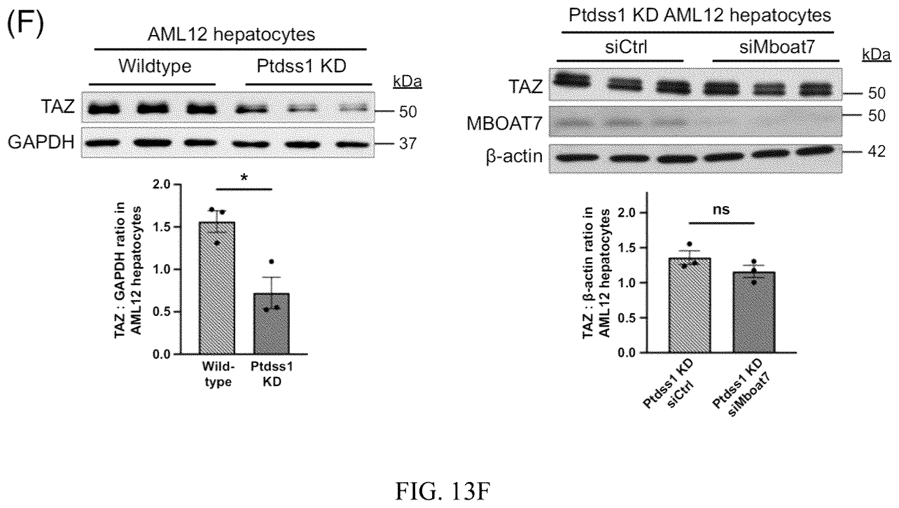

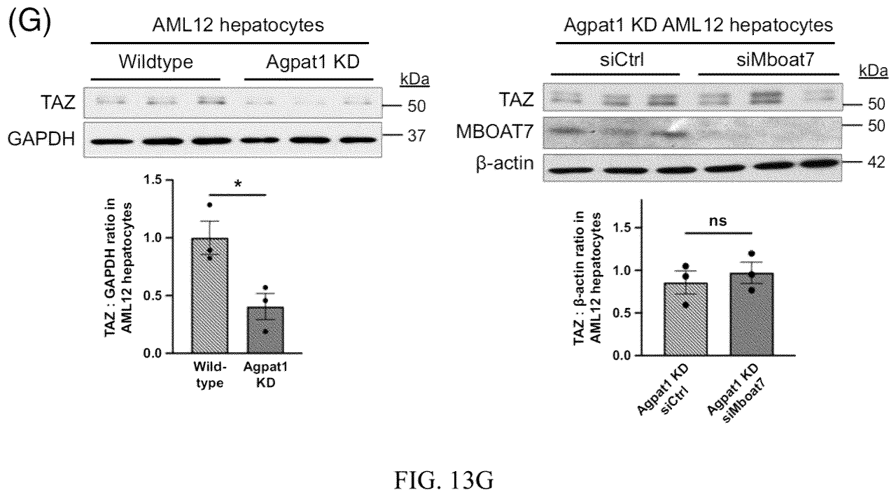

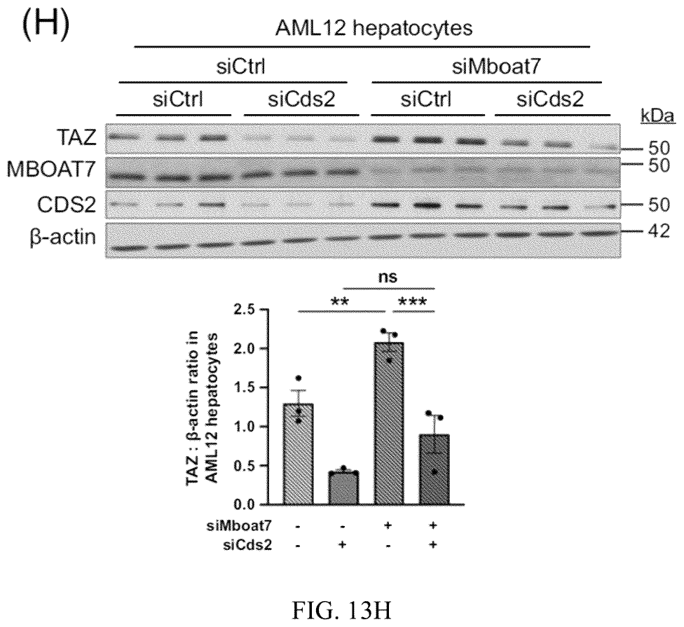

The increase in PM cholesterol trafficking in MASH, the first step in TAZ upregulation, is mediated by the cholesterol trafficking proteins ASTER-B/C. (Wang X, Cai B, Yang X, Sonubi O O, Zheng Z, Ramakrishnan R, et al. Cholesterol stabilizes TAZ in hepatocytes to promote experimental non-alcoholic steatohepatitis. Cell Metab. 2020; 31: 969-986.e967). In this context, the siMboat7-induced increase in TAZ in AML12 hepatocytes was blunted when ASTERB/C was silenced (FIG. 13A). ASTER proteins interact specifically with a pool of accessible cholesterol in the PM to mediate trafficking. (Sandhu J, Li S, Fairall L, Pfisterer S G, Gurnett J E, Xiao X, et al. Aster proteins facilitate nonvesicular plasma membrane to ER cholesterol transport in mammalian cells. Cell. 2018; 175:514-529.e520). As changes in PM PLs can alter cholesterol trafficking (Lange Y, Ye J, Steck T L. How cholesterol homeostasis is regulated by plasma membrane cholesterol in excess of phospholipids. Proc Natl Acad Sci USA. 2004; 101:11664-7), the next question was if MBOAT7 LoF affected PM cholesterol accessibility. Accessible cholesterol in the PM, but not inaccessible cholesterol, interacts with a peptide encompassing domain 4 of anthrolysin O called ALOD4 (Johnson K A, Radhakrishnan A. The use of anthrolysin O and ostreolysin A to study cholesterol in cell membranes. Methods Enzymol. 2021; 649:543-66), and accessible cholesterol can be monitored by immunofluorescence microscopy using His6-tagged ALOD4 (“His6” disclosed as SEQ ID NO: 70). (Ferrari A, He C, Kennelly J P, Sandhu J, Xiao X, Chi X, et al. Aster proteins regulate the accessible cholesterol pool in the plasma membrane. Mol Cell Biol) As predicted, a higher binding of ALOD4 to the PM of siMboat7-treated AML12 hepatocytes was observed compared with siCtrl-treated cells, which was most evident at 30 minutes following cholesterol loading with MβCD-cholesterol, and cell-binding of His6-ALOD4 (“His6” disclosed as SEQ ID NO: 70) as measured by immunoblot was also increased by siMboat7 in cholesterol-treated AML12 cells (FIG. 13B). According to the theory, these data indicate an early increase in PM cholesterol accessibility, which would then lead to increased cholesterol trafficking to the cell interior. Consistent with this idea, 5 mRNAs that are downregulated by cholesterol trafficking to the cell interior, Hmgcr, Hmgcs, Ldlr, Sqle, and Insig1, were markedly lower in both basal and cholesterol-treated AML12 cells following MBOAT7-silencing (FIG. 13C). Moreover, a key step linking hepatocyte cholesterol trafficking to increased TAZ in MASH is RhoA activation (Wang X, Cai B, Yang X, Sonubi O O, Zheng Z, Ramakrishnan R, et al. Cholesterol stabilizes TAZ in hepatocytes to promote experimental non-alcoholic steatohepatitis. Cell Metab. 2020; 31: 969-986.e967) and RhoA activity in hepatocytes was increased by MBOAT7-silencing (FIG. 13D). These combined data suggest that MBOAT7 LoF promotes PM-cholesterol accessibility and trafficking to increase TAZ, providing a plausible link between MBOAT7-LoF and MASH fibrosis. (Wang X, Zheng Z, Caviglia J M, Corey K E, Herfel T M, Cai B, et al. Hepatocyte TAZ/WWTR1 promotes inflammation and fibrosis in nonalcoholic steatohepatitis. Cell Metab. 2016; 24:848-62; Wang X, Sommerfeld M R, Jahn-Hofmann K, Cai B, Filliol A, Remotti H E, et al. A therapeutic silencing RNA targeting Hepatocyte TAZ prevents and reverses fibrosis in nonalcoholic steatohepatitis in mice. Hepatol Commun. 2019; 3:1221-34; Wang X, Cai B, Yang X, Sonubi O O, Zheng Z, Ramakrishnan R, et al. Cholesterol stabilizes TAZ in hepatocytes to promote experimental non-alcoholic steatohepatitis. Cell Metab. 2020; 31: 969-986.e967; Wang X, Moore M P, Shi H, Miyata Y, Donnelly S K, Radiloff D R, et al. Hepatocyte-targeted siTAZ therapy lowers liver fibrosis in NASH diet-fed chimeric mice with hepatocyte-humanized livers. Mol Ther Methods Clin Dev. 2023; 31:101165).

### The Increase in TAZ in MBOAT7-Silenced Hepatocytes is Linked to a PL Metabolism Pathway

Based on the theory that MBOAT7-LoF-induced changes in PLs are responsible for increasing the cholesterol trafficking pathway that upregulates TAZ, the PLs of MBOAT7-silenced versus control AML12 hepatocytes were analyzed. As predicted (Thangapandi V R, Knittelfelder O, Brosch M, Patsenker E, Vvedenskaya O, Buch S, et al. Loss of hepatic Mboat7 leads to liver fibrosis. Gut. 2021; 70:940-50; Helsley R N, Varadharajan V, Brown A L, Gromovsky A D, Schugar R C, Ramachandiran I, et al. Obesity-linked suppression of membrane-bound O-acyltransferase 7 (MBOAT7) drives nonalcoholic fatty liver disease. eLife. 2019; 8:e49882), LPI 18:0 and 18:1 were higher in MBOAT7-silenced hepatocytes, with no change in total PI, phosphatidylglycerol, or phosphatidylcholine (PC) (FIG. 18C-F). MBOAT7-silenced hepatocytes also had increases in cholesteryl ester (FIG. 18G), consistent with increased PM-to-endoplasmic reticulum cholesterol trafficking (Tabas I, Rosoff W J, Boykow G C. Acyl coenzyme A:cholesterol acyl transferase in macrophages utilizes a cellular pool of cholesterol oxidase-accessible cholesterol as substrate. J Biol Chem. 1988; 263:1266-72), and phosphatidylserine (PS) (FIG. 13E and FIG. 18H). The PS data were particularly interesting, as PS mediates ASTER recruitment to PM-endoplasmic reticulum contact sites to facilitate cholesterol trafficking, (Sandhu J, Li S, Fairall L, Pfisterer S G, Gumett J E, Xiao X, et al. Aster proteins facilitate nonvesicular plasma membrane to ER cholesterol transport in mammalian cells. Cell. 2018; 175:514-529.e520) and deletion of the PS-synthesizing enzyme PS synthase-1 (PSS1) impedes ASTER-mediated cholesterol binding and subsequent internalization into cells. (Trinh M N, Brown M S, Seemann J, Vale G, McDonald J G, Goldstein J L, et al. Interplay between Asters/GRAMD1s and phosphatidylserine in intermembrane transport of LDL cholesterol. Proc Natl Acad Sci. 2022; 119:e2120411119) In this context, PSS1 (Ptdss1) deletion by CRISPR/Cas9, which lowered cellular PS as designed and also lowered cellular cholesteryl ester as an indicator of reduced cholesterol trafficking, decreased TAZ expression under both basal and MBOAT7-silenced AML12 cells (FIG. 13F and FIG. 18I). The role of PSS1 in basal TAZ expression is consistent with the data in FIG. 13A that ASTER-B/C ASO lowers basal TAZ in AML12 cells, but the key finding is that PSS1 is involved in TAZ upregulation in the setting of MBOAT7-LoF.

PSS1 converts PC into PS. (Sturbois-Balcerzak B, Stone S J, Sreenivas A, Vance J E. Structure and expression of the murine phosphatidylserine synthase-1 gene. J Biol Chem. 2001; 276:8205-12) Based on previous work showing that enhanced PI turnover resulting from MBOAT7 deletion in hepatocytes leads to an increase in diacylglycerol (DAG) (Tanaka Y, Shimanaka Y, Caddeo A, Kubo T, Mao Y, Kubota T, et al. LPIAT1/MBOAT7 depletion increases triglyceride synthesis fueled by high phosphatidylinositol turnover. Gut. 2021; 70: 180-93.) and that DAG can be converted into PC, (Kennedy E P, Weiss S B. The function of cytidine coenzymes in the biosynthesis of phospholipides. J Biol Chem. 1956; 222:193-214) without being bound by theory the following pathway in MBOAT-LoF hepatocytes: lysophosphatidic acid→phosphatidic acid (PA)→CDP-DAG→PI→DAG→PC→PS→cholesterol trafficking→TAZ. PA is synthesized from lysophosphatidic acid through 1-acylglycerol-3-phosphate O-acyltransferase 1 (AGPAT1), and PA is converted to CDP-DAG through CDP-DAG synthase-2 (CDS2), leading to increased PI through PI synthase and then DAG through a PLC-like activity. (Tanaka Y, Shimanaka Y, Caddeo A, Kubo T, Mao Y, Kubota T, et al. LPIAT1/MBOAT7 depletion increases triglyceride synthesis fueled by high phosphatidylinositol turnover. Gut. 2021; 70: 180-93) Consistent with the predicted pathway, MBOAT7-silenced hepatocytes had an increase in both PA and DAG (FIG. 18K, L). Both AGAPT1-knockdown and CDS2-silencing lowered basal TAZ and, most importantly, prevented the increment in TAZ caused by silencing MBOAT7 (FIG. 13G, H and FIG. 18M). These data provide causation evidence for the predicted AGPAT1-CDS2-PSS1 pathway linking MBOAT7-LoF to increased TAZ.

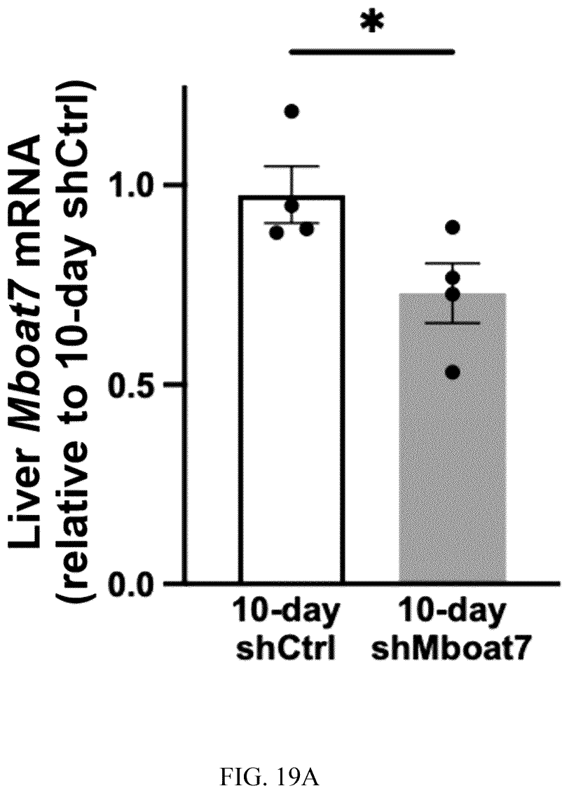

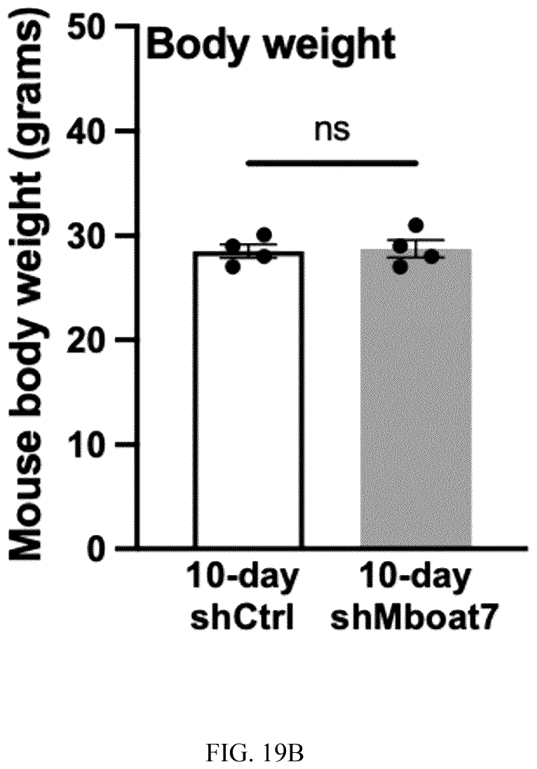

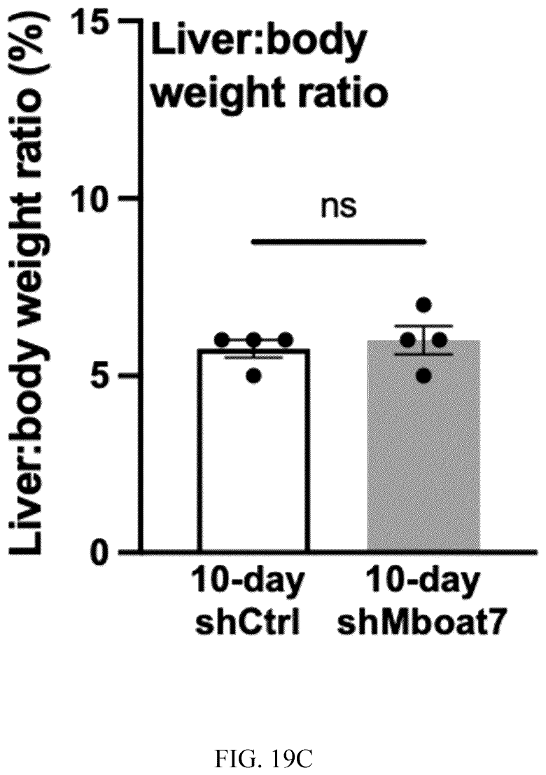

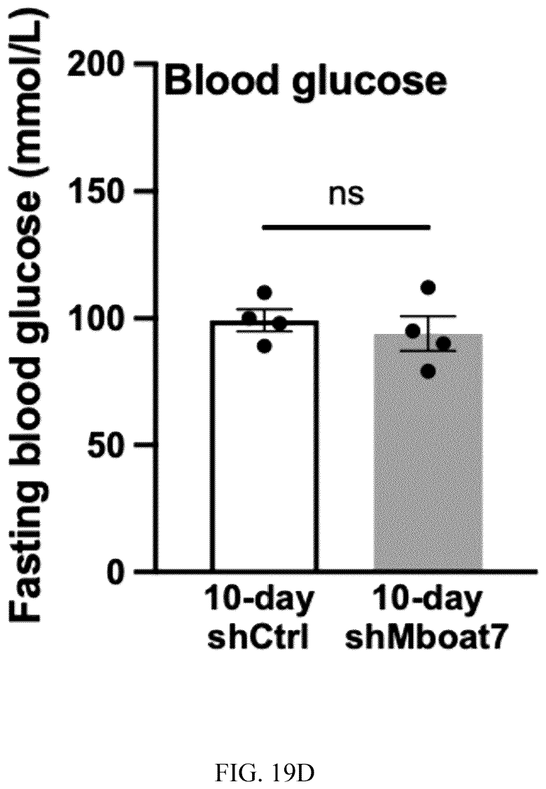

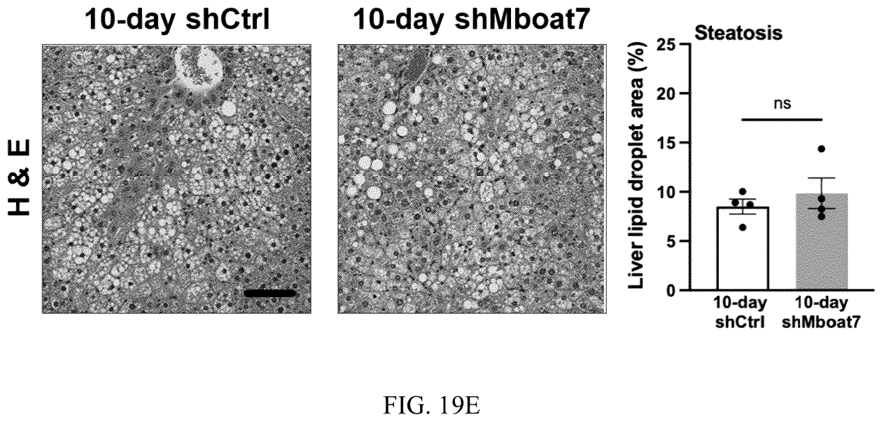

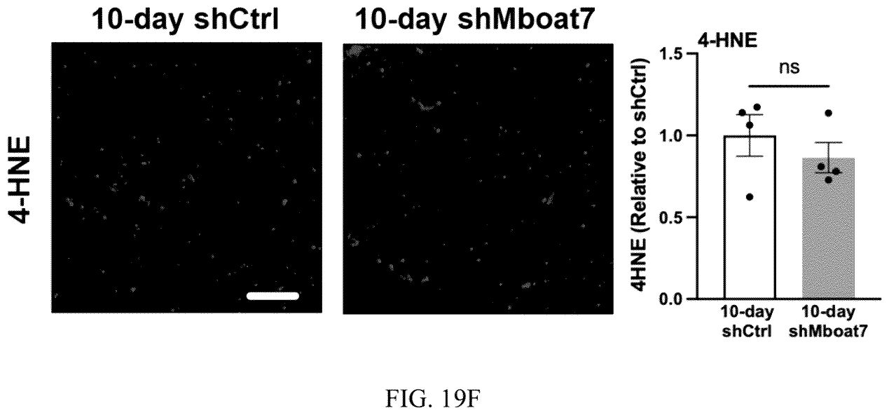

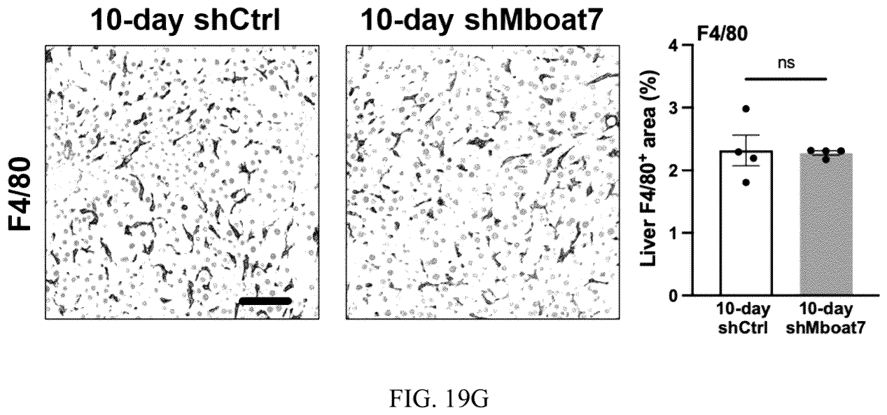

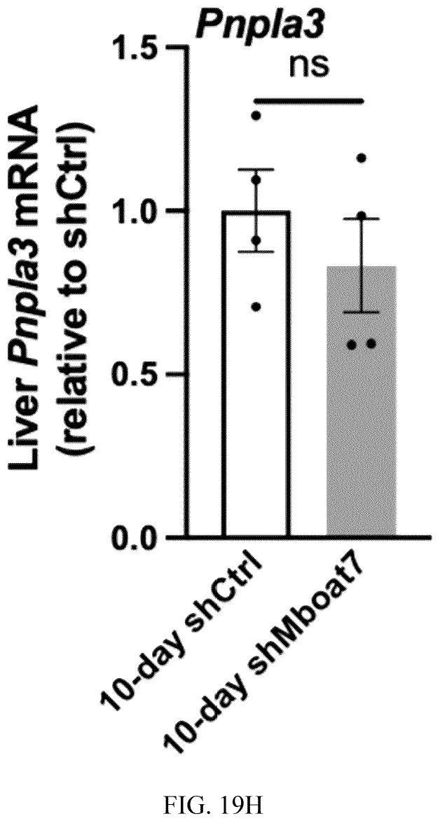

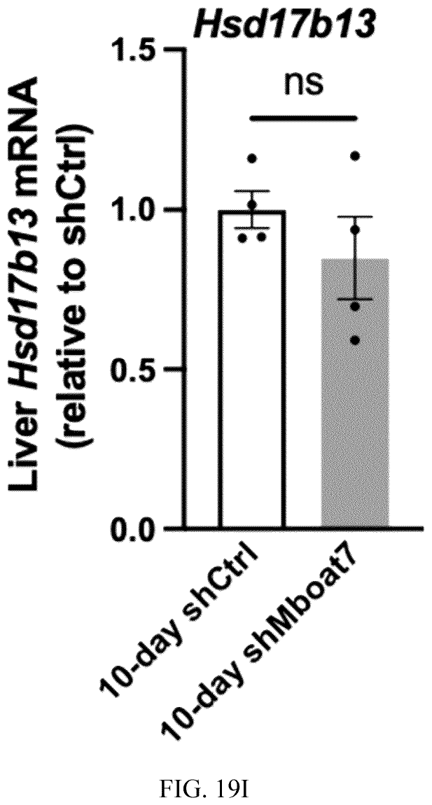

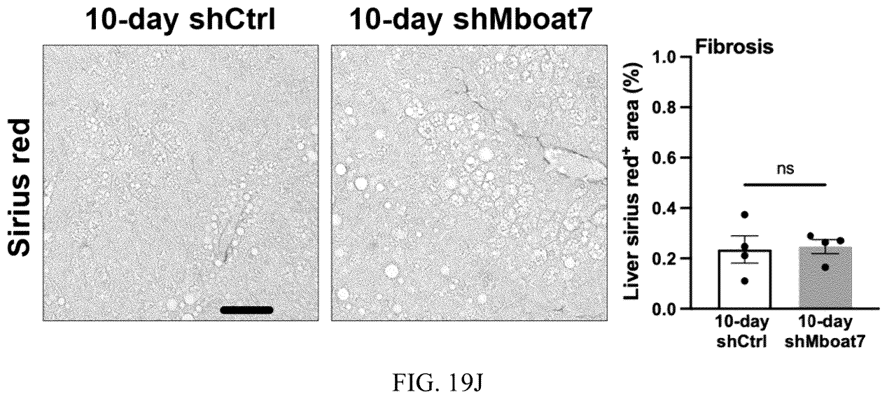

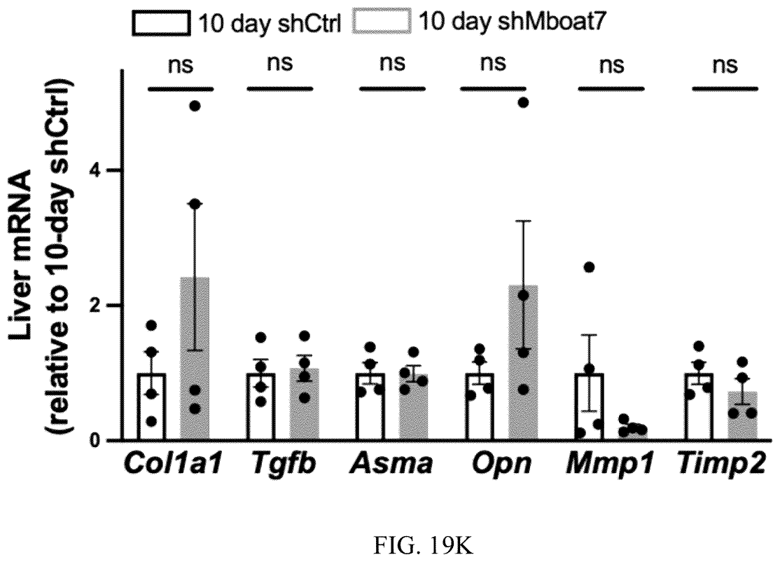

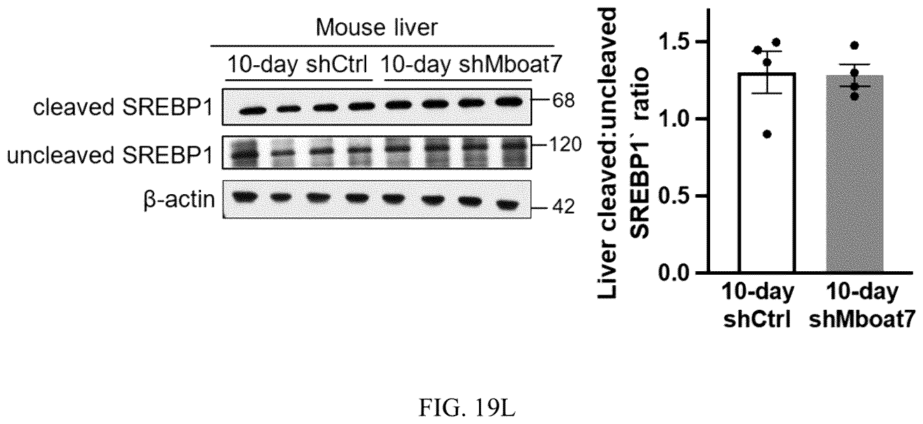

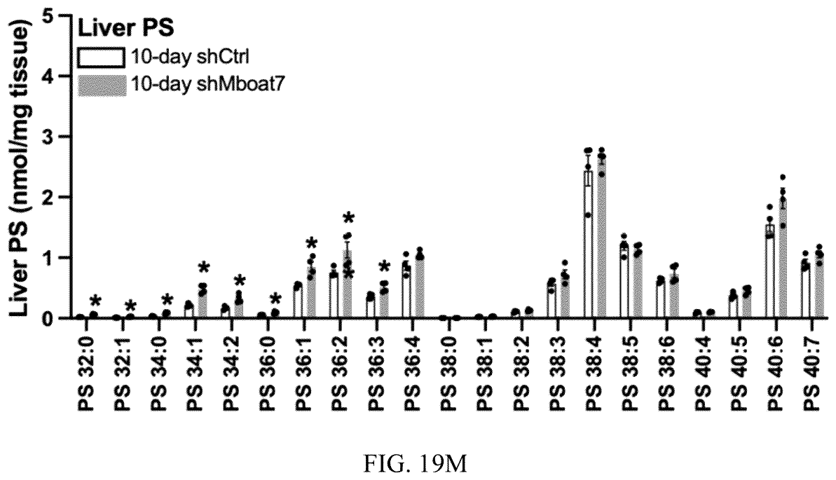

To seek biochemical evidence of the pathway in vivo, mice with established hepatosteatosis were given AAV8-H1-shMboat7 or control virus and then harvested 10 days later to capture early changes. Similar to the longer-term experiment in FIG. 10, Mboat7 mRNA was ˜25% lower in the AAV8-H1-shMboat7-treated mice (FIG. 19A), and there were no differences in body weight, liver:body weight ratio, fasting blood glucose, liver lipid droplet area, liver 4-HNE staining as a marker of oxidative stress, liver macrophages, or expression of Pnpla3 or Hsd17b13 (FIG. 19B-I). At this early time point, there was a modest decrease in liver phospho-TAZ (above) and an increase in liver TAZ in the hepatocyte-MBOAT7-silenced mice (FIG. 14A) and, as designed, no increase yet in liver fibrosis or mRNA markers of HSC activation (FIG. 19J, K). Most importantly, the livers of the hepatocyte-MBOAT7-silenced cohort showed decreased cleavage of SREBP2 (FIG. 14B) but not SREBP1 (FIG. 19L) and decreased expression of 4 sterol responsive mRNAs-Srebp2, Hmgcs, Ldlr, and Insig1 (FIG. 14C), consistent with increased cholesterol trafficking to the cell interior of hepatocytes. The livers of the AAV8-H1-shMboat7-treated mice also had higher contents of PS and DAG (FIG. 14D, E and FIG. 19M), in line with the theory on how MBOAT7 deficiency is linked to increased PS (above). Finally, MBOAT7-silenced livers also showed a higher pCREB: total CREB ratio, which marks a key upstream step in the cholesterol trafficking-TAZ pathway (Wang X, Cai B, Yang X, Sonubi O O, Zheng Z, Ramakrishnan R, et al. Cholesterol stabilizes TAZ in hepatocytes to promote experimental non-alcoholic steatohepatitis. Cell Metab. 2020; 31: 969-986.e967.) (FIG. 14F). These data, when combined with those above, support the theory that MBOAT7-LoF promotes a cholesterol trafficking pathway that increases profibrotic TAZ in MASH (FIG. 14G).

### Discussion

Interest in MBOAT7 in MASLD/MASH stems from the findings that 2 independent risk factors for MASLD/MASH—obesity and a common polymorphism, rs641738 C>T (and variants in LD with it in that locus)—are associated with decreased expression of liver MBOAT7. (Busca C, Arias P, Sánchez-Conde M, Rico M, Montejano R, Martin-Carbonero L, et al. Genetic variants associated with steatohepatitis and liver fibrosis in HIV-infected patients with NAFLD. Front Pharmacol. 2022; 13:905126; Dongiovanni P, M Anstee Q, Valenti L. Genetic predisposition in NAFLD and NASH: Impact on severity of liver disease and response to treatment. Curr Pharmaceut Des. 2013; 19:5219-38; Krawczyk M, Rau M, Schattenberg J M, Bantel H, Pathil A, Demir M, et al. Combined effects of the PNPLA3 rs738409, TM6SF2 rs58542926, and MBOAT7 rs641738 variants on NAFLD severity: A multicenter biopsy-based study. J Lipid Res. 2017; 58:247-55; Luukkonen P K, Zhou Y, Hyötyläinen T, Leivonen M, Arola J, Orho-Melander M, et al. The MBOAT7 variant rs641738 alters hepatic phosphatidylinositols and increases severity of nonalcoholic fatty liver disease in humans. J Hepatol. 2016; 65:1263-5; Mancina R M, Dongiovanni P, Petta S, Pingitore P, Meroni M, Rametta R, et al. The MBOAT7-TMC4 variant rs641738 increases risk of nonalcoholic fatty liver disease in individuals of European descent. Gastroenterology. 2016; 150:1219-230. e1216; Meroni M, Longo M, Fracanzani A L, Dongiovanni P. MBOAT7 down-regulation by genetic and environmental factors predisposes to MAFLD. EBioMedicine. 2020; 57:102866; Pazoki R, Vujkovic M, Elliott J, Evangelou E, Gill D, Ghanbari M, et al. Genetic analysis in European ancestry individuals identifies 517 loci associated with liver enzymes. Nat Commun. 2021; 12:2579; Raja A M, Ciociola E, Ahmad I N, Dar F S, Naqvi S M S, Moaeen-Ud-Din M, et al. Genetic susceptibility to chronic liver disease in individuals from Pakistan. Int J Mol Sci.; Teo K, Abeysekera K W M, Adams L, Aigner E, Anstee Q M, Banales J M, et al. rs641738C>T near MBOAT7 is associated with liver fat, ALT and fibrosis in NAFLD: A meta-analysis. J Hepatol. 2021; 74:20-30; Thangapandi V R, Knittelfelder O, Brosch M, Patsenker E, Vvedenskaya O, Buch S, et al. Loss of hepatic Mboat7 leads to liver fibrosis. Gut. 2021; 70:940-50; Xu X, Xu H, Liu X, Zhang S, Cao Z, Qiu L, et al. MBOAT7 rs641738 (C>T) is associated with NAFLD progression in men and decreased ASCVD risk in elder Chinese population. Front Endocrinol (Lausanne). 2023; 14:1199429; Chen Y, Du X, Kuppa A, Feitosa M F, Bielak L F, O'Connell J R, et al. Genome-wide association meta-analysis identifies 17 loci associated with nonalcoholic fatty liver disease. Nat Genet. 2023; 55:1640-50; Helsley R N, Varadharajan V, Brown A L, Gromovsky A D, Schugar R C, Ramachandiran I, et al. Obesity-linked suppression of membrane-bound O-acyltransferase 7 (MBOAT7) drives nonalcoholic fatty liver disease. eLife. 2019; 8:e49882). As rs641738 C>T has been associated with both hepatosteatosis and fibrosis and because hepatosteatosis can promote liver fibrosis (Wree A, Broderick L, Canbay A, Hoffman H M, Feldstein A E. From NAFLD to NASH to cirrhosis-new insights into disease mechanisms. Nat Rev Gastroenterol Hepatol. 2013; 10: 627-36; Bohinc B N, Diehl A M. Mechanisms of disease progression in NASH: New paradigms. Clin Liver Dis. 2012; 16:549-65), a key gap is whether MBOAT7 LoF can directly promote liver fibrosis, the most important determinant of clinical outcomes in MASH. (Angulo P, Kleiner D E, Dam-Larsen S, Adams L A, Bjornsson E S, Charatcharoenwitthaya P, et al. Liver fibrosis, but no other histologic features, is associated with long-term outcomes of patients with nonalcoholic fatty liver disease. Gastroenterology. 2015; 149:389-397.e310.). Described herein is that silencing hepatocyte MBOAT7 after the development of hepatosteatosis exacerbated the progression to early liver fibrosis without worsening steatosis. Conversely, restoring hepatocyte MBOAT7 in mice with established diet induced hepatosteatosis, which lowers hepatocyte MBOAT7 even in the absence of rs641738 C>T, decreased the progression to early liver fibrosis without improving steatosis. These results support the concept that MBOAT7 LoF can directly promote MASH liver fibrosis.

Based on these findings, how MBOAT7 LoF in hepatocytes could promote liver fibrosis was determined. This effort led to a theory based on the PL remodeling function of MBOAT7 (Anderson K E, Kielkowska A, Durrant T N, Juvin V, Clark J, Stephens L R, et al. Lysophosphatidylinositol-acyltransferase-1 (LPIAT1) is required to maintain physiological levels of PtdIns and PtdInsP(2) in the mouse. PLoS One. 2013; 8:e58425; Lee H C, Inoue T, Imae R, Kono N, Shirae S, Matsuda S, et al. Caenorhabditis elegans mboa-7, a member of the MBOAT family, is required for selective incorporation of polyunsaturated fatty acids into phosphatidylinositol. Mol Biol Cell. 2008; 19: 1174-84), insights from a prior lipidomics study (Tanaka Y, Shimanaka Y, Caddeo A, Kubo T, Mao Y, Kubota T, et al. LPIAT1/MBOAT7 depletion increases triglyceride synthesis fueled by high phosphatidylinositol turnover. Gut. 2021; 70: 180-93), and the role of cholesterol trafficking-induced TAZ upregulation in MASH fibrosis. (Wang X, Cai B, Yang X, Sonubi O O, Zheng Z, Ramakrishnan R, et al. Cholesterol stabilizes TAZ in hepatocytes to promote experimental non-alcoholic steatohepatitis. Cell Metab. 2020; 31: 969-986.e967) The data support a pathway in which MBOAT7 LoF, by altering cellular PLs and increasing PSS1-mediated PS, activates cholesterol trafficking in hepatocytes (FIG. 14G). This pathway increases TAZ and its gene target, IHH, which is an HSC activator that promotes MASH fibrosis. (Wang X, Zheng Z, Caviglia J M, Corey K E, Herfel T M, Cai B, et al. Hepatocyte TAZ/WWTR1 promotes inflammation and fibrosis in nonalcoholic steatohepatitis. Cell Metab. 2016; 24: 848-62). Although cross-sectional in nature, human relevance is supported by the finding that the livers of individuals with rs641738-T had higher nuclear TAZ and IHH, whereas this association was not seen with MASH risk variants affecting PNPLA3 or HSD17B13. With these data in hand, future goals are to determine if the livers of individuals with rs641738-T show evidence of the altered PL pathway observed in MBOAT7-silenced hepatocytes and to conduct causation experiments in MASH mice based on this pathway, for example, to determine whether silencing hepatocyte TAZ or hepatocyte PSS1 mitigates the profibrotic effect of MBOAT7 LoF. Another future goal is to assess the role of MBOAT7 in a more advanced model of MASH fibrosis. In addition, given that rs641738-T is a risk factor for liver fibrosis in alcoholic-associated and hepatitis C-induced liver disease (Tanaka Y, Shimanaka Y, Caddeo A, Kubo T, Mao Y, Kubota T, et al. LPIAT1/MBOAT7 depletion increases triglyceride synthesis fueled by high phosphatidylinositol turnover. Gut. 2021; 70: 180-93; Thabet K, Asimakopoulos A, Shojaei M, Romero-Gomez M, Mangia A, Irving W L, et al. MBOAT7 rs641738 increases risk of liver inflammation and transition to fibrosis in chronic hepatitis C. Nat Commun. 2016; 7:12757), it will be interesting in the future to determine the relevance of this pathway to the overall process of injury-induced liver fibrosis.

There are undoubtedly additional mechanisms linking MBOAT7 LoF to MASH, possibly involving cell types. (Tanaka Y, Shimanaka Y, Caddeo A, Kubo T, Mao Y, Kubota T, et al. LPIAT1/MBOAT7 depletion increases triglyceride synthesis fueled by high phosphatidylinositol turnover. Gut. 2021; 70: 180-93; Helsley R N, Varadharajan V, Brown A L, Gromovsky A D, Schugar R C, Ramachandiran I, et al. Obesity-linked suppression of membrane-bound O-acyltransferase 7 (MBOAT7) drives nonalcoholic fatty liver disease. eLife. 2019; 8:e49882; Alharthi J, Bayoumi A, Thabet K, Pan Z, Gloss B S, Latchoumanin O, et al. A metabolic associated fatty liver disease risk variant in MBOAT7 regulates toll like receptor induced outcomes. Nat Commun. 2022; 13:7430) MBOAT7 deficiency in macrophages is associated with proinflammatory processes in patients with MAFLD and COVID-19. (Alharthi J, Bayoumi A, Thabet K, Pan Z, Gloss B S, Latchoumanin O, et al. A metabolic associated fatty liver disease risk variant in MBOAT7 regulates toll like receptor induced outcomes. Nat Commun. 2022; 13:7430). It is also possible that MBOAT7 deficiency in HSCs promotes their activation, as HSCs have high expression of MBOAT7 (Freund C, Wahlers A, Begli N H, Leopold Y, Klöters-Plachky P, Mehrabi A, et al. The MBOAT7 rs641738 variant is associated with an improved outcome in primary sclerosing cholangitis. Clin Res Hepatol Gastroenterol. 2020; 44:646-52). Moreover, certain types of LPI lipids can acutely induce hepatic inflammatory and fibrotic gene expression. (Helsley R N, Varadharajan V, Brown A L, Gromovsky A D, Schugar R C, Ramachandiran I, et al. Obesity-linked suppression of membrane-bound O-acyltransferase 7 (MBOAT7) drives nonalcoholic fatty liver disease. eLife. 2019; 8:e49882). Future studies will be required to understand how different effects of MBOAT7 LoF on various liver cell types are integrated to promote MASH and liver fibrosis.

Research into genetic risk factors for MASH has not yet led to new treatments for MASH fibrosis, although this is an active area of research. Given the high prevalence of rs641738-T and the fact that MBOAT7 is naturally decreased in the setting of obesity (Helsley R N, Varadharajan V, Brown A L, Gromovsky A D, Schugar R C, Ramachandiran I, et al. Obesity-linked suppression of membrane-bound O-acyltransferase 7 (MBOAT7) drives nonalcoholic fatty liver disease. eLife. 2019; 8:e49882; Helsley R N, Varadharajan V, Brown A L, Gromovsky A D, Schugar R C, Ramachandiran I, et al. Obesity-linked suppression of membrane-bound O-acyltransferase 7 (MBOAT7) drives nonalcoholic fatty liver disease. eLife. 2019; 8:e49882), therapeutic insights gained from studying this variant have great potential. In theory, new developments in mRNA therapeutics could lead to a therapy that restores MBOAT7 in individuals with rs641738-T. However, the level of expression needed to confer benefit may be challenging to achieve. While MBOAT7 expression must be high enough to restore normal PL dynamics, pilot dose testing of AAV8-TBG-Mboat7 suggested that excessive overexpression may not have a benefit. This latter finding, which may be caused by excessive PL remodeling, may help explain why a previous study using marked overexpression of hepatocyte MBOAT7 in MASH mice did not show a decrease in liver fibrosis. (Sharpe M C, Pyles K D, Hallcox T, Kamm D R, Piechowski M, Fisk B, et al. Enhancing hepatic MBOAT7 expression in mice with nonalcoholic steatohepatitis. Gastro Hep Adv. 2023; 2:558-72) Rather, described herein is another approach based on the new findings herein, together with previous work supporting the relevance of the cholesterol-TAZ-IHH pathway to human MASH and the druggability of this pathway using hepatocyte-targeted siTAZ (GalNAc-siTAZ) (Wang X, Zheng Z, Caviglia J M, Corey K E, Herfel T M, Cai B, et al. Hepatocyte TAZ/WWTR1 promotes inflammation and fibrosis in nonalcoholic steatohepatitis. Cell Metab. 2016; 24: 848-62; Wang X, Sommerfeld M R, Jahn-Hofmann K, Cai B, Filliol A, Remotti H E, et al. A therapeutic silencing RNA targeting Hepatocyte TAZ prevents and reverses fibrosis in nonalcoholic steatohepatitis in mice. Hepatol Commun. 2019; 3:1221-34; Wang X, Cai B, Yang X, Sonubi O O, Zheng Z, Ramakrishnan R, et al. Cholesterol stabilizes TAZ in hepatocytes to promote experimental non-alcoholic steatohepatitis. Cell Metab. 2020; 31: 969-986.e967; Wang X, Moore M P, Shi H, Miyata Y, Donnelly S K, Radiloff D R, et al. Hepatocyte-targeted siTAZ therapy lowers liver fibrosis in NASH diet-fed chimeric mice with hepatocyte-humanized livers. Mol Ther Methods Clin Dev. 2023; 31:101165), including in mice reconstituted with human hepatocytes. (Wang X, Moore M P, Shi H, Miyata Y, Donnelly S K, Radiloff D R, et al. Hepatocyte-targeted siTAZ therapy lowers liver fibrosis in NASH diet-fed chimeric mice with hepatocyte-humanized livers. Mol Ther Methods Clin Dev. 2023; 31:101165). Accordingly, treatment with GalNAc-siTAZ could be considered as a personalized medicine approach for individuals heterozygous or homozygous for the MBOAT7 rs641738 C>T who have other MASH risk factors and perhaps elevated plasma IHH, which is a marker of the TAZ/IHH pathway in humans with MASH (Moore M P, Wang X, Shi H, Meroni M, Cherubini A, Ronzoni L, et al. Circulating Indian hedgehog is a marker of the hepatocyte-TAZ pathway in experimental NASH and is elevated in humans with NASH. JHEP Rep. 2023; 5:100716). Moreover, future in vivo causation studies may suggest other targets in hepatocytes that are amenable to GalNAcsiRNA therapy, such as PSS1. Finally, as rs641738 C>T is a risk factor for liver fibrosis in other types of liver disease (Buch S, Stickel F, Trepo E, Way M, Herrmann A, Nischalke H D, et al. A genome-wide association study confirms PNPLA3 and identifies TM6SF2 and MBOAT7 as risk loci for alcohol-related cirrhosis. Nat Genet. 2015; 47:1443-8; Thabet K, Asimakopoulos A, Shojaei M, Romero-Gomez M, Mangia A, Irving W L, et al. MBOAT7 rs641738 increases risk of liver inflammation and transition to fibrosis in chronic hepatitis C. Nat Commun. 2016; 7:12757), these types of therapy may extend beyond MASH.

### Supplemental Materials and Methods

### AAV8 Viruses

Mice were fed the FPC diet for 8 weeks to induce hepatosteatosis (Wang X, Zheng Z, Caviglia J M, Corey K E, Herfel T M, Cai B, Masia R, et al. Hepatocyte TAZ/WWTR1 promotes inflammation and fibrosis in nonalcoholic steatohepatitis. Cell Metab 2016; 24:848-862) and then tail vein-injected with adeno-associated virus subtype 8 (AAV8)-TBG-Mboat7 (1.5×1011 genome copies/mouse) or AAV8-H1-shMboat7 or control AAV8-H1-shCtrl. AAV8-TBG-Mboat7 (AAV-264332) was from Vector Biolabs. AAV8-TBG-GFP (#105535), was purchased from the Addgene. Wildtype mouse Mboat7 plasmid was from Origene (#MR207548). AAV8-H1-shMboat7 was made by annealing complementary oligonucleotides (SEQ ID NO: 10-5′CACCAtgatggagacactcagctataTCAAGAGTATAGCTGAGTGTCTCCATCA3′), which were then ligated into the self-complementary (sc) AAV8-RSV-GFP-H1 vector as described previously. (Wang X, Zheng Z, Caviglia J M, Corey K E, Herfel T M, Cai B, Masia R, et al. Hepatocyte TAZ/WWTR1 promotes inflammation and fibrosis in nonalcoholic steatohepatitis. Cell Metab 2016; 24:848-862). The resultant constructs were amplified by Vector Biolabs, Malvern, PA.

siRNA-Mediated Silencing, CRISPR-Cas9-Mediated Knockdown, or Transfection of Genes in Hepatocytes

Scrambled siRNA control and oligo-targeting siRNAs were transfected into AML12 or primary human or mouse hepatocytes using Lipofectamine RNAiMAX (Life Technologies) at 40 nM of siRNA in 24-well plates following the manufacturer's instructions. Briefly, 2×105 cells at 50-70% confluence were cultured overnight and then incubated for 6 h in 0.5 ml of medium containing 1.5 μl Lipofectamine RNAiMAX and 20 pmol siRNA. Cells were harvested 48 h following transfection after the addition of Laemmli Sample Buffer (Bio-Rad, #1610737) containing 2-mercaptoethanol (Bio-Rad, #161-0710) for immunoblotting. The siRNA sequences are listed in FIG. 22. Murine Mboat7 plasmid was transfected into AML12 cells using Lipofectamine® LTX Reagent with PLUS™ Reagent (Life Technologies, #15338100). For each well in a 24-well plate, 2 μl LTX, 0.5 μl PLUS reagent, and 0.5 μg plasmid DNA was used when cells reached 30-40% confluence. The cells were harvested after overnight incubation. AML12 hepatocytes lacking AGPAT1 and PSS1, encoded by Ptdss1, were generated by CRISPR-Cas9. (Ran F A, Hsu P D, Wright J, Agarwala V, Scott D A, Zhang F. Genome engineering using the CRISPR-Cas9 system. Nat Protoc 2013; 8:2281-2308). Guide RNA sequences against Agpat1 and Ptdss1 are as follows: Agpat1 gRNA (SEQ ID No: 11): /A1tR1/rArCrArArCrCrArCrGrUrArGrGrGrCrUrGrCrGrUrGrUrUrUrUrArGrArGrCrUrArU rGrCrU/AltR2/and Ptdss1 gRNA (SEQ ID No: 12): /A1tR1/rUrCrGrArUrArUrGrCrUrArCrArCrGrArGrArArGrGrUrUrUrUrArGrArGrCrUrArU rGrCrU/AltR2/. The cells for these experiments were cultured in DMEM containing 10% FBS, with the exception of the AML12 experiments in which MBOAT7 was silenced together with the silencing or CRISPR-Cas9-mediated knock down of other lipid gene. For these experiments, the cells were cultured in DMEM containing either 0.1% FBS or 1% FBS based on pilot experiments examining the best growth conditions for the cells, which varied depending on which lipid-related genes were silenced or knocked down. Medium with 0.10% FBS was used for experiments involving siMboat7 plus siAsterB/C or siCds2, while medium with 1% FBS was used for experiments involving siMboat7 plus knockdown of AGPAT1 or PSS1.

Colorimetric and ELISA assays

Fasting blood glucose was measured using a glucose meter (One Touch Ultra, LifeScan) in mice that were fasted for 5 h, with free access to water. Plasma ALT concentration was measured following kit instruction. Active RhoA was assayed in AML12 hepatocytes using an ELISA assay according to manufacturer's instructions (G-LISA®, #BK124, Cytoskeleton).

### Immunohistochemical Analysis

Livers were fixed in 10% formalin for at least 24 h before paraffin embedding and then sectioned for histological analysis, including hematoxylin and eosin (H&E) staining and Picrosirius Red staining (Polysciences, #24901), according to the manufacturer's protocol. Liver paraffin sections were deparaffinized with xylene, hydrated, and then subjected to antigen retrieval using citrate sodium (Vector laboratory, #H-3300, 1:100 dilution) in a high-pressure cooker for 10 min. Next, the liver sections were incubated with 3% hydrogen peroxide (Sigma, #H1009) for 10 min at room temperature to block endogenous peroxidase activity, blocked with 5% donkey serum in PBS with 0.1% Triton X-100 (Sigma, #X100) for 1 h at room temperature, and incubated at 4° C. overnight with anti-mouse collagen 1a1 (Col1a1, Cell signaling technology, #72026S, RRID:AB_2904565, 1:200 dilution), osteopontin (Opn, RD, #AF808, RRID:AB_2194992, 1:1000 dilution), cytokeratin 19 (CK19, DSHB, TROMA-III, RRID:AB_2133570, 1:500 dilution), F4/80 (Cell signaling technology, #70076s, RRID:AB_2799771, 1:200), IL-1β (R&D Systems, AF-401-SP), IL6 (R&D Systems, #AF-406), and TNFα (R&D Systems, #AF-410-NA) in PBS containing 1% donkey serum. The sections were then incubated with SignalStain® Boost IHC detection reagent from Cell Signaling Technology (HRP, Rabbit, #8114; HRP, Rat, #72838; HRP, Goat, #63707), followed by color development using a DAB substrate kit (Cell signaling technology, #8059). The sections were counterstained with hematoxylin, dehydrated, and mounted. The images were captured using a Nikon microscope, and the data were analyzed using Image J software. For all analyses, 6 randomly chosen fields were quantified per section per mouse. To assess nuclear TAZ in human liver sections, paraffin sections were rehydrated and subjected to antigen retrieval by placing in a pressure cooker for 10 mins in Target Retrieval Solution (Dako, S1699). The slides were then treated with 3% hydrogen peroxide for 10 min and then blocked with Serum-Free Protein Block (Dako, X0909) for 30 min. Sections were incubated overnight with TAZ primary antibody (Millipore Sigma, HPA007415) and then developed with DAB substrate kit (Cell Signaling, #8059). Images were captured randomly, and quantification was conducted without knowledge of cohort assignment. This work was performed in the Molecular Pathology Shared Resource of the Herbert Irving Comprehensive Cancer Center at Columbia University, supported by NIH grant #P30 CA013696 (National Cancer Institute).

### Liver Immunofluorescence Staining

For αSMA immunofluorescent staining, liver paraffin sections were deparaffinized, hydrated, subject to antigen retrieval using citrate sodium (Vector laboratory, #H-3300, 1:100 dilution) in a high-pressure cooker for 10 min. For MBOAT7 immunofluorescent staining, frozen liver sections were thawed for 30 minutes at room temperature and then subjected to antigen retrieval using (Vector laboratory, #H-3300) in a warm-bath at 70° C. for 25 minutes. Liver sections were blocked with 5% donkey serum in PBS with 0.1% Triton X-100 for 1 h at room temperature and then incubated at 4° C. overnight with anti-α-smooth muscle actin (Sigma, #C6198, RRID:AB_476856, 1:100 dilution), MBOAT7 (Custom made monoclonal (FT10), PMID: 23097495, 1:100 dilution), His (27E8, Cell Signaling, 2366S), and 4-hydroxynonenal (4-HNE; Millipore AB5605) in PBS containing 1% donkey serum. The sections were incubated with fluorescent dye-conjugated secondary antibodies (1:250 dilution) for 1 h at room temperature, followed by nuclei staining with DAPI. The images were captured using a Leica DMI 6000B fluorescence microscope. For all analyses, six randomly chosen fields were quantified per section per mouse and the data were analyzed using Image J software.

### Immunoblotting

Liver protein was extracted using RIPA lysis buffer (Thermo, #89901) with a proteinase and phosphatase inhibitor cocktail (Thermo, #78445), followed by protein concentration measurement using a BCA kit (Thermo, #23227). Cultured cells were lysed in RIPA buffer or Laemmli sample buffer (Bio-Rad, #161-0737) containing 5% 2-mercaptoethanol, heated at 100° C. for 5 min. 10-20 μg total protein was separated on 4-20% Tris gels (Life technologies, EC60285) and transferred to nitrocellulose membranes (Bio-Rad, #1620115). The membranes were blocked with 5% nonfat milk in Tris-buffered saline with 0.1% Tween 20 (TBST) for 1 h at room temperature, The membranes were then incubated overnight at 4° C. with antibodies recognizing TAZ (Cell Signaling #8418), IHH (Proteintech #13388-1-AP), MBOAT7 (Custom made monoclonal FT10 (Lee H C, Inoue T, Sasaki J, Kubo T, Matsuda S, Nakasaki Y, Hattori M, et al. LPIAT1 regulates arachidonic acid content in phosphatidylinositol and is required for cortical lamination in mice. Mol Biol Cell 2012; 23:4689-4700), CDS2 (Novous Biologicals, NBP1-86435), p-Ser89-Taz (Invitrogen, PA5-105066), SREBP1 (Novous Biologicals, NB600-582SS), SREBP2 (Novous Biologicals, NB100-74543), p-S133-CREB (Cell signaling, 9198), CREB (Cell signaling, 9197), Calnexin (Abcam, ab10286), and His (27E8, Cell Signaling, 2366S). The membranes were then incubated with HRP-conjugated secondary antibodies (Jackson ImmunoResearch, Peroxidase AffiniPure Donkey anti-rat IgG RRID:AB_2340639 and RRID:AB_10015282; Cell Signaling anti-rabbit IgG, HRP-linked antibody #7074; 1:5000 dilution) for 1 h at room temperature, and bands were detected with SuperSignal West Pico PLUS Chemiluminescent Substrate (Thermo, #34580). Image ImageJ was used to measure intensities of bands and relative to R-Actin (Cell Signaling #5125) or GAPDH (Cell Signaling #3683).

### Quantitative RT-qPCR

Total RNA was extracted from liver tissue, cultured hepatocytes, or liver spheroids using the RNeasy kit (Qiagen, 74106). The quality and concentration of the RNA was assessed by absorbance at 260 and 280 nm using a Thermo Scientific NanoDrop spectrophotometer. cDNA was synthesized from 1 μg total RNA using oligo (dT) and Superscript II (Invitrogen). qPCR was performed with a 7500 Real time PCR system (Applied Biosystems) using SYBR Green Master Mix (Life Technologies, #4367659). The primer sequences are listed in FIG. 23.

Liquid Chromatography with Tandem Mass Spectrometry (LC-MS-MS)

AML12 cells (3×106 cells) were treated with siCtrl or siMboat7 as described above. The media were aspirated, and the cells were scrapped with water, followed by centrifugation at 200 g for 5 min at 4° C. The pellet was resuspended in 1 ml cold PBS and centrifuged at 200 g for 5 min at 4° C. The PBS was aspirated, and the cell pellets were stored at −20° C. until processing. Liver tissue from mice was harvested at the time of euthanasia, snap frozen in liquid nitrogen, and stored at −80° C. until processing. Targeted lipidomic analysis by LC-MS/MS was carried out by the Biomarkers Core Laboratory at Columbia University Irving Medical Center as previously described. (Chen J, Cazenave-Gassiot A, Xu Y, Piroli P, Hwang R, Jr., DeFreitas L, Chan R B, et al. Lysosomal phospholipase A2 contributes to the biosynthesis of the atypical late endosome lipid bis(monoacylglycero)phosphate. Commun Biol 2023; 6:210; Miltenberger-Miltenyi G, Jones A, Tetlow A M, Conceição V A, Crary J F, Ditzel R M, Jr., Farrell K, et al. Sphingolipid and phospholipid levels are altered in human brain in chorea-acanthocytosis. Mov Disord 2023; 38:1535-1541; Chen J, Soni R K, Xu Y, Simoes S, Liang F X, DeFreitas L, Hwang R, Jr., et al. Juvenile CLN3 disease is a lysosomal cholesterol storage disorder: similarities with Niemann-Pick type C disease. EBioMedicine 2023; 92:104628) Briefly, lipid extracts were prepared from tissue homogenates spiked with appropriate internal standards using a modified Bligh and Dyer method and analyzed on a platform comprising Agilent 1260 Infinity HPLC integrated to Agilent 6490 QQQ mass spectrometer controlled by Masshunter v7.0 (Agilent Technologies). Quantification of lipid species was accomplished using both normal phase and reverse phase chromatography under multiple reaction monitoring (MRM), positive and negative ionization modes in conjunction with referencing of appropriate internal standards: PA 14:0/14:0, PC 14:0/14:0, PE 14:0/14:0, PG 15:0/15:0, PI 17:0/20:4, PS 14:0/14:0, LPI 13:0, D7-cholesterol, CE 17:0, 4ME 16:0 diether DG, D5-TG 16:0/18:0/16:0 (Avanti Polar Lipids, Alabaster, AL). Lipid levels for each sample were calculated by summing the total number of moles of all lipid species measured by all three LCMS methodologies and then normalizing the total to mol %. The final data are presented as mean nmol/mg tissue or nmol/well.

### Assessing HSC Activation Ex Vivo Through Hepatocyte-to-HSC Conditioned Medium Transfer

For conditioned medium transfer, hepatocytes isolated from Mboat7fl/fl mice injected with AAV8-TBG-Cre (to delete hepatocyte MBOAT7) or AAV8-TBG-GFP were incubated for 6 h with 0.4 mM oleic acid. The cells were cultured for an additional 48 h in DMEM/10% FBS, after which the conditioned media were collected and added to primary mouse HSCs±the Smoothened inhibitor cyclopamine (3 M). After the 48 h, the HSCs were assayed by qPCR for the expression of genes associated with HSC activation.

### Assessing HSC Activation Ex Vivo Through Human Liver Spheroids

For liver spheroids, primary human hepatocytes and non-parenchymal cells were co-incubated to form spheroids as described. (Hurrell T, Kastrinou-Lampou V, Fardellas A, Hendriks D F G, Nordling Å, Johansson I, Baze A, et al. Human liver spheroids as a model to study aetiology and treatment of hepatic fibrosis. Cells 2020; 9). In brief, fresh hepatocytes and non-parenchymal cells obtained from patients undergoing liver resection at the University of Pittsburgh were seeded into 96-well Akura™ Spheroid Microplates (inSphero, Schlieren, Switzerland) at a ratio of 4:1 (hepatocytes:non-parenchymal cells; 2000 viable cells per well) in 70 μL of William's E medium supplemented with 2 mM L-glutamine (Thermo Fisher), 1× penicillin-streptomycin solution (Corning, #30-002-Cl), 100 nM dexamethasone (Thermo Fisher), ITS X-100 (Thermo Fisher), and 10% fetal bovine serum (FBS, Thermo Fisher) under standard cell culture conditions at 37° C. in a humidified incubator with 5% CO2. On day 5 after seeding, and every 2-3 days thereafter, the spheroids were refreshed with the medium described above but minus FBS. The spheroids were treated with siMBOAT7 or scrambled siRNA on day 7 and harvested on day 10 after seeding.

As will be apparent to one of ordinary skill in the art from a reading of this disclosure, the present disclosure can be embodied in forms other than those specifically disclosed above. The particular embodiments described above are, therefore, to be considered as illustrative and not restrictive. Those skilled in the art will recognize, or be able to ascertain, using no more than routine experimentation, numerous equivalents to the specific embodiments described herein.

The scope of the invention is as set forth in the appended claims and equivalents thereof, rather than being limited to the examples contained in the foregoing description. The contents of all of the references disclosed herein are incorporated by reference in their entirety.

### REFERENCES

Wang X, Zheng Z, Caviglia J M, Corey K E, Herfel T M, Cai B, Masia R, et al. Hepatocyte TAZ/WWTR1 promotes inflammation and fibrosis in nonalcoholic steatohepatitis. Cell Metab 2016; 24:848-862.
Ran F A, Hsu P D, Wright J, Agarwala V, Scott D A, Zhang F. Genome engineering using the CRISPR-Cas9 system. Nat Protoc 2013; 8:2281-2308.
Lee H C, Inoue T, Sasaki J, Kubo T, Matsuda S, Nakasaki Y, Hattori M, et al. LPIAT1 regulates arachidonic acid content in phosphatidylinositol and is required for cortical lamination in mice. Mol Biol Cell 2012; 23:4689-4700.
Chen J, Cazenave-Gassiot A, Xu Y, Piroli P, Hwang R, Jr., DeFreitas L, Chan R B, et al. Lysosomal phospholipase A2 contributes to the biosynthesis of the atypical late endosome lipid bis(monoacylglycero)phosphate. Commun Biol 2023; 6:210.
Miltenberger-Miltenyi G, Jones A, Tetlow A M, Conceição V A, Crary J F, Ditzel R M, Jr., Farrell K, et al. Sphingolipid and phospholipid levels are altered in human brain in chorea-acanthocytosis. Mov Disord 2023; 38:1535-1541.
Chen J, Soni R K, Xu Y, Simoes S, Liang F X, DeFreitas L, Hwang R, Jr., et al. Juvenile CLN3 disease is a lysosomal cholesterol storage disorder: similarities with Niemann-Pick type C disease. EBioMedicine 2023; 92:104628.
Hurrell T, Kastrinou-Lampou V, Fardellas A, Hendriks D F G, Nordling Å, Johansson I, Baze A, et al. Human liver spheroids as a model to study aetiology and treatment of hepatic fibrosis. Cells 2020; 9.
Younossi Z M, Golabi P, Paik J M, Henry A, Van Dongen C, Henry L. The global epidemiology of nonalcoholic fatty liver disease (NAFLD) and nonalcoholic steatohepatitis (NASH): A systematic review. Hepatology. 2023; 77:1335-47.
Angulo P, Kleiner D E, Dam-Larsen S, Adams L A, Bjomsson E S, Charatcharoenwitthaya P, et al. Liver fibrosis, but no other histologic features, is associated with long-term outcomes of patients with nonalcoholic fatty liver disease. Gastroenterology. 2015; 149:389-397.e310.
Wree A, Broderick L, Canbay A, Hoffman H M, Feldstein A E. From NAFLD to NASH to cirrhosis-new insights into disease mechanisms. Nat Rev Gastroenterol Hepatol. 2013; 10: 627-36.
Bohinc B N, Diehl A M. Mechanisms of disease progression in NASH: New paradigms. Clin Liver Dis. 2012; 16:549-65.
Ray K. Resmetirom proves positive for NASH with liver fibrosis. Nat Rev Gastroenterol Hepatol. 2024; 21:218.
Busca C, Arias P, Sánchez-Conde M, Rico M, Montejano R, Martin-Carbonero L, et al. Genetic variants associated with steatohepatitis and liver fibrosis in HIV-infected patients with NAFLD. Front Pharmacol. 2022; 13:905126.
Dongiovanni P, M Anstee Q, Valenti L. Genetic predisposition in NAFLD and NASH: Impact on severity of liver disease and response to treatment. Curr Pharmaceut Des. 2013; 19:5219-38.
Krawczyk M, Rau M, Schattenberg J M, Bantel H, Pathil A, Demir M, et al. Combined effects of the PNPLA3 rs738409, TM6SF2rs58542926, and MBOAT7 rs641738 variants on NAFLD severity: A multicenter biopsy-based study. J Lipid Res. 2017; 58:247-55.
Luukkonen P K, Zhou Y, Hyötyläinen T, Leivonen M, Arola J, Orho-Melander M, et al. The MBOAT7 variant rs641738 alters hepatic phosphatidylinositols and increases severity of nonalcoholic fatty liver disease in humans. J Hepatol. 2016; 65:1263-5.
Mancina R M, Dongiovanni P, Petta S, Pingitore P, Meroni M, Rametta R, et al. The MBOAT7-TMC4 variant rs641738 increases risk of nonalcoholic fatty liver disease in individuals of European descent. Gastroenterology. 2016; 150:1219-230.e1216.
Meroni M, Longo M, Fracanzani A L, Dongiovanni P. MBOAT7 down-regulation by genetic and environmental factors predisposes to MAFLD. EBioMedicine. 2020; 57:102866.
Pazoki R, Vujkovic M, Elliott J, Evangelou E, Gill D, Ghanbari M, et al. Genetic analysis in European ancestry individuals identifies 517 loci associated with liver enzymes. Nat Commun. 2021; 12:2579.
Raja A M, Ciociola E, Ahmad I N, Dar F S, Naqvi S M S, Moaeen-Ud-Din M, et al. Genetic susceptibility to chronic liver disease in individuals from Pakistan. Int J Mol Sci. 2020
Teo K, Abeysekera K W M, Adams L, Aigner E, Anstee Q M, Banales J M, et al. rs641738C>T near MBOAT7 is associated with liver fat, ALT and fibrosis in NAFLD: A meta-analysis. J Hepatol. 2021; 74:20-30.
Thangapandi V R, Knittelfelder O, Brosch M, Patsenker E, Vvedenskaya O, Buch S, et al. Loss of hepatic Mboat7 leads to liver fibrosis. Gut. 2021; 70:940-50.
Xu X, Xu H, Liu X, Zhang S, Cao Z, Qiu L, et al. MBOAT7 rs641738 (C>T) is associated with NAFLD progression in men and decreased ASCVD risk in elder Chinese population. Front Endocrinol (Lausanne). 2023; 14:1199429.
Chen Y, Du X, Kuppa A, Feitosa M F, Bielak L F, O'Connell J R, et al. Genome-wide association meta-analysis identifies 17 loci associated with nonalcoholic fatty liver disease. Nat Genet. 2023; 55:1640-50.
Anderson K E, Kielkowska A, Durrant T N, Juvin V, Clark J, Stephens L R, et al. Lysophosphatidylinositol-acyltransferase-1 (LPIAT1) is required to maintain physiological levels of PtdIns and PtdInsP(2) in the mouse. PLoS One. 2013; 8:e58425.
Lee H C, Inoue T, Imae R, Kono N, Shirae S, Matsuda S, et al. Caenorhabditis elegans mboa-7, a member of the MBOAT family, is required for selective incorporation of polyunsaturated fatty acids into phosphatidylinositol. Mol Biol Cell. 2008; 19:1174-84.
Buch S, Stickel F, Trepo E, Way M, Herrmann A, Nischalke H D, et al. A genome-wide association study confirms PNPLA3 and identifies TM6SF2 and MBOAT7 as risk loci for alcohol-related cirrhosis. Nat Genet. 2015; 47:1443-8.
Helsley R N, Varadharajan V, Brown A L, Gromovsky A D, Schugar R C, Ramachandiran I, et al. Obesity-linked suppression of membrane-bound O-acyltransferase 7 (MBOAT7) drives nonalcoholic fatty liver disease. eLife. 2019; 8:e49882.
Tanaka Y, Shimanaka Y, Caddeo A, Kubo T, Mao Y, Kubota T, et al. LPIAT1/MBOAT7 depletion increases triglyceride synthesis fueled by high phosphatidylinositol turnover. Gut. 2021; 70:180-93.
Varadharajan V, Massey W J, Brown J M. Membrane-bound O-acyltransferase 7 (MBOAT7)-driven phosphatidylinositol remodeling in advanced liver disease. J Lipid Res. 2022; 63:100234.
Xia M, Chandrasekaran P, Rong S, Fu X, Mitsche M A. Hepatic deletion of Mboat7 (LPIAT1) causes activation of SREBP-1c and fatty liver. J Lipid Res. 2021; 62:100031.
Sharpe M C, Pyles K D, Hallcox T, Kamm D R, Piechowski M, Fisk B, et al. Enhancing hepatic MBOAT7 expression in mice with nonalcoholic steatohepatitis. Gastro Hep Adv. 2023; 2:558-72.
Wang X, Zheng Z, Caviglia J M, Corey K E, Herfel T M, Cai B, et al. Hepatocyte TAZ/WWTR1 promotes inflammation and fibrosis in nonalcoholic steatohepatitis. Cell Metab. 2016; 24:848-62.
Wang X, Sommerfeld M R, Jahn-Hofmann K, Cai B, Filliol A, Remotti H E, et al. A therapeutic silencing RNA targeting Hepatocyte TAZ prevents and reverses fibrosis in nonalcoholic steatohepatitis in mice. Hepatol Commun. 2019; 3:1221-34.
Wang X, Cai B, Yang X, Sonubi O O, Zheng Z, Ramakrishnan R, et al. Cholesterol stabilizes TAZ in hepatocytes to promote experimental non-alcoholic steatohepatitis. Cell Metab. 2020; 31:969-986.e967.
Wang X, Moore M P, Shi H, Miyata Y, Donnelly S K, Radiloff D R, et al. Hepatocyte-targeted siTAZ therapy lowers liver fibrosis in NASH diet-fed chimeric mice with hepatocyte-humanized livers. Mol Ther Methods Clin Dev. 2023; 31:101165.
Neuschwander-Tetri B A, Loomba R, Sanyal A J, Lavine J E, Van Natta M L, Abdelmalek M F, et al. Farnesoid X nuclear receptor ligand obeticholic acid for non-cirrhotic, non-alcoholic steatohepatitis (FLINT): A multicentre, randomised, placebo-controlled trial. Lancet. 2015; 385:956-65.
Baselli G A, Dongiovanni P, Rametta R, Meroni M, Pelusi S, Maggioni M, et al. Liver transcriptomics highlights interleukin-32 as novel NAFLD-related cytokine and candidate biomarker. Gut. 2020; 69:1855-66.
Cherubini A, Ostadreza M, Jamialahmadi O, Pelusi S, Rrapaj E, Casirati E, et al. Interaction between estrogen receptor-α and PNPLA3 p. I148M variant drives fatty liver disease susceptibility in women. Nat Med. 2023; 29:2643-55.
Ozcan L, Wong C C, Li G, Xu T, Pajvani U, Park S K, et al. Calcium signaling through CaMKII regulates hepatic glucose production in fasting and obesity. Cell Metab. 2012; 15:739-51.
Johnson K A, Radhakrishnan A. The use of anthrolysin O and ostreolysin A to study cholesterol in cell membranes. Methods Enzymol. 2021; 649:543-66.
Ferrari A, He C, Kennelly J P, Sandhu J, Xiao X, Chi X, et al. Aster proteins regulate the accessible cholesterol pool in the plasma membrane. Mol Cell Biol. 2020; 40:e00255-20
He C, Hu X, Weston T A, Jung R S, Sandhu J, Huang S, et al. Macrophages release plasma membrane-derived particles rich in accessible cholesterol. Proc Natl Acad Sci USA. 2018; 115:E8499-508.
Xiao X, Kennelly J P, Ferrari A, Clifford B L, Whang E, Gao Y, et al. Hepatic nonvesicular cholesterol transport is critical for systemic lipid homeostasis. Nat Metab. 2023; 5:165-81.
Lee S J, Park J B, Kim K H, Lee W R, Kim J Y, An H J, et al. Immunohistochemical study for the origin of ductular reaction in chronic liver disease. Int J Clin Exp Pathol. 2014; 7:4076-85.
Zhao L, Westerhoff M, Pai R K, Choi W T, Gao Z H, Hart J. Centrilobular ductular reaction correlates with fibrosis stage and fibrosis progression in non-alcoholic steatohepatitis. Mod Pathol. 2018; 31:150-9.
Dongiovanni P, Romeo S, Valenti L. Genetic factors in the pathogenesis of nonalcoholic fatty liver and steatohepatitis. Biomed Res Int. 2015; 2015:460190.
Lagace T A. Phosphatidylcholine: Greasing the cholesterol transport machinery. Lipid Insights. 2015; 8:65-73.
Wang B, Tontonoz P. Phospholipid remodeling in physiology and disease. Annu Rev Physiol. 2019; 81:165-88.
Sandhu J, Li S, Fairall L, Pfisterer S G, Gurnett J E, Xiao X, et al. Aster proteins facilitate nonvesicular plasma membrane to E R cholesterol transport in mammalian cells. Cell. 2018; 175:514-529.e520.
Lange Y, Ye J, Steck T L. How cholesterol homeostasis is regulated by plasma membrane cholesterol in excess of phospholipids. Proc Natl Acad Sci USA. 2004; 101:11664-7.
Tabas I, Rosoff W J, Boykow G C. Acyl coenzyme A:cholesterol acyl transferase in macrophages utilizes a cellular pool of cholesterol oxidase-accessible cholesterol as substrate. J Biol Chem. 1988; 263:1266-72.
Trinh M N, Brown M S, Seemann J, Vale G, McDonald J G, Goldstein J L, et al. Interplay between Asters/GRAMD1s and phosphatidylserine in intermembrane transport of LDL cholesterol. Proc Natl Acad Sci. 2022; 119:e2120411119.
Sturbois-Balcerzak B, Stone S J, Sreenivas A, Vance J E. Structure and expression of the murine phosphatidylserine synthase-1 gene. J Biol Chem. 2001; 276:8205-12.
Kennedy E P, Weiss S B. The function of cytidine coenzymes in the biosynthesis of phospholipides. J Biol Chem. 1956; 222:193-214.
Thabet K, Asimakopoulos A, Shojaei M, Romero-Gomez M, Mangia A, Irving W L, et al. MBOAT7 rs641738 increases risk of liver inflammation and transition to fibrosis in chronic hepatitis C. Nat Commun. 2016; 7:12757.
Alharthi J, Bayoumi A, Thabet K, Pan Z, Gloss B S, Latchoumanin 0, et al. A metabolic associated fatty liver disease risk variant in MBOAT7 regulates toll like receptor induced outcomes. Nat Commun. 2022; 13:7430.
Freund C, Wahlers A, Begli N H, Leopold Y, Kloters-Plachky P, Mehrabi A, et al. The MBOAT7 rs641738 variant is associated with an improved outcome in primary sclerosing cholangitis. Clin Res Hepatol Gastroenterol. 2020; 44:646-52.
Moore M P, Wang X, Shi H, Meroni M, Cherubini A, Ronzoni L, et al. Circulating Indian hedgehog is a marker of the hepatocyte-TAZ pathway in experimental NASH and is elevated in humans with NASH. JHEP Rep. 2023; 5:100716.

What is claimed is:

1. A method of treating or preventing metabolic dysfunction-associated steatohepatitis (MASH) in a subject in need thereof, the method comprising administering to the subject a composition targeting the hepatic TAZ pathway.

2. The method of claim 1, wherein the composition reduces or inhibits TAZ expression in the subject when compared to untreated subjects or to expression level of TAZ in the subject pre-treatment.

3. The method of claim 2, wherein the composition comprises a TAZ siRNA.

4. The method of claim 1, wherein the composition increases membrane bound O-acyltransferase domain containing 7 (MBOAT7) expression.

5. The method of claim 3, wherein the composition comprises MBOAT7 mRNA.

6. The method of claim 4, wherein the composition comprises either SEQ ID NO: 1 or 2.

7. The method of claim 4, wherein the composition comprises an mRNA encoding either SEQ ID NO: 3 or 4.

8. The method of claim 4, wherein the composition comprises an mRNA nanoparticle.

9. The method of claim 1, wherein the composition comprises a viral vector comprising a nucleic acid encoding MBOAT7.

10. The method of claim 8, wherein the viral vector is an adeno-associated vector (AAV).

11. The method of claim 9, wherein the viral vector is AAV8.

12. The method of claim 1, wherein the patient is a human.

13. The method of claim 11, wherein the human expresses the rs641738 variant of the MBOAT7 gene.

14. A composition for treating or preventing metabolic dysfunction-associated steatohepatitis (MASH), comprising an expression vector capable of targeting the hepatic TAZ pathway, wherein the composition increases MBOAT7 expression.

15. The composition of claim 13, wherein the composition comprises MBOAT7 mRNA.

16. The composition of claim 15, wherein the composition comprises either SEQ ID NO: 1 or 2.

17. The composition of claim 15, wherein the composition comprises an mRNA encoding either SEQ ID NO: 3 or 4.

18. The composition of claim 15, wherein the composition comprises an mRNA nanoparticle.

19. The composition of claim 15, wherein the composition is a viral vector.

20. The composition of claim 19, wherein the viral vector is an AAV vector.

21. The composition of claim 19, wherein the viral vector is AAV8.


SEQUENCE LISTING (70 sequences)

SEQ ID NO: 1 - DNA, 2341 bp, Mus sp.
  source 1..2341: mol_type = genomic DNA
agctctttgcccggggacctcctagaaagatcgcgacaggcatcttgcggcagactggtgttctgggtacctttcttgcggtgctgtaactcgtacagccgcggctctcggggcctggaccgcgcagccctgccggcgccctccagaacgggcagtgcgggggcgtgctgagctggggaggcgtggcgcgagccgaggcggcctcgaaaaggagctccgcagtcttctggcccacggacggttcagaccatgacacccgaagaatggacatatctaatggtccttcttatctccatccctgttggcttcctctttaagaaagctggacctgggctgaagagatggggggcagcagctgtgggcctggggctcaccttattcacctgtggcccccacagtttgcattctctgatcaccatcttgggaacctgggccctcattcaggcccagccctgctcctgccatgccctggctcttgcctggaccttctcctatctcctcttcttccgagccctcagcctgctgggcctgcccactcccacgcccttcaccaatgctgtccagctgctgttgacactgaagttggtgagtctagctagtgaagtccaggatctgcatctggctcagagaaaggaaatagcctccggcttccacaaggagcctacgctgggcctcctgcctgaggtcccctctttgatggagacactcagctatagctactgttacgtgggaatcatgacaggcccattcttccgctaccgcacctacctggattggctggaacagcccttcccggaagccgtgcccagcctgaggcccctgctgcgccgcgcctggccagccccgctctttggcctgctcttcctgctgtcctcccatctcttcccactggaagctgtgcgtgaggacgccttctacgcccgcccgctgcccacccgcctcttctacatgatcccggtcttcttcgccttccgcatgcgcttctacgttgcctggattgcggccgagtgcggttgcattgccgcgggcttcggggcctaccctgtggctgccaaagcccgggccgggggcggccccaccctccaatgcccaccccctagcagtccggagattgcagcttccctggagtatgactatgagaccatccgtaacatcgactgctatggcacagacttctgcgtgcgtgtgcgggatggcatgcgatactggaacatgaccgtgcagtggtggctggcacagtacatctacaagagcgcacctttccgctcctacgttttgaggagtgcctggaccatgctgttgagtgcctactggcatggcctccaccctggttactacctaagcttcatgaccatcccgctgtgcctggctgctgagggctatttggagtcagccttgcggagacacctgagccccgggggccagaaagcctgggactgggtccactggttcctgaagatgcgtgcctacgactacatgtgcatgggctttgtgctcctttccatggctgacacactccggtactgggcctccatctacttctgggtccactttctagccctggcttgcttggggctggggctggttttgggtgggggcagccccagcaagaggaagacaccatcccaggccaccagcagccaagcgaaggaaaagctccgggaagagtgagctctgctgcattggcctgccttccagttcaagcttttttgggaattccatgaaccaggctgtttgtttggggtttttgtttgtttgtttgtttgtttcctttacccagcaagaatcccttgtttggctaagagcctggagaggatcccctcttcccaaataattcctctgccttctatttaaagctaaggtatccttctcttgggctctctcagcatcttgaccttttcagaccttcctctgctaacatcagggtattactatccactcttgaacctattatctctgcaacaatcttcagatgttcaaaaagccacacttcccaaaaatgcccttgcagggaccagtggtcatctggcatcttagacagactcccagtgggtccccagtatgggggcaggaacttcagggccaggttctgggagaggggagggatagccttcttgttttcctctttgtttttatcatcacaccagtgtttcagagaccatggtcttacacatgctgaaggagaagctaaaatgtgagaagccccaggggagcttgctcttacagcagcttctgcctgagccatttctgggctccccatacaacctaccacccagtgtcatccttggcctgtgacaggccagaatgtataaagctttcccaataaagtgttacacatgca

SEQ ID NO: 2 - DNA, 2294 bp, Homo sapiens
  source 1..2294: mol_type = genomic DNA
agtgtggacctggactcgaatcccgttgccgactcgcgctctcggcttctgctccggggcttcttccctgcccgcccggggccctgaccgtggcttcttccccggcctgatctgcgcagcccggcgggcgcccagaaggagcaggcggcgcgggggcgcgctgggcgggggaggcgtggccggagctgcggcggcaagcgggctgggactgctcggccgcctcctgcccggcgagcagctcagaccatgtcgcctgaagaatggacgtatctagtggttcttcttatctccatccccatcggcttcctctttaagaaagccggtcctgggctgaagagatggggagcagccgctgtgggcctggggctcaccctgttcacctgtggcccccacactttgcattctctggtcaccatcctcgggacctgggccctcattcaggcccagccctgctcctgccacgccctggctctggcctggactttctcctatctcctgttcttccgagccctcagcctcctgggcctgcccactcccacgcccttcaccaatgccgtccagctgctgctgacgctgaagctggtgagcctggccagtgaagtccaggacctgcatctggcccagaggaaggaaatggcctcaggcttcagcaaggggcccaccctggggctgctgcccgacgtgccctccctgatggagacactcagctacagctactgctacgtgggaatcatgacaggcccgttcttccgctaccgcacctacctggactggctggagcagcccttccccggggcagtgcccagcctgcggcccctgctgcgccgcgcctggccggccccgctcttcggcctgctgttcctgctctcctctcacctcttcccgctggaggccgtgcgcgaggacgccttctacgcccgcccgctgcccgcccgcctcttctacatgatccccgtcttcttcgccttccgcatgcgcttctacgtggcctggattgccgccgagtgcggctgcattgccgccggctttggggcctaccccgtggccgccaaagcccgggccggaggcggccccaccctccaatgcccaccccccagcagtccggagaaggcggcttccttggagtatgactatgagaccatccgcaacatcgactgctacagcacagatttctgcgtgcgggtgcgcgatggcatgcggtactggaacatgacggtgcagtggtggctggcgcagtatatctacaagagcgcacctgcccgttcctatgtcctgcggagcgcctggaccatgctgctgagcgcctactggcacggcctccacccgggctactacctgagcttcctgaccatcccgctgtgcctggctgccgagggccggctggagtcagccctgcgggggcggctgagcccagggggccagaaggcctgggactgggtgcactggttcctgaagatgcgcgcctatgactacatgtgcatgggcttcgtgctgctctccttggccgacacccttcggtactgggcctccatctacttctgtatccacttcctggccctggcagccctggggctggggctggctttaggtgggggcagccccagccggcggaaggcagcatcccagcccaccagccttgccccggagaagctccgggaggagtaagctgtcacgacgctccctctgccagctggtcccgggaattctgtgaaccaggctgctgtctcctccccagaaagagtccttaccttggagagggtcctggagagaatttcctcttccccagctaaataccctgcctgcaactgaagcagacccgggggtgtcctccctgccctctgcccagaggccacctccactcctacaaaatcaaagtattgtccagacaagagtcactggcccctgctccagcttctgggtatccagagagcactgcacttccccaaaacggaaggggcccctgggcagtgggttttgggcaaattccctttctttgcatccacaatgtggggtcggagcttgggggcaggtcctgggagtgggaagcctcttccttgtgtctttcgctccacttttagctcatcgcaccaatattgcagacttggaaggaagcataagcttcccatttcacaaaggggaaactgaggtgcgggtgcgcgggcctggggacggccgtcccatggcttccatctgagccacctcgggaccccagcactcctggcgccctcttctcatcgcttggcctatgacaggtcaccgtgtgtaaatctttcccaataaagtgttgcacaaag

SEQ ID NO: 3 - protein, 473 aa, Mus sp.
MTPEEWTYLMVLLISIPVGFLFKKAGPGLKRWGAAAVGLGLTLFTCGPHSLHSLITILGTWALIQAQPCSCHALALAWTFSYLLFFRALSLLGLPTPTPFTNAVQLLLTLKLVSLASEVQDLHLAQRKEIASGFHKEPTLGLLPEVPSLMETLSYSYCYVGIMTGPFFRYRTYLDWLEQPFPEAVPSLRPLLRRAWPAPLFGLLFLLSSHLFPLEAVREDAFYARPLPTRLFYMIPVFFAFRMRFYVAWIAAECGCIAAGFGAYPVAAKARAGGGPTLQCPPPSSPEIAASLEYDYETIRNIDCYGTDFCVRVRDGMRYWNMTVQWWLAQYIYKSAPFRSYVLRSAWTMLLSAYWHGLHPGYYLSFMTIPLCLAAEGYLESALRRHLSPGGQKAWDWVHWFLKMRAYDYMCMGFVLLSMADTLRYWASIYFWVHFLALACLGLGLVLGGGSPSKRKTPSQATSSQAKEKLREE

SEQ ID NO: 4 - protein, 472 aa, Homo sapiens
MSPEEWTYLVVLLISIPIGFLFKKAGPGLKRWGAAAVGLGLTLFTCGPHTLHSLVTILGTWALIQAQPCSCHALALAWTFSYLLFFRALSLLGLPTPTPFTNAVQLLLTLKLVSLASEVQDLHLAQRKEMASGFSKGPTLGLLPDVPSLMETLSYSYCYVGIMTGPFFRYRTYLDWLEQPFPGAVPSLRPLLRRAWPAPLFGLLFLLSSHLFPLEAVREDAFYARPLPARLFYMIPVFFAFRMRFYVAWIAAECGCIAAGFGAYPVAAKARAGGGPTLQCPPPSSPEKAASLEYDYETIRNIDCYSTDFCVRVRDGMRYWNMTVQWWLAQYIYKSAPARSYVLRSAWTMLLSAYWHGLHPGYYLSFLTIPLCLAAEGRLESALRGRLSPGGQKAWDWVHWFLKMRAYDYMCMGFVLLSLADTLRYWASIYFCIHFLALAALGLGLALGGGSPSRRKAASQPTSLAPEKLREE

SEQ ID NO: 5 - DNA, 2275 bp, Homo sapiens
  source 1..2275: mol_type = genomic DNA
gggacctcctagaaagatcgcgacaggcatcttgcggcagactggtgttctgggtacctttcttgcggtgctgtaactcgtacagccgcggctctcggggcctggaccgcgcagccctgccggcgccgtccagaacgggcagtgcgggggcgtgctgagctggggaggcgtggcgcgagccgaggcggcctcgaaaaggagctccgcagttttctggcccacggacggttcagaccatgacacccgaagaatggacatatctaatggtccttcttatctccatccctgttggcttcctctttaagaaagctggacctgggctgaagagatggggggcagcagctgtgggcctggggctcaccttattcacctgtggcccccacagtttgcattctctgatcaccatcttgggaacctgggccctcattcaggcccagccctgctcctgccatgccctggctcttgcctggaccttctcctatctcctcttcttccgagccctcagcctgctgggcctgcccactcccacgcccttcaccaatgctgtccagctgctgttgacactgaagttggtgagtctagctagtgaagtccaggatctgcatctggctcagagaaaggaaatagcctccggcttccacaaggagcctacgctgggcctcctccctgaggtcccctctttgatggagacactcagctatagctactgttacgtgggaatcatgacaggcccattcttccgctaccgcacctacctggattggctggaacagcccttcccggaagccgtgcccagcctgaggcccctgctgcgccgcgcctggccagccccgctctttggcctgctcttcctgctgtcctcccatctcttcccactggaagctgtgcgtgaggacgccttctacgcccgcccgctgcccacccgcctcttctacatgatcccggtcttcttcgccttccgcatgcgcttctacgttgcctggattgcggccgagtgcggttgcattgccgcgggcttcggggcctaccctgtggctgccaaagcccgggccgggggcggccccaccctccaatgcccaccccctagcagtccggagattgcagcttccctggagtatgactatgagaccatccgtaacatcgactgctatggcacagacttctgcgtgcgtgtgcgggatggcatgcgatactggaacatgaccgtgcagtggtggctggcacagtacatctacaagagcgcacctttctgctcctacgttttgaggagtgcctggaccatgctgttgagtgcctactggcatggcctccaccctggttactacctaagcttcatgaccatcccgctgtgcctggctgctgagggctatttggagtcagccttgcggagacacctgagccccgggggccagaaagcctgggactgggtccactggttcctgaagatgcgtgcctacgactacatgtgcatgggctttgtgctcctttccatggctgacacactccggtactgggcctccatctacttctgggtccactttctagccctggcctgcttggggctggggctggttttgggtgggggcagccccagcaagaggaagacaccatcccaggctaccagcagccaagcgaaggaaaagctccgggaagagtgagctctgctgcattggcctgccttccagttcaagcttttttgggaattccatgaaccaggctgtttgtttggggtttttgtttgtttgtttgtttgtttcctttacccagcaagaatcccttgtttggctaagagcctggagaggattccctcttcccaaataattcctctgccttctatttaaagctaaggtatccttctcttgggctctctcagcatcttgaccttttcagaccttcctctgctaacatcagggtattactatccactcttgaacctattatctctgcaacaatcttcagatgttcaaaaagccacacttcccaaaaatgcccttgcagggaccagtggtcatctggcatcttagacagactcccagtgggtccccagtatgggggcaggaacttcagggccaggttctgggagaggggagggatagccttcttgttttcctctttgtttttatcatcacaccagtgtttcagagaccatggtcttacacatgctgaaggagaagctaaaatgtgagaagccccaggggagcttgctcttacagcagcttctgcctgagccatttctgggctccccatacaacctaccacccagtgtcatccttgg

SEQ ID NO: 6 - DNA, 25 bp, synthetic construct
  source 1..25: mol_type = other DNA
  modified_base 1: mod_base = OTHER; note = 2'-O-methyl nucleotide
  modified_base 2: mod_base = OTHER; note = 2'-O-methyl nucleotide
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 20: mod_base = OTHER; note = 2'-O-methyl nucleotide
  modified_base 21: mod_base = OTHER; note = 2'-O-methyl nucleotide
  modified_base 22: mod_base = OTHER; note = 2'-O-methyl nucleotide
  modified_base 23: mod_base = OTHER; note = 2'-O-methyl nucleotide
  modified_base 24: mod_base = OTHER; note = 2'-O-methyl nucleotide
  modified_base 25: mod_base = OTHER; note = 2'-O-methyl nucleotide
  misc_feature 1..5: note = RNA
  misc_feature 6..19: note = DNA
  misc_feature 20..25: note = RNA
ggcgtttctctgatatcatcttcca

SEQ ID NO: 7 - DNA, 25 bp, synthetic construct
  source 1..25: mol_type = other DNA
  modified_base 1: mod_base = OTHER; note = 2'-O-methyl nucleotide
  modified_base 2: mod_base = OTHER; note = 2'-O-methyl nucleotide
  modified_base 3: mod_base = OTHER; note = 2'-O-methyl nucleotide
  modified_base 4: mod_base = OTHER; note = 2'-O-methyl nucleotide
  modified_base 5: mod_base = OTHER; note = 2'-O-methyl nucleotide
  modified_base 20: mod_base = OTHER; note = 2'-O-methyl nucleotide
  modified_base 21: mod_base = OTHER; note = 2'-O-methyl nucleotide
  modified_base 22: mod_base = OTHER; note = 2'-O-methyl nucleotide
  modified_base 23: mod_base = OTHER; note = 2'-O-methyl nucleotide
  modified_base 24: mod_base = OTHER; note = 2'-O-methyl nucleotide
  modified_base 25: mod_base = OTHER; note = 2'-O-methyl nucleotide
  misc_feature 1..5: note = RNA
  misc_feature 6..19: note = DNA
  misc_feature 20..25: note = RNA
caagtcactggacttgaataagaat

SEQ ID NO: 8 - DNA, 25 bp, Mus sp.
  source 1..25: mol_type = genomic DNA
  misc_feature 1..23: note = RNA
  misc_feature 24..25: note = DNA
tgccttctatttaaagctaaggtat

SEQ ID NO: 9 - RNA, 25 nt, Homo sapiens
  source 1..25: mol_type = genomic RNA
ctactgctacgtgggaatcatgaca

SEQ ID NO: 10 - DNA, 54 bp, synthetic construct
  source 1..54: mol_type = other DNA
caccatgatggagacactcagctatatcaagagtatagctgagtgtctccatca

SEQ ID NO: 11 - RNA, 36 nt, synthetic construct
  source 1..36: mol_type = other RNA
  modified_base 1: mod_base = OTHER; note = AltR1 proprietary end-modification - proprietary/trademark name of IDT
  modified_base 36: mod_base = OTHER; note = AltR2 proprietary end-modification - proprietary/trademark name of IDT
acaaccacgtagggctgcgtgttttagagctatgct

SEQ ID NO: 12 - RNA, 36 nt, synthetic construct
  source 1..36: mol_type = other RNA
  modified_base 1: mod_base = OTHER; note = AltR1 proprietary end-modification - proprietary/trademark name of IDT
  modified_base 36: mod_base = OTHER; note = AltR2 proprietary end-modification - proprietary/trademark name of IDT
tcgatatgctacacgagaaggttttagagctatgct

SEQ ID NO: 13 - DNA, 25 bp, synthetic construct
  source 1..25: mol_type = other DNA
  misc_feature 1..23: note = RNA
  misc_feature 24..25: note = DNA
tgccttctatttaaagctaaggtat

SEQ ID NO: 14 - RNA, 27 nt, synthetic construct
  source 1..27: mol_type = other RNA
tgtcatgattcccacgtagcagtagct

SEQ ID NO: 15 - DNA, 25 bp, synthetic construct
  source 1..25: mol_type = other DNA
  misc_feature 1..23: note = RNA
  misc_feature 24..25: note = DNA
ggcgtttctctgatatcatcttcca

SEQ ID NO: 16 - DNA, 25 bp, synthetic construct
  source 1..25: mol_type = other DNA
  misc_feature 1..23: note = RNA
  misc_feature 24..25: note = DNA
caagtcactggacttgaataagaat

SEQ ID NO: 17 - DNA, 25 bp, synthetic construct
  source 1..25: mol_type = other DNA
  misc_feature 1..23: note = RNA
  misc_feature 24..25: note = DNA
gtgtttccatgaaataatcactatt

SEQ ID NO: 18 - DNA, 21 bp, synthetic construct
  source 1..21: mol_type = other DNA
tcagtcaacgggggacataaa

SEQ ID NO: 19 - DNA, 21 bp, synthetic construct
  source 1..21: mol_type = other DNA
ggggctgtactgcttaaccag

SEQ ID NO: 20 - DNA, 23 bp, synthetic construct
  source 1..23: mol_type = other DNA
atccgtaacatcgactgctatgg

SEQ ID NO: 21 - DNA, 20 bp, synthetic construct
  source 1..20: mol_type = other DNA
cggaaaggtgcgctcttgta

SEQ ID NO: 22 - DNA, 24 bp, synthetic construct
  source 1..24: mol_type = other DNA
atgctcccagggctgttttcccat

SEQ ID NO: 23 - DNA, 25 bp, synthetic construct
  source 1..25: mol_type = other DNA
gtggtgccagatcttttccatgtcg

SEQ ID NO: 24 - DNA, 19 bp, synthetic construct
  source 1..19: mol_type = other DNA
gctcctcttaggggccact

SEQ ID NO: 25 - DNA, 19 bp, synthetic construct
  source 1..19: mol_type = other DNA
ccacgtctcaccattgggg

SEQ ID NO: 26 - DNA, 21 bp, synthetic construct
  source 1..21: mol_type = other DNA
gtaacttcgtgcctagcaaca

SEQ ID NO: 27 - DNA, 23 bp, synthetic construct
  source 1..23: mol_type = other DNA
cctttgtcagaatactgagcagc

SEQ ID NO: 28 - DNA, 23 bp, synthetic construct
  source 1..23: mol_type = other DNA
ctgtaacatggaaactggggaaa

SEQ ID NO: 29 - DNA, 23 bp, synthetic construct
  source 1..23: mol_type = other DNA
ccatagctgaactgaaaaccacc

SEQ ID NO: 30 - DNA, 20 bp, synthetic construct
  source 1..20: mol_type = other DNA
acctagacctcgccaaaggt

SEQ ID NO: 31 - DNA, 20 bp, synthetic construct
  source 1..20: mol_type = other DNA
gcacggataagcaggtttgt

SEQ ID NO: 32 - DNA, 21 bp, synthetic construct
  source 1..21: mol_type = other DNA
cttgtggaatgccttgtgatt

SEQ ID NO: 33 - DNA, 19 bp, synthetic construct
  source 1..19: mol_type = other DNA
agccgaagcagcacatgat

SEQ ID NO: 34 - DNA, 18 bp, synthetic construct
  source 1..18: mol_type = other DNA
gccgtgaactgggtcgaa

SEQ ID NO: 35 - DNA, 21 bp, synthetic construct
  source 1..21: mol_type = other DNA
gcatatatagcaatgtctcct

SEQ ID NO: 36 - DNA, 19 bp, synthetic construct
  source 1..19: mol_type = other DNA
aggctgtgggctccatagg

SEQ ID NO: 37 - DNA, 17 bp, synthetic construct
  source 1..17: mol_type = other DNA
tgcggtccagggtcatc

SEQ ID NO: 38 - DNA, 20 bp, synthetic construct
  source 1..20: mol_type = other DNA
gcctctcagaatggtcgtct

SEQ ID NO: 39 - DNA, 20 bp, synthetic construct
  source 1..20: mol_type = other DNA
cgcatgtcccagaataagga

SEQ ID NO: 40 - DNA, 20 bp, synthetic construct
  source 1..20: mol_type = other DNA
cacgccagtgccaaattaga

SEQ ID NO: 41 - DNA, 15 bp, synthetic construct
  source 1..15: mol_type = other DNA
cccaaggccgcttcg

SEQ ID NO: 42 - DNA, 20 bp, synthetic construct
  source 1..20: mol_type = other DNA
tcttctgctgcgtgacaacc

SEQ ID NO: 43 - DNA, 21 bp, synthetic construct
  source 1..21: mol_type = other DNA
gagaaaccagcttggaaccag

SEQ ID NO: 44 - DNA, 19 bp, synthetic construct
  source 1..19: mol_type = other DNA
tttctctgcctctgccaac

SEQ ID NO: 45 - DNA, 22 bp, synthetic construct
  source 1..22: mol_type = other DNA
tctcattgatcacctgtccatc

SEQ ID NO: 46 - DNA, 22 bp, synthetic construct
  source 1..22: mol_type = other DNA
attaacccaactatgaagtgcc

SEQ ID NO: 47 - DNA, 21 bp, synthetic construct
  source 1..21: mol_type = other DNA
ctgtctcctcactgaaggctc

SEQ ID NO: 48 - DNA, 21 bp, synthetic construct
  source 1..21: mol_type = other DNA
ttgatgccattaccagtctcc

SEQ ID NO: 49 - DNA, 20 bp, synthetic construct
  source 1..20: mol_type = other DNA
acatggttgggaagttctgg

SEQ ID NO: 50 - DNA, 20 bp, synthetic construct
  source 1..20: mol_type = other DNA
tcagagccaaagcagtgagc

SEQ ID NO: 51 - DNA, 23 bp, synthetic construct
  source 1..23: mol_type = other DNA
gccgtgtagataaactcgatgtc

SEQ ID NO: 52 - DNA, 20 bp, synthetic construct
  source 1..20: mol_type = other DNA
cctgcttcattcccctcttc

SEQ ID NO: 53 - DNA, 21 bp, synthetic construct
  source 1..21: mol_type = other DNA
tgtcatgctcaccgtagaaag

SEQ ID NO: 54 - DNA, 21 bp, synthetic construct
  source 1..21: mol_type = other DNA
gatcctggaaaaaggacctgg

SEQ ID NO: 55 - DNA, 22 bp, synthetic construct
  source 1..22: mol_type = other DNA
gtctgaagagggtgtcaaatcc

SEQ ID NO: 56 - DNA, 23 bp, synthetic construct
  source 1..23: mol_type = other DNA
ttaaaaacctggatcggaaccaa

SEQ ID NO: 57 - DNA, 23 bp, synthetic construct
  source 1..23: mol_type = other DNA
gcattagcttcagatttacgggt

SEQ ID NO: 58 - DNA, 20 bp, synthetic construct
  source 1..20: mol_type = other DNA
tccgcaacatcgactgctac

SEQ ID NO: 59 - DNA, 19 bp, synthetic construct
  source 1..19: mol_type = other DNA
cgcaggacataggaacggg

SEQ ID NO: 60 - DNA, 20 bp, synthetic construct
  source 1..20: mol_type = other DNA
aactcgctggctatctcggt

SEQ ID NO: 61 - DNA, 20 bp, synthetic construct
  source 1..20: mol_type = other DNA
gccctcataatgcagggact

SEQ ID NO: 62 - DNA, 21 bp, synthetic construct
  source 1..21: mol_type = other DNA
agagtgtctgcggatacttcc

SEQ ID NO: 63 - DNA, 21 bp, synthetic construct
  source 1..21: mol_type = other DNA
ccaacagtgtaggtcttggtg

SEQ ID NO: 64 - DNA, 21 bp, synthetic construct
  source 1..21: mol_type = other DNA
gagggccaagacgaagacatc

SEQ ID NO: 65 - DNA, 21 bp, synthetic construct
  source 1..21: mol_type = other DNA
cagatcacgtcatcgcacaac

SEQ ID NO: 66 - DNA, 21 bp, synthetic construct
  source 1..21: mol_type = other DNA
caacggatttggtcgtattgg

SEQ ID NO: 67 - DNA, 19 bp, synthetic construct
  source 1..19: mol_type = other DNA
tgacggtgccatggaattt

SEQ ID NO: 68 - DNA, 21 bp, synthetic construct
  source 1..21: mol_type = other DNA
tgtgccgctagaggtgaaatt

SEQ ID NO: 69 - DNA, 19 bp, synthetic construct
  source 1..19: mol_type = other DNA
tggcaaatgctttcgcttt

SEQ ID NO: 70 - protein, 6 aa, synthetic construct
HHHHHH